(19) United States
(12) Patent Application Publication
(10) Pub. No.: US 2010/0029636 A1
(43) Pub. Date: Feb. 4, 2010

(54) Lck inhibitors

(76) Inventors: Peter Buehlmayer, Arlesheim (CH); Werner Breitenstein, Basel (CH); Pascal Furet, Thann (FR); Bernard Pirard, Hegenheim (DE); Anette von Matt, Biel-Benken (CH); Thomas Zoller, Andolsheim (FR)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/443,229

(22) PCT Filed: Sep. 26, 2007
(86) PCT No.: PCT/EP07/08390

§ 371 Date: Mar. 27, 2009

(30) Foreign Application Priority Data
Sep. 28, 2006 (EP) 06121416.9

(51) Int. Cl. A61K 31/5377; C07D 487/04; A61K 31/4985; C07D 413/14; A61P 29/00

(52) U.S. Cl. 514/233.2; 544/281; 514/252.16; 544/117

(57) ABSTRACT

The invention relates to novel pyrazolo[1,5-A]pyrimidine compounds of Formula I

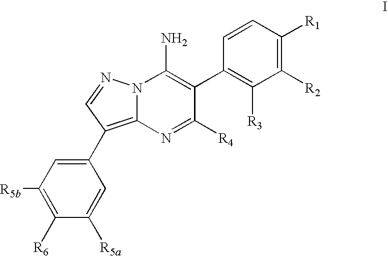

in which all the variables are as defined in the specification; to their preparation and to their use as Lck kinase inhibitors.

The present invention relates to pyrazolo-pyrimidine derivatives, process for their production, their uses and pharmaceutical compositions containing them.

More particularly, the invention provides a compound of formula I

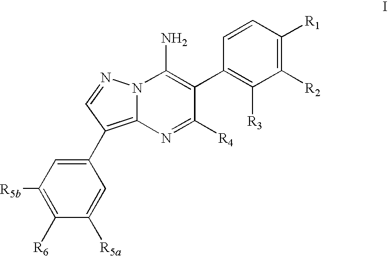

wherein
each of R₁ and R₂, independently, is H; OH; NH₂; NO₂; C₁₋₄alkyl; C₁₋₄alkoxy; aryl-C₁₋₄alkoxy; NR₁₁SO₂R₁₂; NR₁₃COR₁₄; NR₁₅COOR₁₆; or NR₁₇CONR₁₈R₁₉; provided that at least one of R₁ and R₂ is other than H;
R₃ is H; halogen; C₁₋₄alkyl; or C₁₋₄alkoxy;
R₄ is H; optionally substituted C₁₋₄alkyl; or C₁₋₄alkoxy optionally substituted by NH₂, NH(C₁₋₄alkyl) or N(C₁₋₄alkyl)₂;
each of R₅ₐ, R_{5b} and R₆, independently, is H; OH; OR_{c} wherein R_{c} is C₁₋₄alkyl; or a residue of formula (a)

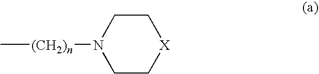

provided that at least one of R₅ₐ, R_{5b} and R₆ is other than H;
R₁₁ is H; or optionally substituted C₁₋₄alkyl;
R₁₂ is C₁₋₈alkyl; C₃₋₈cycloalkyl; optionally substituted aryl or aryl-C₁₋₄alkyl; heterocyclyl; optionally substituted heteroaryl or heteroaryl-C₁₋₄alkyl;
R₁₃ is H; or optionally substituted C₁₋₄alkyl;
R₁₄ is optionally substituted C₁₋₈alkyl; optionally substituted C₃₋₈cycloalkyl; optionally substituted aryl or aryl-C₁₋₄alkyl; or optionally substituted heteroaryl or heteroaryl-C₁₋₄alkyl;
R₁₅ is H; or C₁₋₄alkyl;
R₁₆ is optionally substituted C₁₋₈alkyl; C₃₋₆alkenyl; C₃₋₆alkynyl; optionally substituted C₃₋₈cycloalkyl; optionally substituted aryl or aryl-C₁₋₄alkyl; or optionally substituted heteroaryl-C₁₋₄ alkyl;
each of R₁₇ and R₁₈, independently, is H; or C₁₋₄alkyl;
R₁₉ is C₁₋₈alkyl optionally substituted by halogen or cyano; C₃₋₈cycloalkyl; aryl or aryl-C₁₋₄, each optionally ring-substituted by halogen, halo-C₁₋₄alkyl, halo-C₁₋₄alkoxy and/or heterocyclyl; or optionally substituted heteroaryl or heterocyclyl; or R₁₈ and R₁₉ form together with the nitrogen atom to which they are bound an optionally substituted heterocyclyl residue;
n is 0 or 1;
X is CR₂₀R₂₁ wherein each of R₂₀ and R₂₁, independently, is H or C₁₋₄alkyl; 0; or N—R₂₂ wherein R₂₂ is H; optionally substituted C₁₋₄alkyl; optionally substituted aryl-C₁₋₄alkyl; optionally substituted heteroaryl-C₁₋₄alkyl; optionally substituted heterocyclyl; SO₂—C₁₋₄alkyl; CO—R₂₃— wherein R₂₃ is C₁₋₄alkyl optionally substituted by halogen, heterocyclyl, heteroaryl, amino and/or COOH, or R₂₃ is optionally substituted aryl, heteroaryl or heterocyclyl; or CO—CHR₂₄—NR₂₅R₂₆ wherein R₂₄ is H, C₁₋₈alkyl optionally substituted by OH, NH₂, NH(C₁₋₄alkyl), N(C₁₋₄alkyl)₂, COOH, carbamoyl, CONH(C₁₋₄alkyl), CON(C₁₋₄alkyl)₂ or optionally substituted aryl or heteroaryl, R₂₅ is H or C₁₋₄alkyl, and R₂₆ is H, C₁₋₄alkyl, C₁₋₄alkoxy-carbonyl or aryl-C₁₋₄alkoxycarbonyl wherein aryl may be optionally substituted,
   provided that
   i. when either R₅ₐ, R_{5b} or R₆ is a residue of formula (a) wherein X is NCH₃ and n is 0, then either R₂ is other than NH—SO₂—CH₃ or NH—SO₂-4-fluoro-phenyl or R₁ is other than NH—SO₂-2,3-dichloro-phenyl or R₃ or R₄ is other than H;
   ii. when either R₅ₐ, R_{5b} or R₆ is a residue of formula (a) wherein X is NCH₃ and n is 0, then either R₂ is other than NH—CO—CH₃ or R₃ or R₄ is other than H;
   iii. when either R₅ₐ, R_{5b} or R₆ is a residue of formula (a) wherein X is NH or NCH₃ and n is 0, then either R₂ is other than NH—COOC₁₋₂alkyl or R₃ or R₄ is other than H;
   iv. when either R₅ₐ, R_{5b} or R₆ is a residue of formula (a) wherein X is NH or NCH₃ and n is 0, then either R₁ is other than NH—CO—NH-(3-CF₃-4-morpholino-phenyl) or R₂ is other than NH—CO—NH-(3-CF₃-phenyl) or R₃ or R₄ is other than H;
   v. when one of R₁ and R₂ is OH, the other is H, R₄ is H and only one of R₅ₐ, R_{5b} or R₆ is a residue of formula (a) and the remaining being each H, then the residue of formula (a) is other than 4-methyl-piperazinyl;
   vi. when one of R₁ and R₂ is OH, the other is H and only one of R₅ₐ, R_{5b} or R₆ is a 4-methyl-piperazinyl, the remaining being each H, then R₄ is optionally substituted C₁₋₄alkyl; and
   vii. when either R₅ₐ, R_{5b} or R₆ is a residue of formula (a) wherein X is NH or NCH₃ and n is 0, and R₁ is H, then R₂ is other than NH₂ or R₃ or R₄ is other than H; or a salt thereof.

Any alkyl may be straight or branched. Aryl may be phenyl or naphthyl, preferably phenyl. Aryl-C₁₋₄alkyl may be e.g. benzyl or phenethyl, preferably benzyl. Aryl-C₁₋₄alkoxy may be e.g. benzyloxy.

Halogen may be F, Cl or Br. Halo-C₁₋₄alkyl or halo-C₁₋₄alkoxy may be C₁₋₄alkyl or C₁₋₄alkoxy substituted by one or more halogen, e.g. CF₃ or OCF₃.

Heteroaryl may be a mono- or bicyclic aromatic system comprising 1 to 3 heteroatoms selected from N, O and S, e.g. furyl, thienyl, pyrrolyl, imidazolyl, pyrazolyl, thiazolyl, oxazolyl, isoxazolyl, oxadiazolyl, triazolyl, pyridyl, pyridazinyl, pyrimidinyl, pyrazinyl, indolyl, benzothienyl, benzofuryl, benzimidazolyl, benzothiazolyl or indazolyl.

Heterocyclyl is a 5, 6 or 7 membered non-aromatic heterocyclic ring which may be linked via C or N. Examples are e.g. pyrrolidinyl, morpholinyl, piperazinyl or piperidyl. Heterocyclyl may be substituted by e.g. C₁₋₄alkyl on a ring C and/or N atom,

When R₄ is substituted C₁₋₄alkyl, it may be C₁₋₄alkyl substituted by halogen, cyano, C₁₋₄alkoxy, amino, C₁₋₄alkylamino or di-(C₁₋₄alkyl)-amino, and optionally interrupted by —NH—. Preferably the substituent, when present, is attached to a terminal carbon atom.

When R₁₁ or R₁₃ is optionally substituted alkyl, it may be substituted by e.g. NH₂, C₁₋₄alkylamino or di-(C₁₋₄alkyl)amino.

When R₁₂ is substituted aryl, aryl-C₁₋₄alkyl, heteroaryl or heteroaryl-C₁₋₄alkyl, the aryl or heteroaryl ring may be substituted by one or more substituents selected from halogen, CN, C₁₋₄alkyl, halo-C₁₋₄alkyl, C₁₋₄alkoxy, halo-C₁₋₄alkoxy, amino and heteroaryl. Preferably the aryl or heteroaryl, when substituted, have one or two substituents as indicated above.

When R₁₄ is optionally substituted C₁₋₈alkyl or C₃₋₈cycloalkyl, it may be substituted e.g. by halogen, cyano or C₁₋₄alkoxy. Preferably for the alkyl group the substituent is attached to a terminal carbon atom. When R₁₄ is substituted C₃₋₈cycloalkyl, aryl, aryl-C₁₋₄alkyl, heteroaryl or heteroaryl-C₁₋₄alkyl, it may be substituted by one or more substituents selected from e.g. halogen, C₁₋₄alkyl and halo-C₁₋₄alkyl. When R₁₄ is substituted heteroaryl or heteroaryl-C₁₋₄alkyl, the substituent may be attached to a ring C and/or N atom of the heteroaryl; in the latter case, it is preferably C₁₋₄alkyl. Substituted heteroaryl or heteroaryl-C₁₋₄alkyl may be mono- or di-substituted.

When R₁₆ is substituted C₁₋₈alkyl, it may be substituted e.g. by halogen, cyano or C₁₋₄alkoxy. Preferably the substituent is attached to a terminal carbon atom. When R₁₆ is substituted aryl aryl-C₁₋₄ or heteroaryl-C₁₋₄alkyl, it may be substituted by one or more substituents selected e.g. from halogen, halo-C₁₋₄alkyl and C₁₋₄alkyl.

When R₁₉ is substituted heteroaryl, the substituent may be attached to a ring C and/or N atom of the heteroaryl, and may be e.g. halogen, halo-C₁₋₄alkyl or C₁₋₄alkyl.

When R₂₂ is optionally substituted C₁₋₄alkyl, it may be substituted by OH or C₁₋₄alkoxy, preferably on the terminal C. When R₂₂ is optionally substituted heterocyclyl, it may be substituted e.g. by C₁₋₄alkyl, on a C or on the N atom, e.g. piperidinyl optionally N-substituted by CH₃. When R₂₂ is optionally substituted heteroaryl-C₁₋₄alkyl, it may be ring substituted by C₁₋₄alkyl, e.g. methyl.

When R₂₃ is C₁₋₄alkyl substituted by heterocyclyl, it may be substituted on the terminal C atom, e.g. □CH₂-heterocyclyl. When R₂₃ is optionally substituted aryl, it may be substituted e.g. by OH, amino, C₁₋₄alkyl-amino, di-(C₁₋₄alkyl)-amino or amino substituted by aryloxy-carbonyl or arylC₋₄alkoxy-carbonyl. Optionally substituted heteroaryl as R₂₃ may be heteroaryl optionally substituted by C₁₋₄alkyl. Optionally substituted heterocyclyl as R₂₃ may be heterocyclyl with a ring N atom optionally substituted by aryloxy-carbonyl or arylC₋₄alkoxy-carbonyl.

When R₂₄ is substituted C₁₋₄alkyl, it may be e.g. mono-substituted, preferably on the terminal C atom. When R₂₄ is C₁₋₄alkyl substituted by aryl or heteroaryl, such aryl may optionally be substituted by e.g. OH and such heteroaryl may optionally be substituted by e.g. C₁₋₄alkyl. When R₂₆ is aryl-C₋₄alkoxy-carbonyl, aryl may optionally be substituted, e.g. by OH.

Preferred compounds of formula I are those wherein R₁ or R₂, preferably R₁ is NHCOOR₁₆, wherein R₁₆ is C₃₋₈alkyl, e.g. C₄₋₆alkyl, or optionally substituted phenyl or phenyl-C₁₋₄alkyl.

For the compounds of formula I the following significances are preferred independently, collectively or in any combination or sub-combination:

(i) each of R₅ₐ, R_{5b} and R₆, independently, is H; OH; or a residue of formula (a), wherein said residue of formula (a) is as defined hereinabove, provided that at least one of R₅ₐ, R_{5b} and R₆ is other than H;
(ii) each of R₅ₐ, R_{5b} and R₆, independently, is H; or a residue of formula (a), wherein said residue of formula (a) is as defined hereinabove, provided that at least one of R₅ₐ, R_{5b} and R₆ is other than H;
(iii) R₂ is H, OH, C₁₋₄alkyl, or C₁₋₄alkoxy; preferably H, OH or C₁₋₄alkoxy;
(iv) R₁ is NR₁₁SO₂R₁₂; NR₁₃COR₁₄; NR₁₅COOR₁₆; or NR₁₇CONR₁₈R₁₉ wherein the variables R₁₁ to R₁₉ have the meanings provided above;
(v) R₁ is preferably NHCOOR₁₆, wherein R₁₆ is C₃₋₈alkyl, e.g. C₄₋₆alkyl, or optionally substituted phenyl or phenylC₁₋₄alkyl.

The compounds of formula I may exist in free form or in salt form, e.g. addition salts with e.g. organic or inorganic acids, for example trifluoroacetic or hydrochloride acid.

When the compounds of formula I have asymmetric centers in the molecule, e.g. when R₂₂ is CO—CHR₂₄—NR₂₅R₂₆ wherein R₂₄ is other than H, various optical isomers are obtained. The present invention also encompasses enantiomers, racemates, diastereoisomers and mixtures thereof. Moreover, when the compounds of formula I include geometric isomers, the present invention embraces cis-compounds, trans-compounds and mixtures thereof. Similar considerations apply in relation to starting materials exhibiting asymmetric carbon atoms or unsaturated bonds as mentioned above.

The present invention also provides a process for the production of a compound of formula I, comprising

a) reacting a compound of formula II

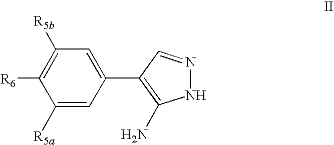

wherein R₅ₐ, R_{5b} and R₆ are as defined above, with a compound of formula III

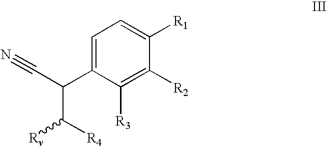

wherein R₁ to R₄ are as defined above and Rᵥ is e.g. OH or substituted amino, e.g. N(CH₃)₂; or
b) converting a compound of formula I into another compound of formula I
and recovering the resulting compound of formula I in free or in form of a salt, and, where required, converting the compound of formula I obtained in free form into the desired salt form, or vice versa.

The process steps a) and b) may be performed according to methods known in the art, or as disclosed below in the Examples.

Examples of conversion of a compound of formula I into another compound of formula I may include e.g.
i) for the production of a compound of formula I wherein R₁ or R₂ is amino reducing a compound of formula I wherein R₁ or R₂ is NO₂, e.g. by hydrogenation.
ii) for the production of a compound of formula I wherein R₁ or R₂ is NR₁₁SO₂R₁₂, NR₁₃COR₁₄, NR₁₅COOR₁₆, or NR₁₇CONR₁₈R₁₉ reacting a compound of formula I wherein R₁ or R₂ is amino, with an appropriate acylating agent. The reaction may be performed in accordance with methods known in the art or e.g. as disclosed in the Examples.
iii) for the production of a compound of formula I comprising a residue of formula (a) wherein R₂₂ is CO—R₂₃ or CO—CHR₂₄—NR₂₅R₂₆, reacting a compound of formula I wherein R₂₂ is H with an appropriate acylating agent. The reaction may be performed in accordance with methods known in the art or e.g. as disclosed in the Examples.

Compounds of formula II, used as starting materials, may be produced e.g. as disclosed in following reaction scheme:

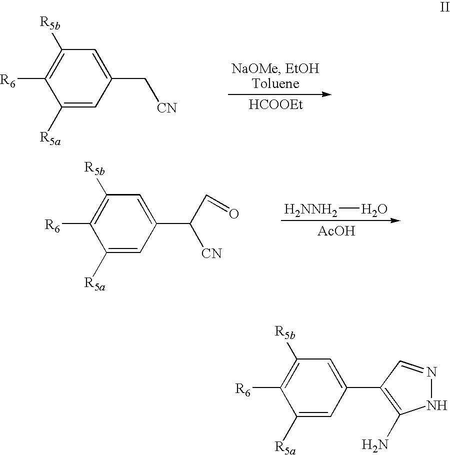

wherein R₅ₐ, R_{5b} and R₆ are as defined above.

Compounds of formula II, used as starting materials, may be produced e.g. as disclosed in following reaction scheme:

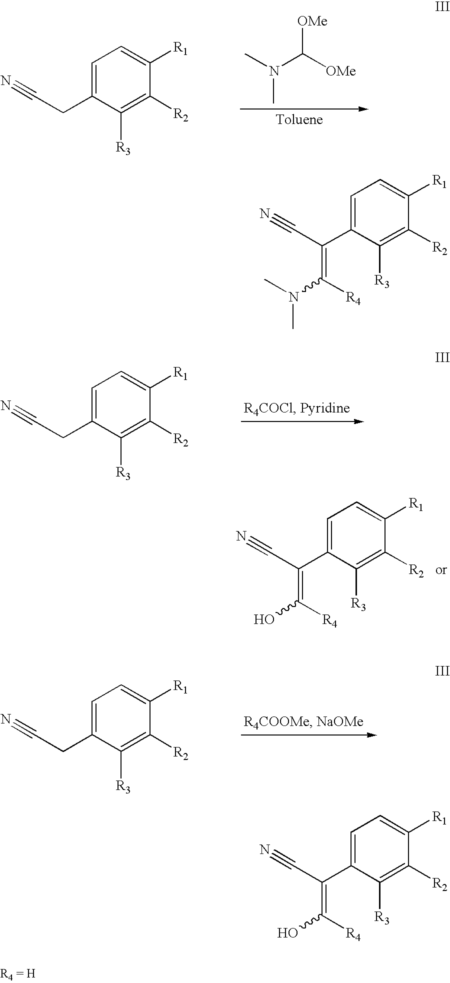

R₁ to R₄ being as defined above.

Insofar as the production of the starting materials is not particularly described, the compounds are known or may be prepared analogously to methods known in the art or as disclosed in the Examples hereinafter.

The following examples illustrate the invention without any limitation.

### EXAMPLE 1

### 3-[4-(4-Methyl-piperazin-1-yl)-phenyl]-6-(4-nitro-phenyl)-pyrazolo[1,5-a]pyrimidin-7-ylamine

### A) [4-(4-Methyl-piperazin-1-yl)-phenyl]-acetonitrile

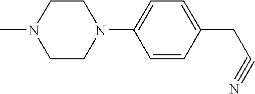

Under argon atmosphere 4-bromophenyl acetonitrile (9.04 g, 46.1 mM), N-methyl-piperazine (5.55 g, 55.4 mM), and (2-biphenyl)di-t-butylphosphin (2.08 g, 6.97 mM) are dissolved in 1,2-dimethoxyethane (77 ml). Palladium (II) acetate (543 mg, 2.42 mM) and potassium phosphate (13.9 g, 65.6 mM) are added and the reaction mixture stirred at 90° C. for 23 h. After cooling down to room temperature, water and ethyl acetate are added, the layers are separated and the aqueous layer is extracted several times with ethyl acetate. The combined organic phases are washed with brine, and dried over Na₂SO₄. The solvent is removed in vacuo and the residue is purified by chromatography (ethylacetate/ethanol/ammonia=95:9.5:0.5) to give the desired product as brown powder, M⁺H⁺=216.

### B) 3-Hydroxy-2-[4-(4-methyl-piperazin-1-yl)-phenyl]-acrylonitrile

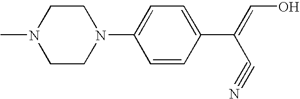

Sodium (597 mg, 26.0 mM) is dissolved in ethanol (34 ml), [4-(4-methyl-piperazin-1-yl)-phenyl]-acetonitrile (3.73 g, 17.3 mM) and ethyl formate (1.92 g, 26.0 mM) are added and the reaction mixture stirred at 75° C. for 1.5 h. After cooling to room temperature, diethyl ether is added and the product is isolated by filtration as brown powder, M⁺H⁺=244.

### C) 4-[4-(4-Methyl-piperazin-1-yl)-phenyl]-2H-pyrazol-3-ylamine

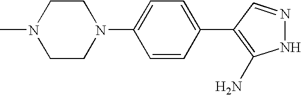

To a solution of [4-(4-methyl-piperazin-1-yl)-phenyl]-acetonitrile (3.65 g, 13.8 mM) in acetic acid (53 ml) hydrazine monohydrate (1.72 g, 34.4 mM) is added. The reaction mixture is stirred at 125° C. for 1.5 h, cooled to room temperature, water (103 ml) and fuming HCl (10.7 ml) is added and the mixture stirred at 110° C. for 1 h. The reaction mixture is cooled to 0° C., conc. ammonia (80 ml) is added and the product extracted several times with CH₂Cl₂/MeOH=9:1. The combined organic layers are dried over Na₂SO₄ and the solvent is removed in vacuo to give the product as brown powder, M⁺H⁺=258.

### D) 3-Dimethylamino-2-(4-nitro-phenyl)-acrylonitrile

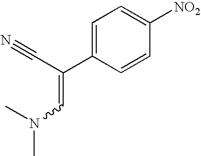

Dimethylformamide dimethylacetale (6.67 g, 30.8 mM) is added to a solution of 4-nitrophenyl acetonitrile (2.50 g, 15.4 mM) in toluene (50 ml) and stirred at 120° C. for 1.5 h. After cooling to room temperature hexane is added, the reaction mixture stirred for 10 min. The precipitate is collected by filtration, washed with hexane and dried in vacuo to give the product as green crystals, M⁺H⁺=218.

### E) 3-[4-(4-Methyl-piperazin-1-yl)-phenyl]-6-(4-nitro-phenyl)-pyrazolo[1,5-a]pyrimi-din-7-ylamine

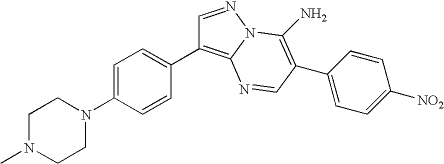

4-[4-(4-Methyl-piperazin-1-yl)-phenyl]-2H-pyrazol-3-ylamine (1.50 g, 5.83 mM) and 3-dimethylamino-2-(4-nitro-phenyl)-acrylonitrile (1.27 g, 5.83 mM) in acetic acid (11.3 ml) and 1.25 M HCl in ethanol (11.3 ml) are stirred at 120° C. for 26 h. After cooling to room temperature, methanol (40 ml) is added and the reaction mixture stirred for 20 min. The precipitate is collected by filtration, washed with methanol and dried in vacuo to yield the product as red crystals, M⁺H⁺=430.

By following the above procedure but using the appropriate starting materials, the following compounds may be prepared:

### EXAMPLE 2

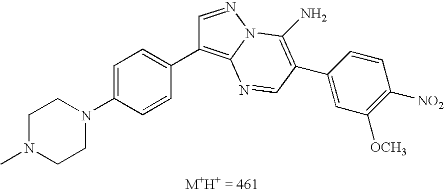

### EXAMPLE 3

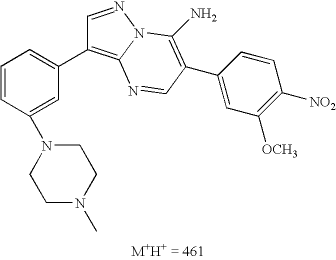

### EXAMPLE 4

### 6-(4-Amino-phenyl)-3-[4-(4-methyl-piperazin-1-yl)-phenyl]-pyrazolo[1,5-a]pyrimi-din-7-ylamine)

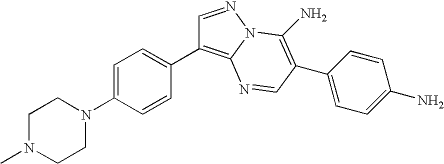

The compound of Example 1 (1.00 g, 2.33 mM) is dissolved in methanol/THF=3:2 (750 ml), palladium on carbon 10% (0.28 g, 10%) is added and the reaction mixture hydrogenated at room temperature for 18 h. The reaction mixture is filtrated over celite and the solvent is removed from the filtrate in vacuo. Diethylether is added to the residue, and the product is isolated by filtration, washed with ether and dried to afford the desired product as brown crystals, M⁺H⁺=400.

By following the procedure of above Examples but using the appropriate starting materials, the following compounds may be prepared:

### EXAMPLE 5

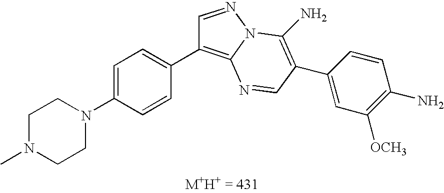

### EXAMPLE 6

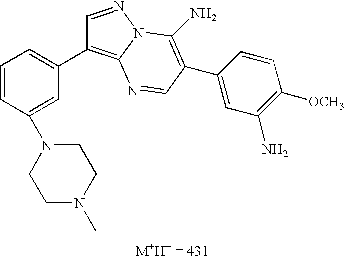

### EXAMPLE 7

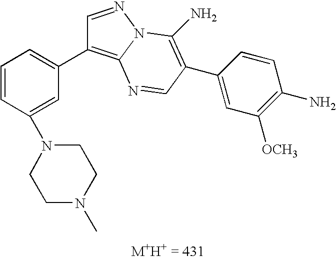

### EXAMPLE 8

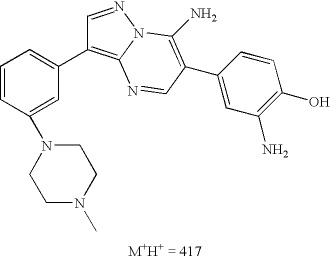

### EXAMPLE 9

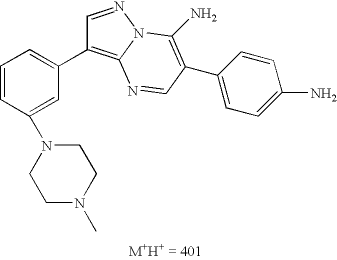

### EXAMPLE 10

### (4-{7-Amino-3-[4-(4-methyl-piperazin-1-yl)-phenyl]-pyrazolo[1,5-a]pyrimidin-6-yl}-phenyl)-carbamic acid isobutyl ester

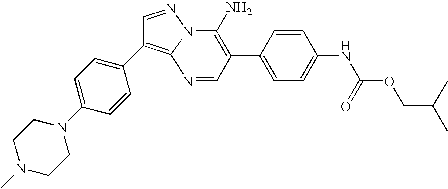

To a solution of the compound of Example 4 (115 mg, 0.29 mM) in pyridine/CH₂Cl₂ 1:1 (2 ml) isobutyl chloroformate (48 mg, 0.35 mM) is added and the reaction mixture stirred at room temperature for 1 h. Isobutyl chloroformate (48 mg, 0.35 mM) is added again, the reaction mixture stirred at 60° C. for 1 h, isobutyl chloroformate (48 mg, 0.35 mM) is added a third time and the reaction mixture stirred at 60° C. for 30 min. After cooling to room temperature, ethyl acetate and sat. NaHCO₃ solution are added and the layers are separated. The aqueous phase is extracted several times with ethyl acetate. The combined organic layers are washed with brine, dried over Na₂SO₄, and the solvent is removed in vacuo. The product is purified by preparative HPLC (H₂O with 0.1% TFA, 100%, 3 min; to H₂O/CH₃CN with 0.1% TFA, 1:9, inert 22 min; H 20/CH₃CN with 0.1% TFA, 1:9, 5 min) to give the desired product as yellow crystals, MH⁺=501.

By following the procedure of above Examples but using the appropriate starting materials, the compounds of formula X₁ may be prepared

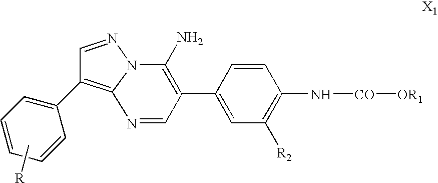

wherein R, R₁ and R₂ have the significances as indicated in Table 1 below.

TABLE 1

| Ex. | R | R₁ | R₂ | M⁺H⁺ |
|---|---|---|---|---|
| 11 | 4-(4-methyl-piperazin-1-yl) | p-tolyl | H | 535 |
| 12 | 4-(4-methyl-piperazin-1-yl) | butyl | H | 501 |
| 13 | 3-(4-methyl-piperazin-1-yl) | pentyl | H | 515 |
| 14 | 4-(4-methyl-piperazin-1-yl) | pentyl | H | 515 |
| 15 | 3-(4-methyl-piperazin-1-yl) | butyl | H | 501 |
| 16 | 4-(4-methyl-piperazin-1-yl) | benzyl | H | 535 |
| 17 | 3-(4-methyl-piperazin-1-yl) | hexyl | H | 529 |
| 18 | 4-(4-methyl-piperazin-1-yl) | 4-fluoro-phenyl | H | 535 |
| 19 | 3-(4-methyl-piperazin-1-yl) | p-tolyl | H | 535 |
| 20 | 4-(4-methyl-piperazin-1-yl) | 2,4-xylyl | H | 549 |
| 21 | 3-(4-methyl-piperazin-1-yl) | 4-fluoro-phenyl | H | 539 |
| 22 | 4-(4-methyl-piperazin-1-yl) | isobutyl | H | 501 |
| 23 | 4-(4-methyl-piperazin-1-yl) | 4-chloro-phenyl | H | 554/556 |
| 24 | 4-(4-methyl-piperazin-1-yl) | 4-propyl-phenyl | H | 563 |
| 25 | 4-(4-methyl-piperazin-1-yl) | phenyl | H | 521 |
| 26 | 3-(4-methyl-piperazin-1-yl) | 2,4-xylyl | H | 549 |
| 27 | 3-(4-methyl-piperazin-1-yl) | phenyl | H | 521 |
| 28 | 4-(4-methyl-piperazin-1-yl) | propyl | H | 487 |
| 29 | 4-(4-methyl-piperazin-1-yl) | butyne-3-yl | H | 497 |
| 30 | 3-(4-methyl-piperazin-1-yl) | benzyl | H | 535 |
| 31 | 4-(4-methyl-piperazin-1-yl) | 2-chloro-benzyl | H | 568/570 |
| 32 | 3-(4-methyl-piperazin-1-yl) | 2-chloro-benzyl | H | 568/570 |
| 33 | 3-(4-methyl-piperazin-1-yl) | isobutyl | H | 501 |
| 34 | 3-(4-methyl-piperazin-1-yl) | propyl | H | 487 |
| 35 | 3-(4-methyl-piperazin-1-yl) | butyne-2-yl | H | 497 |
| 36 | 3-(4-methyl-piperazin-1-yl) | naphthyl | H | 571 |
| 37 | 4-(4-methyl-piperazin-1-yl) | 2-methoxy-ethyl | H | 503 |
| 38 | 4-(4-methyl-piperazin-1-yl) | 2,2-dimethyl- | H | 515 |
| | | propyl | | |
| 39 | 4-(4-methyl-piperazin-1-yl) | isopropyl | H | 487 |
| 40 | 3-(4-methyl-piperazin-1-yl) | isopropyl | H | 487 |
| 41 | 4-(4-methyl-piperazin-1-yl) | naphthyl | H | 571 |
| 42 | 3-(4-methyl-piperazin-1-yl) | butyne-3-yl | H | 497 |
| 43 | 3-(4-methyl-piperazin-1-yl) | ethyl | H | 473 |
| 44 | 3-(4-methyl-piperazin-1-yl) | 2,2-dimethyl- | H | 515 |
| | | propyl | | |
| 45 | 4-(4-methyl-piperazin-1-yl) | ethyl | OCH₃ | 503 |
| 46 | 4-(4-methyl-piperazin-1-yl) | 2-ethyl-hexyl | H | 557 |
| 47 | 4-(4-methyl-piperazin-1-yl) | isobutyl | OCH₃ | 531 |
| 48 | 4-(4-methyl-piperazin-1-yl) | methyl | H | 459 |
| 49 | 3-(4-methyl-piperazin-1-yl) | 2-ethyl-hexyl | H | 557 |
| 50 | 3-(4-methyl-piperazin-1-yl) | butyl | H | 503 |
| 51 | 3-(4-methyl-piperazin-1-yl) | methyl | H | 459 |
| 52 | 3-(4-methyl-piperazin-1-yl) | 4-chloro-phenyl | H | 554/556 |
| 53 | 4-morpholino | propyl | H | 474 |
| 54 | 3-(4-methyl-piperazin-1-yl) | propyne-2-yl | H | 483 |
| 55 | 4-(4-methyl-piperazin-1-yl) | propyne-2-yl | H | 483 |
| 56 | 4-morpholino | isobutyl | H | 488 |
| 57 | 4-morpholino | ethyl | H | 460 |
| 58 | 3-morpholino | ethyl | H | 460 |
| 59 | 3-morpholino | propyl | H | 474 |
| 60 | 4-(4-methyl-piperazin-1-yl) | 2-chloro-phenyl | H | 554/556 |
| 61 | 3-(4-methyl-piperazin-1-yl) | 2-chloro-phenyl | H | 554/556 |
| 62 | 3-(4-methyl-piperazin-1-yl) | isobutyl | OCH₃ | 531 |
| 63 | 3-morpholino | isobutyl | H | 488 |
| 64 | 3-(4-methyl-piperazin-1-yl) | 3-trifluoro- | H | 589 |
| | | methyl-phenyl | | |
| 65 | 3-(4-methyl-piperazin-1-yl) | 2,3- | H | 549 |
| | | dimethylphenyl | | |
| 66 | 4-(4-methyl-piperazin-1-yl) | cyclohexyl | H | 527 |
| 67 | 3-(4-methyl-piperazin-1-yl) | cyclohexyl | H | 527 |
| 68 | 4-(4-methyl-piperazin-1-yl) | cis-2-methyl- | H | 541 |
| | | cyclohexyl | | |
| 69 | 3-(4-methyl-piperazin-1-yl) | cis-2-methyl- | H | 541 |
| | | cyclohexyl | | |
| 70 | 4-(4-methyl-piperazin-1-yl) | 1-cyclohexyl- | H | 555 |
| | | ethyl | | |
| 71 | 3-(4-methyl-piperazin-1-yl) | 1-cyclohexyl- | H | 555 |
| | | ethyl | | |
| 72 | 4-(4-methyl-piperazin-1-yl) | butyne-2-yl | H | 497 |
| 73 | 4-(4-methyl-piperazin-1-yl) | hexyl | H | 529 |
| 74 | 4-(4-methyl-piperazin-1-yl) | trans-2-methyl- | H | 541 |
| | | cyclohexyl | | |
| 75 | 3-(4-methyl-piperazin-1-yl) | trans-2-methyl- | H | 541 |
| | | cyclohexyl | | |
| 76 | 3-(4-methyl-piperazin-1-yl) | 2-methyl-butyl | H | 515 |
| 77 | 3-(4-methyl-piperazin-1-yl) | 4-ethylphenyl | H | 549 |
| 78 | 4-(4-methyl-piperazin-1-yl) | 4-ethylphenyl | H | 549 |
| 79 | 4-(4-methyl-piperazin-1-yl) | 4-tert-butylphenyl | H | 577 |
| 80 | 4-(4-methyl-piperazin-1-yl) | m-tolyl | H | 535 |
| 81 | 4-(4-methyl-piperazin-1-yl) | 2,3- | H | 549 |
| | | dimethylphenyl | | |
| 82 | 4-(4-methyl-piperazin-1-yl) | 4-isopropylphenyl | H | 563 |
| 83 | 3-(4-methyl-piperazin-1-yl) | 4-isopropylphenyl | H | 563 |
| 84 | 4-(4-methyl-piperazin-1-yl) | 4-sec-butylphenyl | H | 577 |
| 85 | 3-(4-methyl-piperazin-1-yl) | 4-sec-butylphenyl | H | 577 |
| 86 | 4-(4-methyl-piperazin-1-yl) | o-tolyl | H | 535 |
| 87 | 3-(4-methyl-piperazin-1-yl) | o-tolyl | H | 535 |
| 88 | 4-(4-methyl-piperazin-1-yl) | 3-methylpentyl | H | 529 |
| 89 | 3-(4-methyl-piperazin-1-yl) | 3-methylpentyl | H | 529 |
| 90 | 4-(4-methyl-piperazin-1-yl) | cyclohexyl- | H | 541 |
| | | methyl | | |
| 91 | 3-(4-methyl-piperazin-1-yl) | cyclohexyl- | H | 541 |
| | | methyl | | |
| 92 | 4-(4-methyl-piperazin-1-yl) | 2-methyl-pentyl | H | 529 |
| 93 | 3-(4-methyl-piperazin-1-yl) | 2-methyl-pentyl | H | 529 |
| 94 | 4-(4-methyl-piperazin-1-yl) | 2,3-dimethylbutyl | H | 529 |
| 95 | 3-(4-methyl-piperazin-1-yl) | 2,3-dimethylbutyl | H | 529 |
| 96 | 3-(4-methyl-piperazin-1-yl) | m-tolyl | H | 535 |
| 97 | 3-(4-methyl-piperazin-1-yl) | 4-tert-butylphenyl | H | 577 |
| 98 | 4-(4-methyl-piperazin-1-yl) | cyclopentyl- | H | 527 |
| | | methyl | | |
| 99 | 4-(4-methyl-piperazin-1-yl) | 1-cyclopentyl- | H | 541 |
| | | ethyl | | |
| 100 | 3-(4-methyl-piperazin-1-yl) | cyclopentyl- | H | 527 |
| | | methyl | | |
| 101 | 4-(4-methyl-piperazin-1-yl) | trans-2-methyl- | H | 527 |
| | | cyclopentyl | | |
| 102 | 3-(4-methyl-piperazin-1-yl) | trans-2-methyl- | H | 527 |
| | | cyclopentyl | | |
| 103 | 4-(4-methyl-piperazin-1-yl) | 4-tert-butyl- | H | 583 |
| | | cyclohexyl | | |
| 104 | 3-(4-methyl-piperazin-1-yl) | 4-tert-butyl- | H | 583 |
| | | cyclohexyl | | |
| 105 | 4-(4-methyl-piperazin-1-yl) | cyclobutyl-methyl | H | 513 |
| 106 | 3-(4-methyl-piperazin-1-yl) | cyclopentyl | H | 513 |
| 107 | 4-(4-methyl-piperazin-1-yl) | cyclopropyl- | H | 499 |
| | | methyl | | |
| 108 | 3-(4-methyl-piperazin-1-yl) | cyclopropyl- | H | 499 |
| | | methyl | | |
| 109 | 3-(4-methyl-piperazin-1-yl) | cyclobutyl- | H | 513 |
| | | methyl | | |
| 110 | 4-(4-methyl-piperazin-1-yl) | 4-ethyl- | H | 555 |
| | | cyclohexyl | | |
| 111 | 3-(4-methyl-piperazin-1-yl) | 4-ethyl- | H | 555 |
| | | cyclohexyl | | |
| 112 | 4-(4-methyl-piperazin-1-yl) | cis-4-methyl- | H | 541 |
| | | cyclohexyl | | |
| 113 | 3-(4-methyl-piperazin-1-yl) | cis-4-methyl- | H | 541 |
| | | cyclohexyl | | |
| 114 | 3-(4-methyl-piperazin-1-yl) | 1-cyclopentyl- | H | 541 |
| | | ethyl | | |
| 115 | 4-(4-methyl-piperazin-1-yl) | cyclopentyl | H | 513 |
| 116 | 4-(4-methyl-piperazin-1-yl) | 2-methyl-butyl | H | 515 |
| 117 | 4-(4-methyl-piperazin-1-yl) | trans-4-methyl- | H | 541 |
| | | cyclohexyl | | |
| 118 | 3-(4-methyl-piperazin-1-yl) | trans-4-methyl- | H | 541 |
| | | cyclohexyl | | |
| 119 | 4-(4-methyl-piperazin-1-yl) | 2,6-dimethyl- | H | 555 |
| | | cyclohexyl | | |
| 120 | 3-(4-methyl-piperazin-1-yl) | 2,6-dimethyl- | H | 555 |
| | | cyclohexyl | | |

### EXAMPLE 121

### 1-(4-{7-Amino-3-[4-(4-methyl-piperazin-1-yl)-phenyl]-pyrazolo[1,5-a]pyrimidin-6-yl}-phenyl)-3-(2-chloro-phenyl)-urea

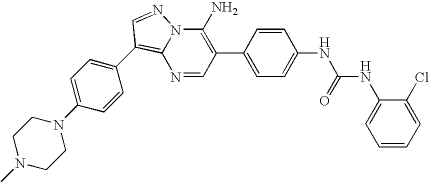

A suspension of 6-(4-amino-phenyl)-3-[4-(4-methyl-piperazin-1-yl)-phenyl]-pyrazolo[1,5-a]pyrimidin-7-ylamine (Ex. 4, 115 mg, 0.29 mM) in N-methyl-pyrrolidine (1.7 ml) is cooled to 0° C. and 4-nitrophenylchloroformate (68 mg, 0.34 mM) is added. The reaction mixture is stirred at 5° C. for 3.5 h, then 2-chloroaniline (89 mg, 0.70 mM) is added and the reaction mixture is stirred at 120° C. for 3 h. After cooling to room temperature, ethyl acetate and sat. NaHCO₃ solution are added and the layers are separated. The aqueous phase is extracted several times with ethyl acetate. The combined organic layers are washed with brine, dried over Na₂SO₄, and the solvent is removed in vacuo. The product is purified by preparative HPLC (H₂O with 0.1% TFA, 100%, 3 min; to H₂O/CH₃CN with 0.1% TFA, 1:9, inert 22 min; H₂O/CH₃CN with 0.1% TFA, 1:9, 5 min) to give the desired product as yellow crystals, M⁺H⁺=553, 555.

By following the procedure of above Examples but using the appropriate starting materials, the compounds of formula X₂ may be prepared

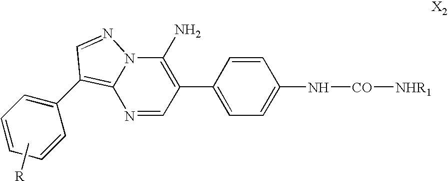

wherein R and R₁ have the significances as indicated in Table 2 below.

TABLE 2

| Ex | R | R₁ | M⁺H⁺ |
|---|---|---|---|
| 122 | 3-(4-methyl-piperazin-1-yl) | cyclohexyl | 526 |
| 123 | 3-(4-methyl-piperazin-1-yl) | 2-chloro-phenyl | 553/555 |
| 124 | 3-(4-methyl-piperazin-1-yl) | 2,3-dichloro-phenyl | 587/589 |
| 125 | 4-(4-methyl-piperazin-1-yl) | 2-chloro-phenyl | 553/555 |
| 126 | 3-(4-methyl-piperazin-1-yl) | p-tolyl | 534 |
| 127 | 3-(4-methyl-piperazin-1-yl) | 4-fluoro-phenyl | 538 |
| 128 | 3-(4-methyl-piperazin-1-yl) | ethyl | 472 |
| 129 | 4-(4-methyl-piperazin-1-yl) | ethyl | 472 |
| 130 | 3-(4-methyl-piperazin-1-yl) | cyclopentyl | 512 |
| 131 | 4-(4-methyl-piperazin-1-yl) | cyclopentyl | 512 |
| 132 | 4-(4-methyl-piperazin-1-yl) | 2-methylcyclohexyl | 540 |
| 133 | 4-(4-methyl-piperazin-1-yl) | (R)-1-cyclohexyl-ethyl | 554 |
| 134 | 3-(4-methyl-piperazin-1-yl) | (R)-1-cyclohexyl-ethyl | 554 |
| 135 | 3-(4-methyl-piperazin-1-yl) | cyclohexylmethyl | 540 |
| 136 | 4-(4-methyl-piperazin-1-yl) | cyclohexylmethyl | 540 |
| 137 | 3-(4-methyl-piperazin-1-yl) | (S)-1-cyclohexyl-ethyl | 554 |
| 138 | 3-(4-methyl-piperazin-1-yl) | 4-n-propyl-phenyl | 562 |
| 139 | 4-(4-methyl-piperazin-1-yl) | 4-n-propyl-phenyl | 562 |
| 140 | 3-(4-methyl-piperazin-1-yl) | 4-ethylphenyl | 548 |
| 141 | 3-(4-methyl-piperazin-1-yl) | 2-methylcyclohexyl | 540 |
| 142 | 4-(4-methyl-piperazin-1-yl) | 4-ethoxyphenyl | 564 |
| 143 | 4-(4-methyl-piperazin-1-yl) | 4-i-propyl-phenyl | 562 |
| 144 | 3-(4-methyl-piperazin-1-yl) | 4-i-propyl-phenyl | 562 |
| 145 | 4-(4-methyl-piperazin-1-yl) | 3,4-dimethylphenyl | 548 |
| 146 | 3-(4-methyl-piperazin-1-yl) | 3,4-dimethylphenyl | 548 |
| 147 | 4-(4-methyl-piperazin-1-yl) | (S)-1-cyclohexyl-ethyl | 554 |
| 148 | 4-(4-methyl-piperazin-1-yl) | 4-ethylphenyl | 548 |
| 149 | 3-(4-methyl-piperazin-1-yl) | 4-ethoxyphenyl | 564 |
| 150 | 4-(4-methyl-piperazin-1-yl) | 4-trifluoromethyl-phenyl | 588 |
| 151 | 3-(4-methyl-piperazin-1-yl) | 4-trifluoromethyl-phenyl | 588 |
| 152 | 4-(4-methyl-piperazin-1-yl) | 4-dimethylaminophenyl | 563 |
| 153 | 3-(4-methyl-piperazin-1-yl) | 4-dimethylaminophenyl | 563 |
| 154 | 3-(4-methyl-piperazin-1-yl) | 4-piperidin-1-yl-phenyl | 603 |
| 155 | 4-(4-methyl-piperazin-1-yl) | (R)-1-indan-1-yl | 560 |
| 156 | 3-(4-methyl-piperazin-1-yl) | (R)-1-indan-1-yl | 560 |
| 157 | 4-(4-methyl-piperazin-1-yl) | 4-methoxyphenyl | 550 |
| 158 | 3-(4-methyl-piperazin-1-yl) | 4-methoxyphenyl | 550 |
| 159 | 4-(4-methyl-piperazin-1-yl) | (S)-1-indan-1-yl | 560 |
| 160 | 4-(4-methyl-piperazin-1-yl) | (R)-1-phenyl-ethyl | 548 |
| 161 | 3-(4-methyl-piperazin-1-yl) | (R)-1-phenyl-ethyl | 548 |
| 162 | 4-(4-methyl-piperazin-1-yl) | (R)-1-p-tolyl-ethyl | 562 |
| 163 | 3-(4-methyl-piperazin-1-yl) | (R)-1-p-tolyl-ethyl | 562 |
| 164 | 4-(4-methyl-piperazin-1-yl) | (R)-1-(1,2,3,4- | 574 |
| | | tetrahydro-naphthalen- | |
| | | 1-yl | |
| 165 | 3-(4-methyl-piperazin-1-yl) | (R)-1-(1,2,3,4- | 574 |
| | | tetrahydro-naphthalen- | |
| | | 1-yl | |
| 166 | 4-(4-methyl-piperazin-1-yl) | (S)-1-phenyl-ethyl | 548 |
| 167 | 4-(4-methyl-piperazin-1-yl) | (S)-1-(1,2,3,4- | 574 |
| | | tetrahydro-naphthalen- | |
| | | 1-yl | |
| 168 | 3-(4-methyl-piperazin-1-yl) | (S)-1-(1,2,3,4- | 574 |
| | | tetrahydro-naphthalen- | |
| | | 1-yl | |
| 169 | 4-(4-methyl-piperazin-1-yl) | cyclopropylmethyl | 498 |
| 170 | 3-(4-methyl-piperazin-1-yl) | (S)-1-phenyl-ethyl | 548 |
| 171 | 4-(4-methyl-piperazin-1-yl) | (S)-1-p-tolyl-ethyl | 562 |
| 172 | 3-(4-methyl-piperazin-1-yl) | (S)-1-p-tolyl-ethyl | 562 |
| 173 | 4-(4-methyl-piperazin-1-yl) | 2,2,6,6-tetramethyl- | 583 |
| | | piperidin-4-yl | |
| 174 | 3-(4-methyl-piperazin-1-yl) | 2,2,6,6-tetramethyl- | 583 |
| | | piperidin-4-yl | |
| 175 | 4-(4-methyl-piperazin-1-yl) | 3-morpholin-4-yl-propyl | 571 |
| 176 | 3-(4-methyl-piperazin-1-yl) | cyclopropylmethyl | 498 |
| 177 | 3-(4-methyl-piperazin-1-yl) | 3-morpholin-4-yl-propyl | 571 |
| 178 | 4-(4-methyl-piperazin-1-yl) | cyclopentylmethyl | 526 |
| 179 | 3-(4-methyl-piperazin-1-yl) | cyclopentylmethyl | 526 |
| 180 | 3-(4-methyl-piperazin-1-yl) | (S)-1-methylhexyl | 542 |
| 181 | 4-(4-methyl-piperazin-1-yl) | (S)-1-methylhexyl | 542 |
| 182 | 4-(4-methyl-piperazin-1-yl) | 2-cyclohex-1-enyl-ethyl | 552 |
| 183 | 3-(4-methyl-piperazin-1-yl) | 2-cyclohex-1-enyl-ethyl | 552 |
| 184 | 4-(4-methyl-piperazin-1-yl) | cycloheptyl | 540 |
| 185 | 3-(4-methyl-piperazin-1-yl) | cycloheptyl | 540 |
| 186 | 4-(4-methyl-piperazin-1-yl) | cyclopentylmethyl | 554 |
| 187 | 3-(4-methyl-piperazin-1-yl) | cyclopentylmethyl | 554 |
| 188 | 4-(4-methyl-piperazin-1-yl) | n-pentyl | 514 |
| 189 | 3-(4-methyl-piperazin-1-yl) | n-pentyl | 514 |
| 190 | 4-(4-methyl-piperazin-1-yl) | 2-cyclopentylethyl | 540 |
| 191 | 3-(4-methyl-piperazin-1-yl) | 2-cyclopentylethyl | 540 |
| 192 | 4-(4-methyl-piperazin-1-yl) | n-hexyl | 528 |
| 193 | 3-(4-methyl-piperazin-1-yl) | n-hexyl | 528 |
| 194 | 4-(4-methyl-piperazin-1-yl) | (R)-1-methylhexyl | 542 |
| 195 | 3-(4-methyl-piperazin-1-yl) | (R)-1-methylhexyl | 542 |
| 196 | 4-(4-methyl-piperazin-1-yl) | 2-cyclohexyl-ethyl | 554 |
| 197 | 3-(4-methyl-piperazin-1-yl) | 2-cyclohexyl-ethyl | 554 |
| 198 | 3-(4-methyl-piperazin-1-yl) | (R)-indan-1-yl | 560 |

By using the same procedure as for Example 121 but using the appropriate starting materials, the compounds with the formula X₂ is obtainable

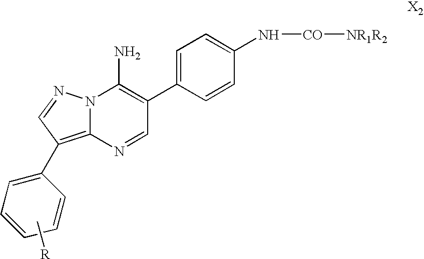

wherein R, and NR₁R₂ have the significances as indicated below.

| Example | R | NR₁R₂ | MH+ |
|---|---|---|---|
| 199 | 4-(4-methyl-piperazin-1-yl) | piperidine | 512 |
| 200 | 3-(4-methyl-piperazin-1-yl) | piperidine | 512 |
| 201 | 4-(4-methyl-piperazin-1-yl) | 1-methyl-1-p-tolyl-amine | 548 |
| 202 | 3-(4-methyl-piperazin-1-yl) | 1-methyl-1-p-tolyl-amine | 548 |
| 203 | 4-(4-methyl-piperazin-1-yl) | 1-cyclohexyl-1-methyl- | 540 |
| | | amine | |
| 204 | 3-(4-methyl-piperazin-1-yl) | 1-cyclohexyl-1-methyl- | 540 |
| | | amine | |

### EXAMPLE 205

### N-(4-{7-Amino-3-[4-(4-methyl-piperazin-1-yl)-phenyl]-pyrazolo[1,5-a]pyrimidin-6-yl}-phenyl)-butyramide

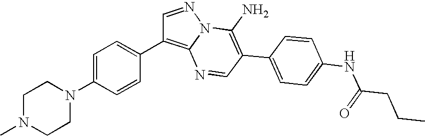

To a suspension of 6-(4-amino-phenyl)-3-[4-(4-methyl-piperazin-1-yl)-phenyl]-pyrazolo[1,5-a]pyrimidin-7-ylamine (Ex. 4, 112 mg, 0.28 mM) in pyridine/CH₂Cl₂ (1:1, 2 ml) buturyl chloride (37 mg, 0.35 mM) is added at room temperature and stirred for 1 h. Buturyl chloride (37 mg, 0.35 mM) is added again and the reaction mixture stirred for 1 h more at room temperature. Ethyl acetate and sat. NaHCO₃ solution are added and the layers are separated. The aqueous phase is extracted several times with ethyl acetate. The combined organic layers are washed with brine, dried over Na₂SO₄, and the solvent is removed in vacuo. The product is purified by preparative HPLC(H₂O with 0.1% TFA, 100%, 3 min; to H₂O/CH₃CN with 0.1% TFA, 1:9, inert 22 min; H₂O/CH₃CN with 0.1% TFA, 1:9, 5 min) to give the desired product as yellow crystals, M⁺H⁺=471.

By following the procedure of above Examples but using the appropriate starting materials, the compounds of formula X₃ may be prepared

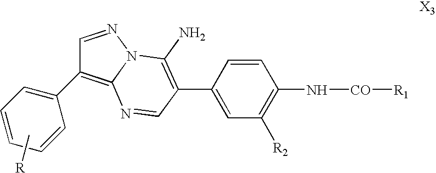

wherein R, R₁ and R₂ have the significances as indicated in Table 3 below.

TABLE 3

| Ex | R | R₁ | R₂ | M⁺H⁺ |
|---|---|---|---|---|
| 206 | 3-(4-methyl-piperazin-1-yl) | benzyl | H | 519 |
| 207 | 4-(4-methyl-piperazin-1-yl) | benzyl | H | 519 |
| 208 | 4-(4-methyl-piperazin-1-yl) | 1-methyl-indol- | OCH₃ | 588 |
| | | 2-yl | | |
| 209 | 3-(4-methyl-piperazin-1-yl) | 1-methyl-indol- | H | 558 |
| | | 2-yl | | |
| 210 | 3-(4-methyl-piperazin-1-yl) | 3-trifluoro- | H | 573 |
| | | methyl-phenyl | | |
| 211 | 3-(4-methyl-piperazin-1-yl) | 2-thienyl | H | 511 |
| 212 | 3-(4-methyl-piperazin-1-yl) | 3-chloro-phenyl | H | 538/540 |
| 213 | 3-(4-methyl-piperazin-1-yl) | 1-methyl-indol- | OCH₃ | 588 |
| | | 2-yl | | |
| 214 | 4-(4-methyl-piperazin-1-yl) | 2-thienyl | H | 511 |
| 215 | 3-(4-methyl-piperazin-1-yl) | 2-chloro-phenyl | H | 538/540 |
| 216 | 3-(4-methyl-piperazin-1-yl) | propyl | H | 471 |
| 217 | 4-(4-methyl-piperazin-1-yl) | propyl | H | 471 |
| 218 | 4-(4-methyl-piperazin-1-yl) | phenyl | H | 505 |
| 219 | 4-(4-methyl-piperazin-1-yl) | 2-furyl | H | 495 |
| 220 | 3-(4-methyl-piperazin-1-yl) | 2,3-dichloro- | H | 572/574 |
| | | phenyl | | |
| 221 | 3-(4-methyl-piperazin-1-yl) | 3-pyridyl | H | 506 |
| 222 | 3-(4-methyl-piperazin-1-yl) | 2-trifluoro- | H | 573 |
| | | methyl-phenyl | | |
| 223 | 3-(4-methyl-piperazin-1-yl) | 2,3-xylyl | H | 533 |
| 224 | 3-(4-methyl-piperazin-1-yl) | isobutyl | H | 485 |
| 225 | 4-(4-methyl-piperazin-1-yl) | 2,3-xylyl | H | 533 |
| 226 | 3-(4-methyl-piperazin-1-yl) | 2-tolyl | H | 519 |
| 227 | 4-(4-methyl-piperazin-1-yl) | 1-methyl-indol- | H | 558 |
| | | 2-yl | | |
| 228 | 3-(4-methyl-piperazin-1-yl) | 3-methoxybenzyl | H | 549 |
| 229 | 3-(4-methyl-piperazin-1-yl) | 3,4- | H | 579 |
| | | dimethoxybenzyl | | |
| 230 | 3-(4-methyl-piperazin-1-yl) | 4-methoxybenzyl | H | 549 |
| 231 | 4-(4-methyl-piperazin-1-yl) | 4-methoxybenzyl | H | 549 |
| 232 | 4-(4-methyl-piperazin-1-yl) | 3-methoxybenzyl | H | 549 |
| 233 | 4-(4-methyl-piperazin-1-yl) | 3,4- | H | 579 |
| | | dimethoxybenzyl | | |
| 234 | 3-(4-methyl-piperazin-1-yl) | 4-trifluoro- | H | 573 |
| | | methyl-phenyl | | |
| 235 | 4-(4-methyl-piperazin-1-yl) | 4-trifluoro- | H | 573 |
| | | methyl-phenyl | | |
| 236 | 4-(4-methyl-piperazin-1-yl) | 2-phenylethyl | H | 533 |
| 237 | 3-(4-methyl-piperazin-1-yl) | 2-phenylethyl | H | 533 |
| 238 | 3-(4-methyl-piperazin-1-yl) | 2,5- | H | 579 |
| | | dimethoxybenzyl | | |
| 239 | 4-(4-methyl-piperazin-1-yl) | 2,5- | H | 579 |
| | | dimethoxybenzyl | | |
| 240 | 3-(4-methyl-piperazin-1-yl) | 4-chlorophenyl | H | 539 |
| 241 | 4-(4-methyl-piperazin-1-yl) | 4-chlorophenyl | H | 539 |
| 242 | 4-(4-methyl-piperazin-1-yl) | 2-cyclohexyl- | H | 539 |
| | | ethyl | | |
| 243 | 3-(4-methyl-piperazin-1-yl) | 2-cyclohexyl- | H | 539 |
| | | ethyl | | |
| 244 | 4-(4-methyl-piperazin-1-yl) | cyclohexyl | H | 511 |
| 245 | 3-(4-methyl-piperazin-1-yl) | cyclohexyl | H | 511 |
| 246 | 4-(4-methyl-piperazin-1-yl) | 2-cyclopentyl- | H | 525 |
| | | ethyl | | |
| 247 | 3-(4-methyl-piperazin-1-yl) | 2-cyclopentyl- | H | 525 |
| | | ethyl | | |
| 248 | 4-(4-methyl-piperazin-1-yl) | cyclopentyl | H | 497 |
| 249 | 3-(4-methyl-piperazin-1-yl) | cyclopentyl | H | 497 |
| 250 | 4-(4-methyl-piperazin-1-yl) | thiophen-2-yl- | H | 525 |
| | | methyl | | |
| 251 | 3-(4-methyl-piperazin-1-yl) | thiophen-2-yl- | H | 525 |
| | | methyl | | |
| 252 | 4-(4-methyl-piperazin-1-yl) | 1-phenyl-propyl | H | 547 |
| 253 | 4-(4-methyl-piperazin-1-yl) | cyclopentyl- | H | 511 |
| | | methyl | | |
| 254 | 3-(4-methyl-piperazin-1-yl) | cyclopentyl- | H | 511 |
| | | methyl | | |
| 255 | 4-(4-methyl-piperazin-1-yl) | trans-2-phenyl- | H | 545 |
| | | cyclopropyl | | |
| 256 | 3-(4-methyl-piperazin-1-yl) | trans-2-phenyl- | H | 545 |
| | | cyclopropyl | | |
| 257 | 4-(4-methyl-piperazin-1-yl) | 1-phenoxy-ethyl | H | 549 |
| 258 | 3-(4-methyl-piperazin-1-yl) | 1-phenoxy-ethyl | H | 549 |
| 259 | 4-(4-methyl-piperazin-1-yl) | 4-t- | H | 567 |
| | | butylcyclohexyl | | |
| 260 | 3-(4-methyl-piperazin-1-yl) | 4-t- | H | 567 |
| | | butylcyclohexyl | | |
| 261 | 4-(4-methyl-piperazin-1-yl) | 2-methyl- | H | 525 |
| | | cyclohexyl | | |
| 262 | 3-(4-methyl-piperazin-1-yl) | 2-methyl- | H | 525 |
| | | cyclohexyl | | |
| 263 | 4-(4-methyl-piperazin-1-yl) | 2-phenyl-propyl | H | 547 |
| 264 | 3-(4-methyl-piperazin-1-yl) | 2-phenyl-propyl | H | 547 |
| 265 | 4-(4-methyl-piperazin-1-yl) | 2-methyl-benzyl | H | 533 |
| 266 | 3-(4-methyl-piperazin-1-yl) | 2-methyl-benzyl | H | 533 |
| 267 | 4-(4-methyl-piperazin-1-yl) | 3-methyl-benzyl | H | 533 |
| 268 | 3-(4-methyl-piperazin-1-yl) | 3-methyl-benzyl | H | 533 |
| 269 | 4-(4-methyl-piperazin-1-yl) | 4-methyl-benzyl | H | 533 |
| 270 | 3-(4-methyl-piperazin-1-yl) | 4-methyl-benzyl | H | 533 |
| 271 | 3-(4-methyl-piperazin-1-yl) | 1-phenyl-propyl | H | 547 |
| 272 | 4-(4-methyl-piperazin-1-yl) | 2,3- | H | 547 |
| | | dihydro- | | |
| | | benzofuran- | | |
| | | 2-yl | | |
| 273 | 4-(4-methyl-piperazin-1-yl) | benzofuran-2-yl | H | 545 |
| 274 | 4-(4-methyl-piperazin-1-yl) | n-hexyl | H | 513 |
| 275 | 3-(4-methyl-piperazin-1-yl) | n-hexyl | H | 513 |
| 276 | 4-(4-methyl-piperazin-1-yl) | n-pentyl | H | 499 |
| 277 | 3-(4-methyl-piperazin-1-yl) | n-pentyl | H | 499 |
| 278 | 4-(4-methyl-piperazin-1-yl) | n-heptyl | H | 527 |
| 279 | 3-(4-methyl-piperazin-1-yl) | n-heptyl | H | 527 |
| 280 | 4-(4-methyl-piperazin-1-yl) | chroman-3-yl | H | 561 |
| 281 | 3-(4-methyl-piperazin-1-yl) | chroman-3-yl | H | 561 |
| 282 | 3-(4-methyl-piperazin-1-yl) | 2,3-dihydro- | H | 547 |
| | | benzofuran-2-yl | | |
| 283 | 3-(4-methyl-piperazin-1-yl) | benzofuran-2-yl | H | 545 |
| 284 | 4-(4-methyl-piperazin-1-yl) | 1,2,3,4- | H | 559 |
| | | tetrahydro- | | |
| | | naphthalen-2-yl | | |
| 285 | 3-(4-methyl-piperazin-1-yl) | 1,2,3,4- | H | 559 |
| | | tetrahydro- | | |
| | | naphthalen-2-yl | | |
| 286 | 4-(4-methyl-piperazin-1-yl) | cyclohexyl- | H | 525 |
| | | methyl | | |
| 287 | 3-(4-methyl-piperazin-1-yl) | cyclohexyl- | H | 525 |
| | | methyl | | |
| 288 | 4-(4-methyl-piperazin-1-yl) | 1-ethyl-pentyl | H | 527 |
| 289 | 3-(4-methyl-piperazin-1-yl) | 1-ethyl-pentyl | H | 527 |
| 290 | 4-(4-methyl-piperazin-1-yl) | indan-2-yl | H | 545 |
| 291 | 3-(4-methyl-piperazin-1-yl) | indan-2-yl | H | 545 |
| 292 | 4-(4-methyl-piperazin-1-yl) | 3-cyclohexyl- | H | 553 |
| | | propyl | | |
| 293 | 3-(4-methyl-piperazin-1-yl) | 3-cyclohexyl- | H | 553 |
| | | propyl | | |
| 294 | 4-(4-methyl-piperazin-1-yl) | 2-cyclopropyl- | H | 497 |
| | | ethyl | | |
| 295 | 3-(4-methyl-piperazin-1-yl) | 2-cyclopropyl- | H | 497 |
| | | ethyl | | |
| 296 | 4-(4-methyl-piperazin-1-yl) | 3-methyl-1H- | H | 557 |
| | | inden-2-yl | | |
| 297 | 4-(4-methyl-piperazin-1-yl) | n-butyl | H | 485 |
| 298 | 4-(4-methyl-piperazin-1-yl) | 5-oxo-hexyl | H | 527 |
| 299 | 3-(4-methyl-piperazin-1-yl) | 5-oxo-hexyl | H | 527 |
| 300 | 3-(4-methyl-piperazin-1-yl) | n-butyl | H | 485 |
| 301 | 4-(4-methyl-piperazin-1-yl) | 4- | H | 539 |
| | | methyl- | | |
| | | cyclohexyl- | | |
| | | methyl | | |
| 302 | 3-(4-methyl-piperazin-1-yl) | 4- | H | 539 |
| | | methyl- | | |
| | | cyclohexyl- | | |
| | | methyl | | |
| 303 | 4-(4-methyl-piperazin-1-yl) | 2,4,4- | H | 541 |
| | | trimethylpentyl | | |
| 304 | 3-(4-methyl-piperazin-1-yl) | 2,4,4- | H | 541 |
| | | trimethylpentyl | | |
| 305 | 4-(4-methyl-piperazin-1-yl) | 2-tetrahydrofuran- | H | 527 |
| | | 2-yl-ethyl | | |
| 306 | 4-(4-methyl-piperazin-1-yl) | 4-oxopentyl | H | 513 |
| 307 | 3-(4-methyl-piperazin-1-yl) | 4-oxopentyl | H | 513 |
| 308 | 4-(4-methyl-piperazin-1-yl) | 1-methyl-pentyl | H | 513 |
| 309 | 3-(4-methyl-piperazin-1-yl) | 1-methyl-pentyl | H | 513 |
| 310 | 3-(4-methyl-piperazin-1-yl) | 2-tetrahydrofuran- | H | 527 |
| | | 2-yl-ethyl | | |
| 311 | 3-(4-methyl-piperazin-1-yl) | 3-methyl-1H- | H | 557 |
| | | inden-2-yl | | |
| 312 | 4-(4-methyl-piperazin-1-yl) | 3-phenyl-propyl | H | 547 |
| 313 | 3-(4-methyl-piperazin-1-yl) | 3-phenyl-propyl | H | 547 |
| 314 | 3-(4-methyl-piperazin-1-yl) | benzofuran- | H | 559 |
| | | 2-yl-methyl | | |
| 315 | 4-(4-methyl-piperazin-1-yl) | 3-methyl-pentyl | H | 513 |
| 316 | 3-(4-methyl-piperazin-1-yl) | 3-methyl-pentyl | H | 513 |
| 317 | 4-(4-methyl-piperazin-1-yl) | benzofuran-3- | H | 559 |
| | | yl-methyl | | |

### Generic Procedure

### Parallel Synthesis of N-(3-{7-Amino-3-[3-(4-methyl-piperazin-1-yl)-phenyl]-pyrazolo[1,5-a]pyrimidin-6-yl}-phenyl)-sulfonamides

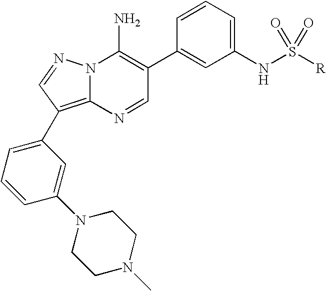

To an array of glass tubes is added 6-(3-amino-phenyl)-3-[3-(4-methyl-piperazin-1-yl)-phenyl]-pyrazolo[1,5-a]pyrimidin-7-ylamine (50 mg, 0.40 mmol, 1 eq.), pyridine (0.8 ml) and one of the 17 sulfonyl chlorides (0.80 mmol, 2 eq.) in each tube. All tubes are flushed with argon and closed. The resulting reaction mixtures are stirred at room temperature for 60 hours. Then a solution of 33% of methylamine in ethanol (30.6 μl) is added to each tube and stirring is continued at room temperature for 1 hour. The solvents are evaporated and the resulting residues are individually re-dissolved in a mixture of methanol (3 ml), acetonitrile (0.5 ml) and two drops of water containing 1% of TFA. Each solution is individually filtered over a 0.45 μm PTFA membrane and the filtrates are then purified by a preparative HPLC/MS procedure.

### EXAMPLE 318

### 4-(3-{7-Amino-6-[3-(2-chloro-benzenesulfonylamino)-phenyl]-pyrazolo[1,5-a]pyrimidin-3-yl}-phenyl)-1-benzyl-1-methyl-piperazin-1-ium bromide

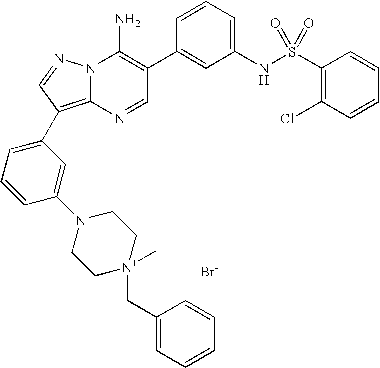

To a glass tube is added N-(3-{7-amino-3-[3-(4-methyl-piperazin-1-yl)-phenyl]-pyrazolo[1,5-a]pyrimidin-6-yl}phenyl)-2-chloro-benzenesulfonamide (30 mg, 0.052 mmol, 1 eq.), K₂CO₃ (11.4 mg, 0.082 mmol, 1.6 eq.) and a solution of benzyl bromide (60 μl, 0.031 mmol, 0.6 eq.) in DMF (0.3 ml). The reaction mixture is stirred at 8° C. during 10 minutes, followed by addition of a solution of benzyl bromide (50 μl, 0.026 mmol, 0.5 eq.) in DMF (0.2 ml). Stirring is continued for 1 h 30 at 8° C. and then for 30 minutes at room temperature. The reaction mixture is diluted with DMF (2 ml), filtered over a 0.45 μm PTFA membrane and the filtrate is purified by a preparative HPLC/MS procedure. Freeze drying of the pooled fractions give a white powder. M⁺H⁺ 664.3.

By following the procedure of above Examples but using the appropriate starting materials, the compounds of formula X₄ may be prepared

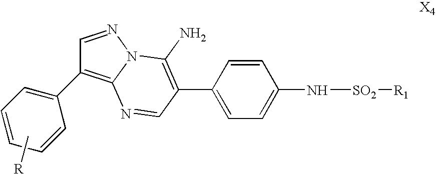

wherein R and R₁ have the significances as indicated in Table 4 below.

TABLE 4

| Ex | R | R₁ | M⁺H⁺ |
|---|---|---|---|
| 319 | 3-(4-methyl- | 2-chloro-phenyl | 574 |
| | piperazin-1-yl) | | |
| 320 | 3-(4-methyl- | 3-cyano-phenyl | 565 |
| | piperazin-1-yl) | | |
| 321 | 3-(4-methyl- | 3-trifluoromethyl- | 608 |
| | piperazin-1-yl) | phenyl | |
| 322 | 3-(4-methyl- | 2-trifluoromethyl- | 608 |
| | piperazin-1-yl) | phenyl | |
| 323 | 3-(4-methyl- | 3-thienyl | 546 |
| | piperazin-1-yl) | | |
| 324 | 3-(4-methyl- | propyl | 506 |
| | piperazin-1-yl) | | |
| 325 | 3-(4-methyl- | 1,2-dimethyl-4- | 558 |
| | piperazin-1-yl) | imidazolyl | |
| 326 | 3-(4-methyl- | 4-methoxy-phenyl | 570 |
| | piperazin-1-yl) | | |
| 327 | 3-(4-methyl- | 4-methyl-benzyl | 568 |
| | piperazin-1-yl) | | |
| 328 | 3-(4-methyl- | 4-tolyl | 554 |
| | piperazin-1-yl) | | |
| 329 | 3-(4-methyl- | 4-pyrazolyl-phenyl | 606 |
| | piperazin-1-yl) | | |
| 330 | 3-(4-methyl- | phenethyl | 568 |
| | piperazin-1-yl) | | |
| 331 | 3-(4-methyl- | 4-ethyl-phenyl | 568 |
| | piperazin-1-yl) | | |
| 332 | 3-(4-methyl- | benzothiazol-6-yl | 597 |
| | piperazin-1-yl) | | |
| 333 | 3-(4-methyl- | 2,4-dimethyl-5- | 575 |
| | piperazin-1-yl) | thiazolyl | |

### EXAMPLE 334

### [4-(7-Amino-3-{3-[4-((S)-2-amino-3-methyl-butyryl)-piperazin-1-yl]-phenyl}-pyrazolo[1,5-a]pyrimidin-6-yl)-phenyl]-carbamic acid isobutyl ester

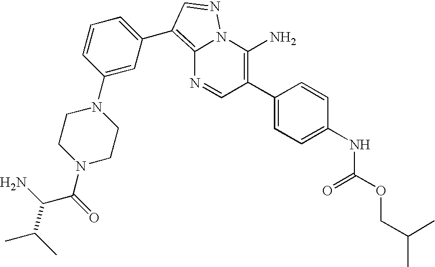

[4-(7-Amino-3{3-[4-((S)-2-benzyloxycarbonylamino-3-methyl-butyryl)-piperazin-1-yl]-phenyl}-pyrazolo[1,5-a]pyrimidin-6-yl)-phenyl]-carbamic acid isobutyl ester (54 mg, 0.075 mMol) is dissolved in tetrahydrofuran/methanol 1:1. 4 mg Palladium (10%) on carbon are added and the mixture is hydrogenated for 65 hours at room temperature under normal pressure. The reaction mixture is filtered, the solvent is removed in vacuo and the product is isolated by lyophilization from tert. Butanol (M⁺H⁺ 585.8, white powder).

The starting material can be prepared as follows:

### a) 4-(3-Cyanomethyl-phenyl)-piperazine-1-carboxylic acid benzyl ester

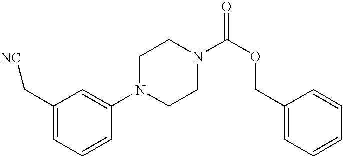

(3-bromo-phenyl)-acetonitrile (5.1 g, 25.5 mMol) is dissolved in dimethoxyethane (54 ml). After addition of piperazine-1-carboxylic acid benzylester (11.4 g, 51 mMol), potassium phosphate (10.8 g, 51 mMol), (2-biphenyl)di-tert butylphosphine (2.28 g, 7.6 mMol) and palladium-II-acetate (573 mg, 2.55 mMol) the mixture is refluxed for 20 hours. After cooling to room temperature the mixture is filtered and the brown filtrate is evaporated in vacuo to give a brown oil. The crude mixture is separated by flash chromatography (gradient of ethyl acetate/hexane 1:9 to 1:1) yielding the pure product as a dark yellow oil (M⁺H⁺ 336.2).

### b) 4-[3-(1-Cyano-2-oxo-ethyl)-phenyl]-piperazine-1-carboxylic acid benzyl ester

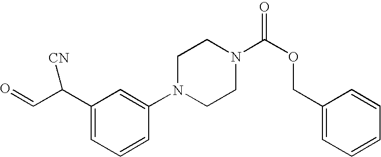

4-(3-Cyanomethyl-phenyl)-piperazine-1-carboxylic acid benzyl ester (5.9 g, 17.6 mMol) is dissolved in toluene (59 ml). After addition of ethyl formate (2.122 ml, 26.4 mMol) and sodium methylate (1.425 g, 26.4 mMol) the mixture is stirred at 38° C. for 3 hours. The original slight yellow suspension turns brown. The mixture is evaporated to dryness, the residue is treated with toluene (50 ml) and evaporated in vacuo three times. The crude product (M⁺H⁺ 364.2) is used without purification in the next step.

### c) 4-[3-(5-Amino-1H-pyrazol-4-yl)-phenyl]-piperazine-1-carboxylic acid benzyl ester

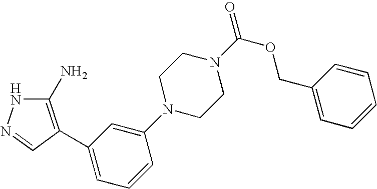

4-[3-(1-Cyano-2-oxo-ethyl)-phenyl]-piperazine-1-carboxylic acid benzyl ester (500 mg, 1.38 mMol) is dissolved in toluene (3 ml) and treated with acetic acid (0.24 ml, 4.13 mMol). The grey-brown suspension becomes beige. The reaction temperature rises to 30° C. Hydrazine monohydrate (138 mg, 2.75 mMol) is added (reaction temperature rises to 40° C.). The mixture is heated to reflux for 1.5 hours and then cooled to room temperature. Saturated aqueous sodium carbonate (20 ml) and dichloromethane (30 ml) are added. The layers are separated, the organic layer is washed with water, dried over sodium sulfate and evaporated in vacuo. The crude mixture is separated by flash chromatography (gradient of dichloromethane/methanol 1:0 to 7:3). The product is received as slightly yellow amorphous solid (M⁺H⁺ 378.3).

### d) 4-{3-[7-Amino-6-(4-isobutoxycarbonylamino-phenyl)-pyrazolo[1,5-a]pyrimidin-3-yl]-phenyl}-piperazine-1-carboxylic acid benzyl ester

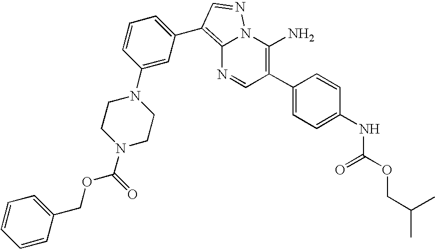

4-[3-(5-amino-1H-pyrazol-4-yl)-phenyl]-piperazine-1-carboxylic acid benzyl ester (1.52 g, 4.04 mMol), [4-((Z)-1-cyano-2-dimethylamino-vinyl)-phenyl]-carbamic acid isobutyl ester (1.16 g, 4.04 mMol) are dissolved in ethanolic HCl (1.25M, 8.4 ml) and 7.4 ml acetic acid. The mixture is heated to reflux for 16 hours, cooled to room temperature, poured into saturated aqueous sodium carbonate (50 ml) and extracted with dichloromethane. The organic layer is dried over sodium sulfate and evaporated in vacuo. The crude mixture is separated by flash chromatography (gradient cyclohexane/ethyl acetate 9:1 to 1:1. Evaporation of the corresponding fractions yields the desired product as yellow amorphous solid M⁺H⁺ 620.3).

### e) {4-[7-Amino-3-(3-piperazin-1-yl-phenyl)-pyrazolo[1,5-a]pyrimidin-6-yl]-phenyl}-carbamic acid isobutyl ester

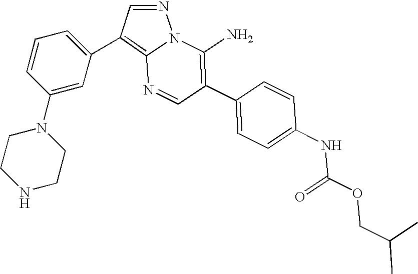

4-{3-[7-Amino-6-(4-isobutoxycarbonylamino-phenyl)-pyrazolo[1,5-a]pyrimidin-3-yl]-phenyl}-piperazine-1-carboxylic acid benzyl ester (1.5 g, 2.4 mMol) are dissolved in methanol (24 ml). After addition of palladium (10%, 257 mg) on carbon the mixture is hydrogenated at room temperature under normal pressure until all starting material is used up. The reaction mixture is filtered and evaporated in vacuo yielding the product as a slightly yellow amorphous solid (M⁺H⁺ 486.2).

### f) [4-(7-Amino-3-{3-[4-((S)-2-benzyloxycarbonylamino-3-methyl-butyryl)-piperazin-1-yl]-phenyl}-pyrazolo[1,5-a]pyrimidin-6-yl)-phenyl]-carbamic acid isobutyl ester

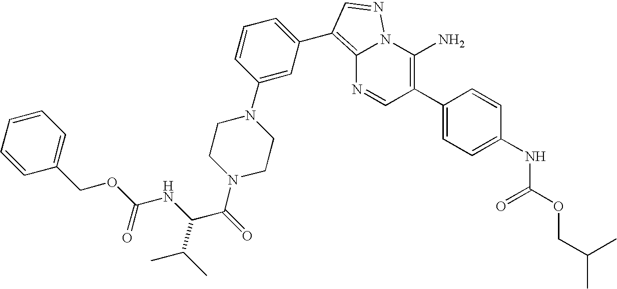

{4-[7-amino-3-(3-piperazin-1-yl-phenyl)-pyrazolo[1,5-a]pyrimidin-6-yl]-phenyl}-carbamic acid isobutyl ester (51 mg, 0.1 mMol), Z-(L)-valine (33 mg, 0.13 mMol) and N-hydroxybenzotriazol HOBt (18 mg, 0.13 mMol), triethylamine (0.019 ml, 0.13 mMol) are dissolved in 4 ml tetrahydrofuran, cooled to 0° C. and then treated with N-(3-dimethylaminopropyl)-N [symbol] ethyl-carbodiimide (0.024 ml, 0.13 mMol). The reaction mixture is stirred at room temperature for 20 hours and then evaporated in vacuo. The residue is treated with saturated aqueous potassium carbonate and extracted with ethyl acetate. The organic layer is washed with water, dried over sodium sulfate and evaporated in vacuo. The crude product is separated by flash chromatography (dichloromethane/methanol 1:0, gradient to 93:7). The product is isolated by lyophilization from tert. butanol (M⁺H⁺ 719.7, white powder).

### EXAMPLE 335

### [4-((Z)-1-Cyano-2-dimethylamino-vinyl)-phenyl]-carbamic acid isobutyl ester

### a) (4-Cyanomethyl-phenyl)-carbamic acid isobutyl ester

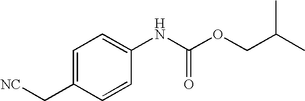

(4-amino-phenyl)-acetonitrile (1.33 g, 9.8 mMol) is dissolved in pyridine (21 ml). Isobutyl chloroformate (1.5 g, 10.8 mMol) is added and the mixture is stirred at room temperature for 1 hour and then at 60° C. for 1.5 hours. The reaction mixture is evaporated under reduced pressure. The crude mixture is separated by flash chromatography (gradient ethyl acetate/hexane 1:9 to 3:7) yielding the product that solidifies overnight at room temperature. The product in treated with cyclohexane and warmed to 50° C. for 30 minutes. Filtering and drying yields the product as yellow solid (M⁺H⁺ 233.1).

### b) [4-((Z)-1-Cyano-2-dimethylamino-vinyl)-phenyl]-carbamic acid isobutyl ester

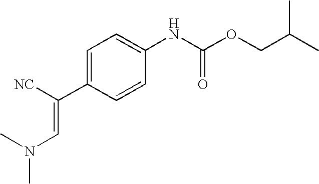

(4-cyanomethyl-phenyl)-carbamic acid isobutyl ester (1.79 g, 7.7 mMol) is dissolved in toluene (16 ml). After addition of N,N-dimethylformamide-dimethylacetal (1.84 g, 15 mMol) the mixture is refluxed for two hours. Additional N,N-dimethylformamide-dimethylacetal (1 g) is added and the reaction mixture is refluxed overnight (total reaction time 20 hours). Cooling to room temperature yields a brown suspension which is diluted with ethyl acetate (200 ml) and then evaporated in vacuo to give a brown solid. The crude mixture is separated by flash chromatography (gradient ethyl acetate/hexane 1:9 to 1:1) providing the product as orange solid (M⁺H⁺ 288.1).

By following the procedure of above Examples but using the appropriate starting materials, the compounds of formula X₅ may be prepared

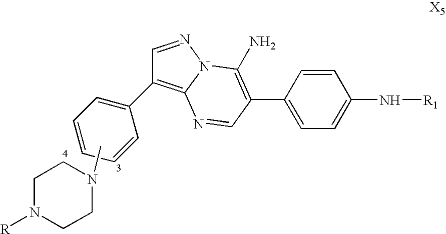

wherein R and R₁ have the significances as indicated in Table 5 below.

TABLE 5

| | Position | | | |
|---|---|---|---|---|
| | on the | | | |
| Ex | phenyl | R | R₁ | M⁺H⁺ |
| 336 | 4 | H | CO—O- | 487 |
| | | | isobutyl | |
| 337 | 4 | 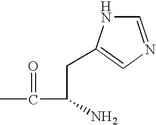 | CO—O- isobutyl | 623 |
| 338 | 4 | CO—CH₂—NHCH₃ | CO—O- | 557 |
| | | | isobutyl | |
| 339 | 4 | 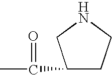 | CO—O- isobutyl | 584 |
| 340 | 4 | 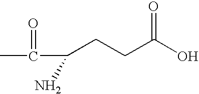 | CO—O- isobutyl | 616 |
| 341 | 3 | H | CO—O- | 486 |
| | | | isobutyl | |
| 342 | 3 | CO—CH₂—NH₂ | CO—O- | 543 |
| | | | isobutyl | |
| 343 | 3 | 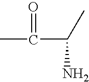 | CO—O- isobutyl | 558 |
| 344 | 3 | 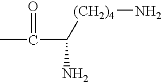 | CO—O- isobutyl | 615 |
| 345 | 3 | 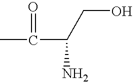 | CO—O- isobutyl | 574 |
| 346 | 3 | 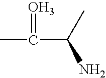 | CO—O- isobutyl | 558 |
| 347 | 4 | 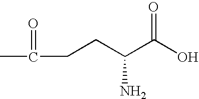 | CO—O- isobutyl | 616 |
| 348 | 3 | 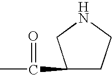 | CO—O- isobutyl | 584 |
| 349 | 3 | 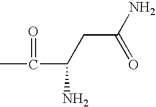 | CO—O- isobutyl | 600 |
| 350 | 3 | CO—(CH₂)₃—NH₂ | CO—O- | 572 |
| | | | isobutyl | |
| 351 | 3 | 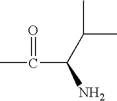 | CO—O- isobutyl | 586 |
| 352 | 3 | 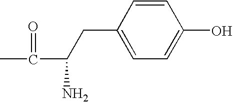 | CO—O- isobutyl | 650 |
| 353 | 3 | 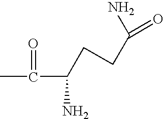 | CO—O- isobutyl | 614 |
| 354 | 4 | 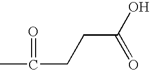 | CO—O- isobutyl | 587 |
| 355 | 3 | 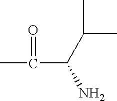 | CO—O- isobutyl | 586 |
| 356 | 4 | CO—(CH₂)₃—NH₂ | CO—O- | 572 |
| | | | isobutyl | |
| 357 | 3 | CH₃ | SO₂- | 591 |
| | | | napthyl | |
| 358 | 3 | 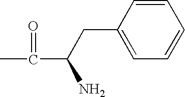 | CO—O- isobutyl | 634 |
| 359 | 3 | CO—CH₃ | SO₂-2- | 602 |
| | | | chloro- | |
| | | | phenyl | |
| 360 | 3 | CO—CH₂-3-pyridyl | CO—O- | 606 |
| | | | isobutyl | |
| 361 | 3 | CO-3-pyridyl | CO—O- | 592 |
| | | | isobutyl | |
| 362 | 3 | CO-4-imidyzolyl | CO—O- | 581 |
| | | | isobutyl | |
| 363 | 3 | 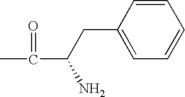 | CO—O- isobutyl | 634 |
| 364 | 3 | CO-4-pyridyl | CO—O- isobutyl | 591 |
| 365 | 3 | 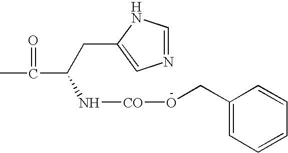 | CO—O- isobutyl | 758 |
| 366 | 3 | CO-2-pyridyl | CO—O- | 592 |
| | | | isobutyl | |
| 367 | 3 | CH₃ | CO—O- | 532 |
| | | | isobutyl | |
| 368 | 3 | 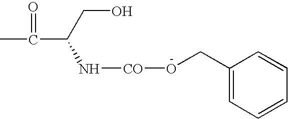 | CO—O- isobutyl | 708 |
| 369 | 3 | SO₂—CH₃ | CO—O- | 564 |
| | | | isobutyl | |
| 370 | 3 | 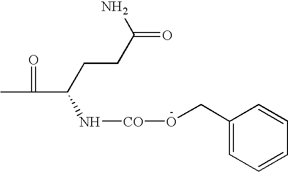 | CO—O- isobutyl | 749 |
| 371 | 3 | 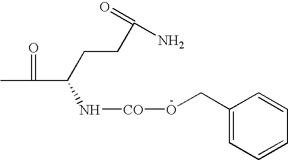 | CO—O- isobutyl | 735 |
| 372 | 3 | CH₃ | SO₂-ethyl | 493 |
| 373 | 3 | H | H | 384 |
| 374 | 3 | CH₃ | 3-methyl- | 518 |
| | | | benzoyl | |
| 375 | 3 | 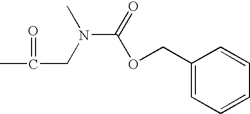 | CO—O- isobutyl | 691 |
| 376 | 3 | 4-amino-benzoyl | CO—O- | 605 |
| | | | isobutyl | |
| 377 | 3 | 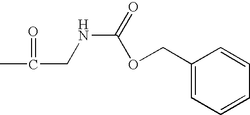 | CO—O- isobutyl | 677 |
| 378 | 4 | CH₃ | N(CH₃)—SO₂- | 588 |
| | | | 2-chloro- | |
| | | | phenyl | |
| 379 | 3 | phenylacetyl | CO—O- | 605 |
| | | | isobutyl | |
| 380 | 3 | 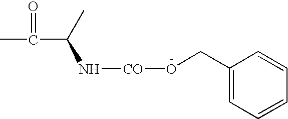 | CO—O- isobutyl | 692 |
| 381 | 3 | 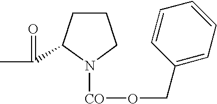 | CO—O- isobutyl | 718 |
| 382 | 3 | CH₃ | 4-fluoro- | 523 |
| | | | benzoyl | |
| 383 | 3 | benzoyl | CO—O- | 590 |
| | | | isobutyl | |
| 384 | 3 | 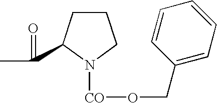 | CO—O- isobutyl | 718 |
| 385 | 3 | trifluoro-acetyl | CO—O- | 582 |
| | | | isobutyl | |
| 386 | 3 | 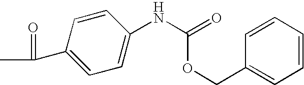 | CO—O- isobutyl | 739 |
| 387 | 4 | H | H | 458.2 |
| 388 | 4 | 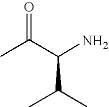 | —COO- isobutyl | 585.4 |

Following compounds may be obtained by using the procedure as disclosed above and using the appropriate starting materials:

### EXAMPLES 389 AND 390

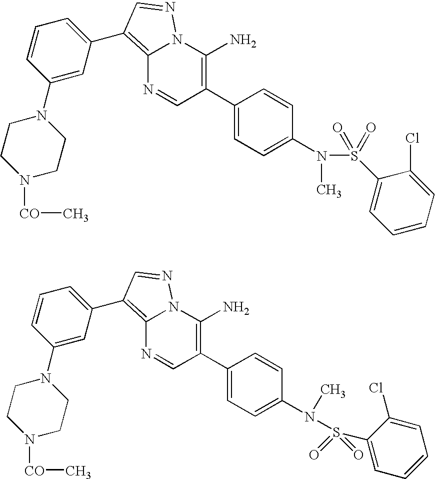

### EXAMPLE 391

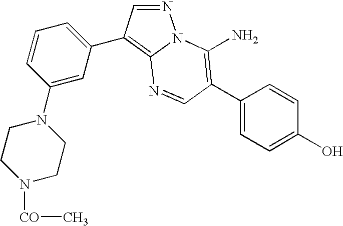

### EXAMPLE 392

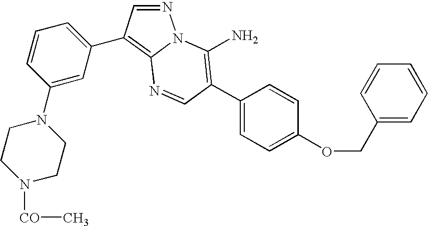

### EXAMPLE 393

### [4-(7-amino-3-{4[4-4(1-methyl-piperidin-4-yl)-piperazin-1-yl]-phenyl}-pyrazolo[1,5-a]pyrimidin-6-yl)-phenyl]-carbamic acid isobutyl ester

### A) {4-[4-(1-Methyl-piperidin-4-yl)-piperazin-1-yl]-phenyl}-acetonitrile

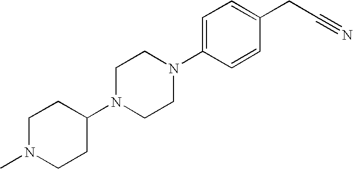

To a mixture of (4-bromo-phenyl)-acetonitrile (196 mg, 1 mmol), K₃PO₄ (318 mg, 1.5 mmol), 1-(1-methyl-piperidin-4-yl)-piperazine (220 mg, 1.2 mmol), (2-biphenyl)di-tert-butylphosphine (45 mg, 0.15 mmol) in 1,2-dimethoxyethane (3 ml) is added under argon atmosphere palladium(II) acetate (22 mg, 0.1 mmol). The mixture is shaken under argon in a tightly closed flask for 20 h at 90° C. After cooling to room temperature, H₂O and ethyl acetate are added and the mixture filtered through a pad of celite. The aqueous layer is separated and extracted twice with ethyl acetate. The combined organic layers are washed with H₂O, dried over Na₂SO₄. The solvent is removed in vacuo and the residue purified by preparative HPLC (YMC-Pack Pro C18 column; 10-100% CH₃CN+0.1% CF₃COOH/H₂O+0.1% CF₃COOH within 20 min, flow 20 ml/min) to give the desired product as a solid, [M+H]⁺=299.2; t_{R} (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 2.63 min.

### B) Sodium 2-cyano-2-{4-[4-(1-methyl-piperidin-4-yl)-piperazin-1-yl]-phenyl}-ethenolate

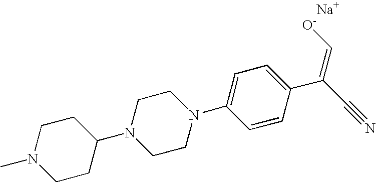

Sodium (345 mg, 15 mmol) is dissolved in ethanol (25 ml) at 50° C. After cooling to room temperature {4-[4-(1-methyl-piperidin-4-yl)-piperazin-1-yl]-phenyl}-acetonitrile (3.0 g, 10 mmol) and ethyl formate (1.2 ml, 15 mmol) and the reaction mixture stirred at 60° C. for 2 h. After cooling to room temperature, diethylether is added, the precipitate filtered off, washed with diethylether and dried in vacuo to afford the product as a dark brown solid. [M−H]⁻=325.3; t_{R} (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 2.35 min.

### C) 4-{4-[4-(1-Methyl-piperidin-4-yl)-piperazin-1-yl]-phenyl}-2H-pyrazol-3-ylamine

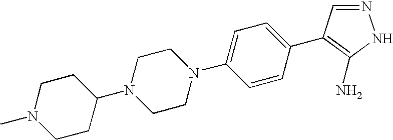

A mixture of the compound of Ex 393B (2.4 g, 6.9 mmol), hydrazine monohydrate (0.95 ml, 19.5 mmol) and acetic acid (30 ml) is stirred at 125° C. for 2 h. After cooling to room temperature, H₂O (60 ml) and conc. HCl (6 ml) are added and the mixture is stirred for 1 h at reflux temperature. The mixture is cooled to room temperature, basified with conc. NH₄OH solution, diluted with H₂O and the aqueous layer extracted twice with CH₂Cl₂. The organic extracts are discarded and the aqueous layer extracted twice with n-butanol. The combined butanol layers are evaporated in vacuo and the residue evaporated with toluene to give the product as a dark brown solid. [M+H]⁺=341.3; t_{R} (HPLC, CC 125/4 Nucleosil 100-5 C18 AB column; 0-100% CH₃CN+0.1% CF₃COOH/H₂O+0.1% CF₃COOH for 8 min, flow 1.5 ml/min): 2.75 min.

### D) 3-{4-[4-(1-Methyl-piperidin-4-yl)-piperazin-1-yl]-phenyl}-6-(4-nitro-phenyl)-pyrazolo[1,5-a]pyrimidin-7-ylamine, hydrochloride

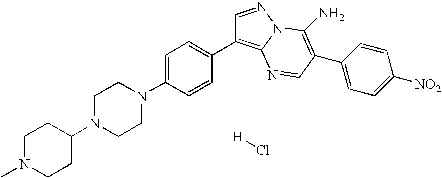

A mixture of the Compound of Ex 393C (272.4 mg, 0.8 mmol), 3-dimethylamino-2-(4-nitro-phenyl)-acrylonitrile (173.8 mg, 0.8 mmol), acetic acid (3 ml), ethanol (5 ml) and ˜1.25 M HCl in ethanol (2.55 ml, ˜3.2 mmol) is stirred at 85° C. for 18 h. After cooling to room temperature, the reaction mixture is filtered, the residue washed with ethanol and diethyl ether and dried in vacuo at 60° C. to yield the product as a dark brown solid. [M+H]⁺=513.2; t_{R} (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 2.95 min.

### E) 6-(4-Amino-phenyl)-3{4-[4-(1-methyl-piperidin-4-yl)-piperazin-1-yl]-phenyl}-pyrazolo[1,5-a]pyrimidin-7-ylamine, hydrochloride

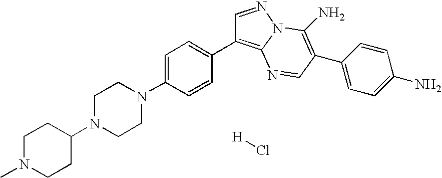

A mixture of the compound of Example 393D (316.9 mg, 0.58 mmol), DMF (12 ml), H₂O (18 ml) and Pd/C 10% (100 mg) is hydrogenated at room temperature for 16 h (hydrogen pressure ˜2 bar). The reaction mixture is filtered through a pad of celite, the residue washed with DMF and H₂O and the filtrate evaporated in vacuo to yield the crude product as a dark grey solid. For the analysis, part of the crude product is purified by preparative HPLC(YMC-Pack Pro C18 column; 0-100% CH₃CN+0.1% CF₃COOH/H₂O+0.1% CF₃COOH within 20 min, flow 20 ml/min) to yield the desired product as a brown solid, [M+H]⁺=483.3; t_{R} (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 2.17 min.

### F) [4-(7-Amino-3-{4-[4-(1-methyl-piperidin-4-yl)-piperazin-1-yl]-phenyl}-pyrazolo[1,5-a]pyrimidin-6-yl)-phenyl]-carbamic acid isobutyl ester

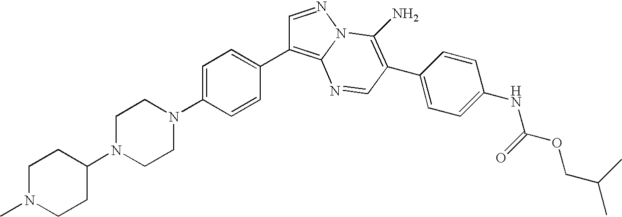

To a stirred mixture of the compound of Ex. 393E (96.5 mg, 0.18 mmol), DMF (2 ml) and pyridine (3 ml) is added isobutyl chloroformate (28.4 μl, 0.22 mmol). After 75 min at room temperature, a second portion of isobutyl chloroformate (28.4 μl, 0.22 mmol) is added and stirring is continued for 16 h. The reaction mixture is evaporated in vacuo and the residue distributed between 2N NaOH solution and ethyl acetate. The ethyl acetate extract is separated and the aqueous layer extracted twice with ethyl acetate. The combined organic layers are washed with brine, dried over Na₂SO₄, and the solvent is removed in vacuo. The product is purified by preparative HPLC (YMC-Pack Pro C18 column; 10-100% CH₃CN+0.1% CF₃COOH/H₂O+0.1% CF₃COOH within 20 min, flow 20 ml/min). The combined pure fractions are basified with solid K₂CO₃, concentrated in vacuo and the remaining aqueous phase extracted twice with CH₂Cl₂. The combined organic extracts are dried over Na₂SO₄ and evaporated in vacuo to afford the desired product as a beige solid, [M+H]⁺=583.7; t_{R} (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 3.51 min.

### EXAMPLE 394

### [4-(7-amino-3-{4-[4-(1-methyl-piperidin-4-yl)-piperazin-1-yl]-phenyl}-pyrazolo[1,5-a]pyrimidin-6-yl)-phenyl]-carbamic acid ethyl ester

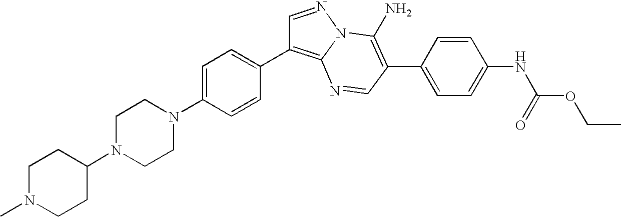

The compound is prepared in analogy to the procedure described above for example 393F) using ethyl chloroformate instead of isobutyl chloroformate. Beige solid. [M+H]⁺=555.3; t_{R} (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 2.87 min.

### EXAMPLE 395

### [4-(7-amino-3-{4-[4-(2-methoxy-ethyl)-piperazin-1-yl]-phenyl}-pyrazolo[1,5-a]pyrimidin-6-yl)-phenyl]-carbamic acid ethyl ester

### A) {4-[4-(2-Methoxy-ethyl)-piperazin-1-yl]-phenyl}-acetonitrile

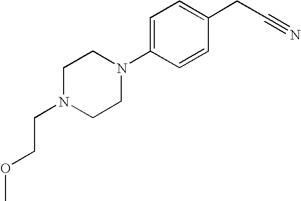

The compound is prepared in analogy to the procedure described in example 393A) using 1-(2-methoxy-ethyl)-piperazine instead of 1-(1-methyl-piperidin-4-yl)-piperazine. The crude product is purified by flash chromatography (silica gel; CH₂Cl₂/CH₃OH) to give the desired product. [M+H]⁺=260.2; t_{R} (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 3.11 min.

### B) Sodium 2-cyano-2-{4-[4-(2-methoxy-ethyl)-piperazin-1-yl]-phenyl}-ethenolate

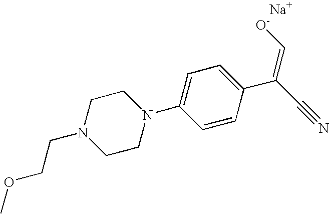

The compound is prepared in analogy to the procedure described in example 393B) using {4-[4-(2-methoxy-ethyl)-piperazin-1-yl]-phenyl}-acetonitrile instead of {4-[4-(1-methyl-piperidin-4-yl)-piperazin-1-yl]-phenyl}-acetonitrile. [M−H]-=286.2; t_{R} (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 3.07 min.

### C) 4{4-[4-(2-Methoxy-ethyl)-piperazin-1-yl]-phenyl}-2H-pyrazol-3-ylamine

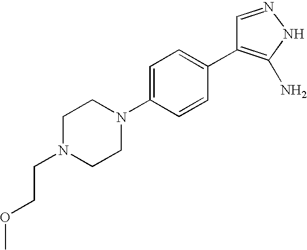

The compound is prepared in analogy to the procedure described in example 393C) using sodium 2-cyano-2-{4-[4-(2-methoxy-ethyl)-piperazin-1-yl]-phenyl}-ethenolate instead of sodium 2-cyano-2-{4-[4-(1-methyl-piperidin-4-yl)-piperazin-1-yl]-phenyl}-ethenolat. Brown solid. [M+H]⁺=302.2; t_{R} (HPLC, CC 125/4 Nucleosil 100-5 C18 AB column; 0-100% CH₃CN+0.1% CF₃COOH/H₂O+0.1% CF₃COOH for 8 min, flow 1.5 ml/min): 2.91 min.

### D) 3-{4-[4-(2-Methoxy-ethyl)-piperazin-1-yl]-phenyl}-6-(4-nitro-phenyl)-pyrazolo[1,5-a]pyrimidin-7-ylamine

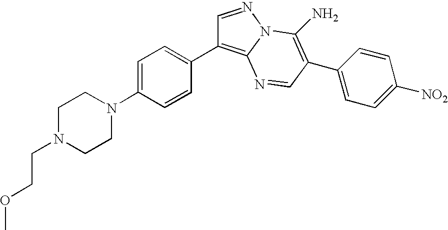

A mixture of 4-{4-[4-(2-methoxy-ethyl)-piperazin-1-yl]-phenyl}-2H-pyrazol-3-ylamine (336 mg, 1.11 mmol), 3-dimethylamino-2-(4-nitro-phenyl)-acrylonitrile (242 mg, 1.11 mmol), acetic acid (4.2 ml), ethanol (7 ml) and ˜1.25 M HCl in ethanol (3.55 ml, ˜4.44 mmol) is shaken at 85° C. for 18 h. The reaction mixture is evaporated in vacuo and the residue distributed between saturated K₂CO₃ solution and ethyl acetate. The aqueous layer is separated and extracted twice with ethyl acetate. The combined organic extracts are dried over Na₂SO₄ and evaporated in vacuo to yield the crude product as a dark solid. [M−H]-=472.3; t_{R} (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 3.01 min.

### E) 6-(4-Amino-phenyl)-3-{4-[4-(2-methoxy-ethyl)-piperazin-1-yl]-phenyl}-pyrazolo[1,5-a]pyrimidin-7-ylamine

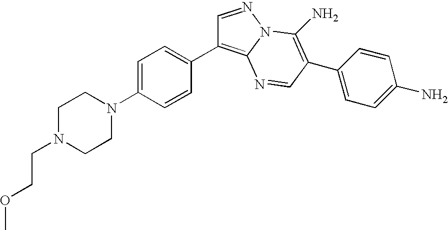

A mixture of 3-{4-[4-(2-methoxy-ethyl)-piperazin-1-yl]-phenyl}-6-(4-nitro-phenyl)-pyrazolo[1,5-a]pyrimidin-7-ylamine (340 mg, 0.72 mmol), DMF (10 ml), THF (10 ml) and Pd/C 10% (100 mg) is hydrogenated at room temperature for 14 h (hydrogen pressure ˜2 bar). The reaction mixture is filtered through a pad of celite, the residue washed with DMF and THF and the filtrate evaporated in vacuo. The crude residue is distributed between CH₂Cl₂ and half-saturated K₂CO₃ solution, the aqueous phase separated and extracted twice with CH₂Cl₂. The combined organic extracts are washed with brine, dried over Na₂SO₄ and evaporated in vacuo to yield the desired product as a dark solid. [M+H]⁺=444.3; t_{R} (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 2.41 min.

### F) [4-(7-Amino-3-{4-[4-(2-methoxy-ethyl)-piperazin-1-yl]-phenyl}-pyrazolo[1,5-a]pyrimidin-6-yl)-phenyl]-carbamic acid ethyl ester

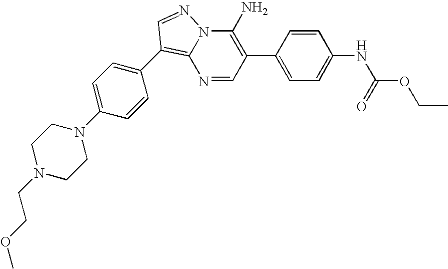

To a stirred mixture of 6-(4-amino-phenyl)-3-{4-[4-(2-methoxy-ethyl)-piperazin-1-yl]-phenyl}-pyrazolo[1,5-a]pyrimidin-7-ylamine (88.7 mg, 0.2 mmol) and pyridine (3 ml) is added ethyl chloroformate (21 μl, 0.22 mmol). After 75 min at room temperature, a second portion of ethyl chloroformate (21 μl, 0.22 mmol) is added and stirring is continued for 45 min. The reaction mixture is evaporated in vacuo and the residue distributed between 2N NaOH solution and ethyl acetate. The ethyl acetate extract is separated and the aqueous layer extracted twice with ethyl acetate. The combined organic layers are washed with brine, dried over Na₂SO₄, and the solvent is removed in vacuo. The product is purified by preparative HPLC (YMC-Pack Pro C18 column; 0-100% CH₃CN+0.1% CF₃COOH/H₂O+0.1% CF₃COOH within 20 min, flow 20 ml/min). The combined pure fractions are basified with solid K₂CO₃, concentrated in vacuo and the remaining aqueous phase extracted twice with CH₂Cl₂. The combined organic extracts are dried over Na₂SO₄ and evaporated in vacuo to afford the desired product as a beige solid. [M+H]⁺=516.3; t_{R} (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 3.17 min.

### EXAMPLE 396

### [4-(7-amino-3-{4-[4-(2-methoxy-ethyl)-piperazin-1-yl]-phenyl}-pyrazolo[1,5-a]pyrimidin-6-yl)-phenyl]-carbamic acid isobutyl ester

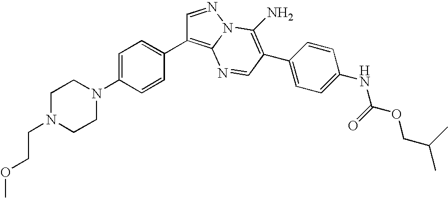

To a stirred mixture of 6-(4-amino-phenyl)-3-{4-[4-(2-methoxy-ethyl)-piperazin-1-yl]-phenyl}-pyrazolo[1,5-a]pyrimidin-7-ylamine (88.7 mg, 0.2 mmol) and pyridine (3 ml) is added isobutyl chloroformate (28.4 μl, 0.22 mmol). After 75 min at room temperature, the reaction mixture is evaporated in vacuo and the residue distributed between 2N NaOH solution and ethyl acetate. The ethyl acetate extract is separated and the aqueous layer extracted twice with ethyl acetate. The combined organic layers are washed with brine, dried over Na₂SO₄, and the solvent is removed in vacuo. The product is purified by preparative HPLC (YMC-Pack Pro C18 column; 10-100% CH₃CN+0.1% CF₃COOH/H₂O+0.1% CF₃COOH within 30 min, flow 20 ml/min). The combined pure fractions are basified with solid K₂CO₃, concentrated in vacuo and the remaining aqueous phase extracted twice with CH₂Cl₂. The combined organic extracts are dried over Na₂SO₄ and evaporated in vacuo to afford the desired product as a brownish solid. [M+H]⁺=544.8; t_{R} (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 3.81 min.

### EXAMPLE 397

### 4-{7-amino-3-[4-(4-methyl-piperazin-1-ylmethyl)-phenyl]-pyrazolo[1,5-a]pyrimidin-6-yl}-phenol

### A) [4-(4-Methyl-piperazin-1-ylmethyl)-phenyl]-acetonitrile

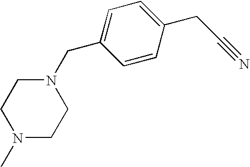

A suspension of N-methyl-piperazine (6.6 ml, 59.4 mmol), K₂CO₃ (14.87 g, 107.6 mmol) in dimethylacetamide (100 ml) is stirred at room temperature for 10 min. After addition of (4-bromomethyl-phenyl)-acetonitrile (11.3 g, 53.8 mmol) stirring is continued for 12 h. The mixture is evaporated in vacuo and the residue distributed between H₂O and ethyl acetate. The organic layer is dried over Na₂SO₄ and the solvent removed in vacuo to yield the product as an orange oil. [M+H]⁺=230.1; t_{R} (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 1.73 min.

### B) 2-[4-(4-Methyl-piperazin-1-ylmethyl)-phenyl]-3-oxo-propionitrile

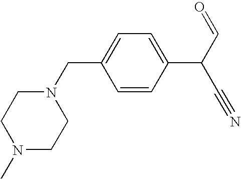

The compound is prepared in analogy to the procedure described in example 170B) using [4-(4-methyl-piperazin-1-ylmethyl)-phenyl]-acetonitrile instead of {4-[4-(1-methyl-piperidin-4-yl)-piperazin-1-yl]-phenyl}-acetonitrile. After completion of the reaction, the mixture is evaporated in vacuo. The residue is treated with H₂O, the pH adjusted to −4 by addition of acetic acid. The aqueous layer is washed with CH₂Cl₂ and evaporated in vacuo to afford the product as a yellow solid. [M+H]⁺=258.1; t_{R} (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 2.38 min.

### C) 4-[4-(4-Methyl-piperazin-1-ylmethyl)-phenyl]-2H-pyrazol-3-ylamine

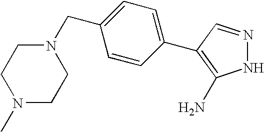

A mixture of 2-[4-(4-methyl-piperazin-1-ylmethyl)-phenyl]-3-oxo-propionitrile (8.1 g, 31.4 mmol), hydrazine monohydrate (3.82 ml, 78.6 mmol) and acetic acid (76 ml) is stirred at 100° C. for 3.5 h. After cooling to room temperature, water (165 ml) and fuming HCl (16.5 ml) are added and the mixture is stirred at 110° C. for 0.5 h. The reaction mixture is cooled down to room temperature and basified by the addition of conc. ammonia. The aqueous layer is extracted three times with CH₂Cl₂. The combined organic layers are dried over Na₂SO₄ and the solvent is removed in vacuo to give the product as an orange oil that crystallizes at room temperature. [M+H]⁺=272.1.

### D) 3-Hydroxy-2-(4-hydroxy-phenyl)-acrylonitrile

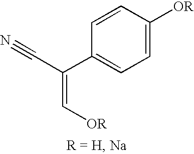

Sodium (690 mg, 30.0 mmol) is dissolved in ethanol (17 ml) at 50° C. After cooling down to room temperature, (4-hydroxy-phenyl)-acetonitrile (2.66 g, 20 mmol) and ethyl formate (2.41 ml, 30 mmol) are added and the reaction mixture is stirred at 70° C. for 2 h. After cooling down to room temperature, the precipitate is filtered off. The filtrate is evaporated to afford the green sodium salt of the product. (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 3.64 min. The precipitate of the filtration is dissolved in H₂O, the pH adjusted to ˜4 by the addition of acetic acid and the aqueous phase extracted twice with ethyl acetate. The combined organic extracts are dried over Na₂SO₄ and evaporated in vacuo to yield the product as a brown oil. [M−H]⁻=160.0

### E) 4-{7-Amino-3-[4-(4-methyl-piperazin-1-ylmethyl)-phenyl]-pyrazolo[1,5-a]pyrimidin-4-yl}-phenol

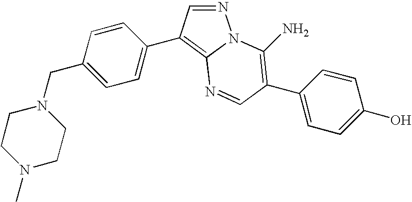

A mixture of 4-[4-(4-methyl-piperazin-1-ylmethyl)-phenyl]-2H-pyrazol-3-ylamine (120 mg, 0.44 mmol), 3-hydroxy-2-(4-hydroxy-phenyl)-acrylonitrile sodium salt (90 mg, 0.44 mmol), acetic acid (2 ml), ethanol (4 ml) and ˜1.25 M HCl in ethanol (1.76 ml, ˜2.2 mmol) is stirred at reflux for 16 h. After cooling down to room temperature, the precipitate is filtered off, washed with ethanol and dried in vacuo to afford the HCl salt of the product. [M+H]⁺=415.2; t_{R} (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 2.99 min. The filtrate is evaporated and the residue distributed between saturated K₂CO₃ solution and CH₂Cl₂. The organic layer is dried over Na₂SO₄, evaporated and the residue purified via preparative HPLC (YMC-Pack Pro C18 column; 10-100% CH₃CN+0.1% CF₃COOH/H₂O+0.1% CF₃COOH within 20 min, flow 20 ml/min). The combined pure fractions are basified with solid K₂CO₃, concentrated in vacuo and the remaining aqueous phase extracted twice with CH₂Cl₂. The combined organic extracts are dried over Na₂SO₄ and evaporated in vacuo to afford the desired product as a beige solid. [M+H]⁺=415.2; t_{R} (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 2.92 min.

### EXAMPLE 398

### [4-(7-amino-3-{4-[4-(2-hydroxy-ethyl)-piperazin-1-yl]-phenyl}-pyrazolo[1,5-a]pyrimidin-6-yl)-phenyl]-carbamic acid ethyl ester

### A) 2-(4-{4-[7-Amino-6-(4-nitro-phenyl)-pyrazolo[1,5-a]pyrimidin-3-yl]-phenyl}-piperazin-1-yl)-ethanol, hydrochloride

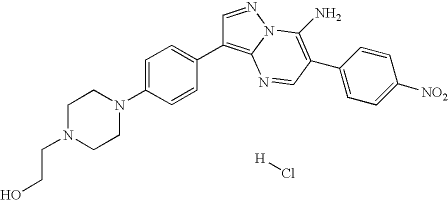

The compound is prepared in analogy to the procedure described in example 393D) using 2-{4-[4-(5-amino-1H-pyrazol-4-yl)-phenyl]-piperazin-1-yl}-ethanol instead of 4-{4-[4-(1-methyl-piperidin-4-yl)-piperazin-1-yl]-phenyl}-2H-pyrazol-3-ylamine. Greenish solid. [M+H]⁺=460.3; t_{R}(HPLC, CC 125/4 Nucleosil 100-5 C18 AB column; 5-100% CH₃CN+0.1% CF₃COOH/H₂O+0.1% CF₃COOH for 8 min, flow 1.5 ml/min): 3.37 min.

### B) 2-(4-{4-[7-Amino-6-(4-amino-phenyl)-pyrazolo[1,5-a]pyrimidin-3-yl]-phenyl}-piperazin-1-yl)-ethanol, hydrochloride

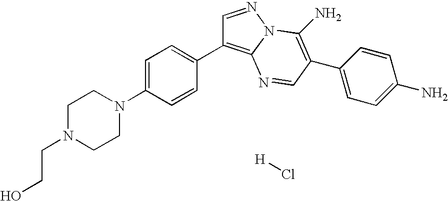

The compound is prepared in analogy to the procedure described in example 393E) using 2-(4-{4-[7-amino-6-(4-nitro-phenyl)-pyrazolo[1,5-a]pyrimidin-3-yl]-phenyl}-piperazin-1-yl)-ethanol hydrochloride instead of 3-{4-[4-(1-methyl-piperidin-4-yl)-piperazin-1-yl]-phenyl}-6-(4-nitro-phenyl)-pyrazolo[1,5-a]pyrimidin-7-ylamine hydrochloride. The crude product is treated with hot methanol, filtered, the residue washed with methanol and CH₂Cl₂ and dried in vacuo to yield the desired product as a dark beige solid. [M+H]⁺=430.2; t_{R} (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 2.46 min.

### C) [4-(7-Amino-3-{4-[4-2-hydroxy-ethyl)-piperazin-1-yl]-phenyl}-pyrazolo[1,5-a]pyrimidin-6-yl)-phenyl]-carbamic acid ethyl ester

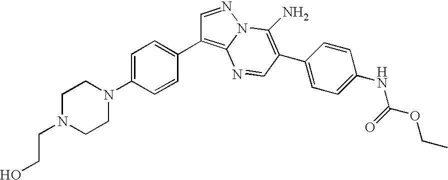

The compound is prepared in analogy to the procedure described in example 393F) but using 2(4{-[7-amino-6-(4-amino-phenyl)-pyrazolo[1,5-a]pyrimidin-3-yl]-phenyl}-piperazin-1-yl)-ethanol hydrochloride and ethyl chloroformate. The product is purified by preparative HPLC (YMC-Pack Pro C18 column; 0-100% CH₃CN+0.1% CF₃COOH/H₂O+0.1% CF₃COOH within 20 min, flow 20 ml/min). The combined pure fractions are basified with solid K₂CO₃, concentrated in vacuo and the remaining aqueous phase extracted twice with CH₂Cl₂. The combined organic extracts are dried over Na₂SO₄ and evaporated in vacuo to afford the desired product as a beige solid. [M+H]⁺=502.3; t_{R} (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 3.20 min.

### EXAMPLE 399

### (4-{7-amino-5-methyl-3-[4-(4-methyl-piperazin-1-yl)-phenyl]pyrazolo[1,5-a]pyrimidin-6-yl}-phenyl)-carbamic acid isobutyl ester

### A) 2-(4-Nitro-phenyl)-3-oxo-butyronitrile

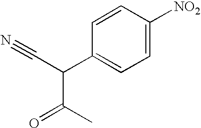

To a stirred solution of (4-nitro-phenyl)-acetonitrile (2.2 g, 13.6 mmol) in pyridine (17 ml) is added acetyl chloride (1.22 ml, 17.2 mmol) in one portion. The mixture is stirred for 20 h at room temperature and then evaporated. H₂O is added to the residue, the pH adjusted to −4 by addition of 2 N HCl and the aqueous layer extracted three times with CH₂Cl₂. The combined organic extracts are washed with H₂O, dried over Na₂SO₄ and evaporated in vacuo to yield the product as a dark brown residue. [M−H]-=203.1; t_{R} (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 4.67 min.

### B) 5-Methyl-3-[4-(4-methyl-piperazin-1-yl)-phenyl]-6-(4-nitro-phenyl)-pyrazolo[1,5-a]pyrimidin-7-ylamine, hydrochloride

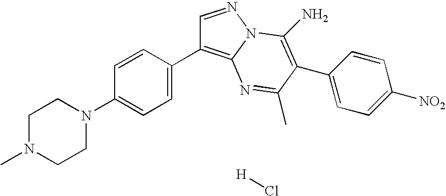

The compound is prepared in analogy to the procedure described in example 393D) but using 4-[4-(4-methyl-piperazin-1-yl)-phenyl]-2H-pyrazol-3-ylamine and 2-(4-nitro-phenyl)-3-oxo-butyronitrile. Reaction time: 120 h. Dark beige solid. [M+H]⁺=444.6; t_{R} (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 2.96 min.

### C) 6-(4-Amino-phenyl)-5-methyl-3-[4-(4-methyl-piperazin-1-yl)-phenyl]-pyrazolo[1,5-a]pyrimidin-7-ylamine, hydrochloride

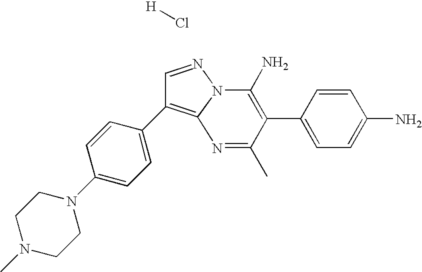

The compound is prepared in analogy to the procedure described in example 393E) but using 5-methyl-3-[4-(4-methyl-piperazin-1-yl)-phenyl]—(4-nitro-phenyl)-pyrazolo[1,5-a]pyrimidin-7-ylamine hydrochloride. The crude product is treated with methanol and CH₂Cl₂, filtered, the residue washed with methanol and CH₂Cl₂ and dried in vacuo to yield the desired product as a beige solid. [M+H]⁺=414.6; t_{R} (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 2.13 min.

### D) (4-{7-Amino-5-methyl-3-[4-(4-methyl-piperazin-1-yl)-phenyl]-pyrazolo[1,5-a]pyrimidin-6-yl}-phenyl)-carbamic acid isobutyl ester

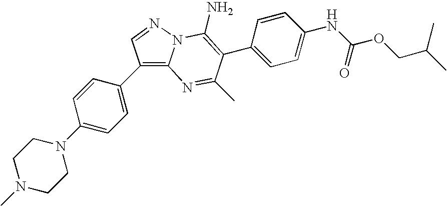

The compound is prepared in analogy to the procedure described in example 393F) but using 6-(4-amino-phenyl)-5-methyl-3-[4-(4-methyl-piperazin-1-yl)-phenyl]-pyrazolo[1,5-a]pyrimidin-7-ylamine hydrochloride. The crude product is treated with methanol, the solid filtered off, washed with methanol and ether and dried in vacuo to afford the desired product as a beige solid. [M+H]⁺=514.6; t_{R} (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 3.45 min.

### EXAMPLE 400

### (4-{7-amino-5-methyl-3-[3-(4-methyl-piperazin-1-yl)-phenyl]-pyrazolo[1,5-a]pyrimidin-6-yl}-phenyl)-carbamic acid isobutyl ester, trifluoro acetic acid salt

### A) 5-Methyl-3-[3-(4-methyl-piperazin-1-yl)-phenyl]-6-(4-nitro-phenyl)-pyrazolo[1,5-a]pyrimidin-7-ylamine, hydrochloride

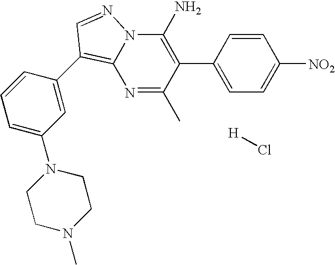

The compound is prepared in analogy to the procedure described in example 393D) but using 4-[3-(4-methyl-piperazin-1-yl)-phenyl]-2H-pyrazol-3-ylamine and 2-(4-nitro-phenyl)-3-oxo-butyronitrile. Reaction time: 140 h. Dark beige solid [M+H]⁺=444.6; t_{R} (HPLC, CC 125/4 Nucleosil 100-5 C18 AB column; 5-100% CH₃CN+0.1% CF₃COOH/H₂O+0.1% CF₃COOH for 8 min, flow 1.5 mL/min): 3.11 min.

### B) 6-(4-Amino-phenyl)-5-methyl-3-[3-(4-methyl-piperazin-1-yl)-phenyl]-pyrazolo[1,5-a]pyrimidin-7-ylamine, hydrochloride

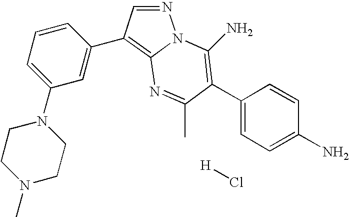

The compound is prepared in analogy to the procedure described in example 393E) but using 5-methyl-3-[3-(4-methyl-piperazin-1-yl)-phenyl]-6-(4-nitro-phenyl)-pyrazolo[1,5-a]pyrimidin-7-ylamine hydrochloride. The crude product is treated with methanol and CH₂Cl₂, filtered, the residue washed with methanol and CH₂Cl₂ and dried in vacuo to yield the desired product as a beige solid. [M+H]⁺=414.6; t_{R} (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 2.21 min.

### C) (4-{7-Amino-5-methyl-3-[3-(4-methyl-piperazin-1-yl)-phenyl]-pyrazolo[1,5-a]pyrimidin-6-yl}-phenyl)-carbamic acid isobutyl ester, trifluoro acetic acid salt

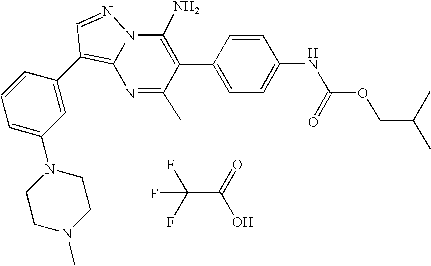

The compound is prepared in analogy to the procedure described in example 393F) but using 6-(4-amino-phenyl)-5-methyl-3-[3-(4-methyl-piperazin-1-yl)-phenyl]-pyrazolo[1,5-a]pyrimidin-7-ylamine hydrochloride. Slightly beige solid after evaporation of the pure fractions after HPLC purification. [M+H]⁺=514.7; t_{R} (HPLC, CC 125/4 Nucleosil 100-SC 18 AB column; 5-100% CH₃CN+0.1% CF₃COOH/H₂O+0.1% CF₃COOH for 8 min, flow 1.5 ml/min): 3.56 min.

### EXAMPLE 401

### 4-{7-Amino-5-methoxymethyl-3-[4-(4-methyl-piperazin-1-yl)-phenyl]-pyra zolo[1,5-a]pyrimidin-6-yl}-phenol

### A) 2-(4-Benzyloxy-phenyl)-4-methoxy-3-oxo-butyronitrile

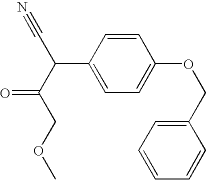

Sodium (517 mg, 22.5 mmol) is dissolved in ethanol (12.5 ml) at 50° C. After cooling to room temperature (4-benzyloxy-phenyl)-acetonitrile (3.34 g, 15 mmol) is added followed by methoxy-acetic acid methyl ester (1.49 ml, 15 mmol). The mixture is shaken during 20 h at 80° C. in a closed vial. After cooling down, the pH is adjusted to −4 by addition of 2 N HCl. The mixture is evaporated, the residue treated with H₂O and the aqueous layer extracted twice with ethyl acetate. The combined organic extracts are dried over Na₂SO₄ and evaporated in vacuo to yield the product as a dark beige solid. t_{R} (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 6.05 min.

### B) 6-(4-Benzyloxy-phenyl)-5-methoxymethyl-3-[4-(4-methyl-piperazin-1-yl)-phenyl]-pyrazolo[1,5-a]pyrimidin-7-ylamine

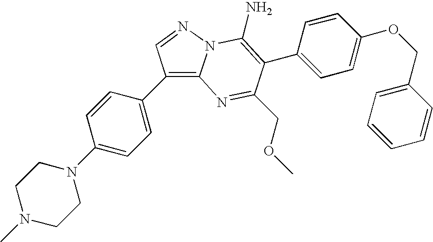

A mixture of 4-[4-(4-methyl-piperazin-1-yl)-phenyl]-2H-pyrazol-3-ylamine (1.69 g, 6.57 mmol), 2-(4-benzyloxy-phenyl)-4-methoxy-3-oxo-butyronitrile (1.94 g, 6.57 mmol), acetic acid (18 ml), ethanol (36 ml) and ˜1.25 M HCl in ethanol (21 ml, ˜26.3 mmol) is shaken for 20 h at 80° C. The mixture is evaporated in vacuo, the residue distributed between saturated K₂CO₃ solution and ethyl acetate. The aqueous layer is separated and extracted twice with ethyl acetate. The combined extracts are washed with brine, dried over Na₂SO₄ and evaporated. Methanol is added to the residue and the solid thus formed is filtered off and dried in vacuo to afford the product as a dark-brown solid. [M+H]⁺=535.3; t_{R} (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 4.63 min.

### C) 4-{7-Amino-5-methoxymethyl-3-[4-(4-methyl-piperazin-1-yl)-phenyl]-pyrazolo[1,5-a]pyrimidin-6-yl}-phenol

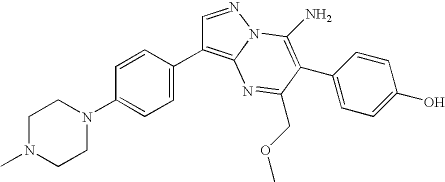

A mixture of 6-(4-benzyloxy-phenyl)-5-methoxymethyl-3-[4-(4-methyl-piperazin-1-yl)-phenyl]-pyrazolo[1,5-a]pyrimidin-7-ylamine (150 mg, 0.28 mmol), THF (3 ml), dioxane (2 ml) and Pd/C 10% (20 mg) is hydrogenated at room temperature for 16 h (hydrogen pressure ˜2 bar). The reaction mixture is filtered through a pad of celite, the residue washed with THF and the filtrate evaporated in vacuo. The crude residue is purified via flash chromatography (SiO₂, CH₂Cl₂/CH₃OH) to yield the desired product as a yellow solid. [M+H]⁺=445.3; t_{R} (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 2.97 min.

### EXAMPLE 402

### 4-(7-amino-3-{4-[4-(2-methoxy-ethyl)-piperazin-1-yl]-phenyl}-pyrazolo[1,5-a]pyrimidin-6-yl)-phenol

### A) 6-(4-Benzyloxy-phenyl)-3-{4-[4-(2-methoxy-ethyl)-piperazin-1-yl]-phenyl}-pyrazolo[1,5-a]pyrimidin-7-ylamine

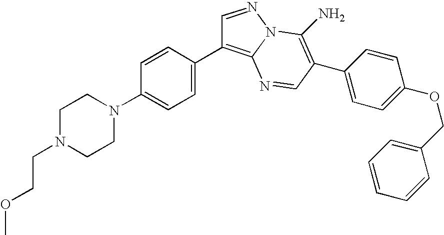

The compound is prepared in analogy to the procedure described in example 395D) but using 2-(4-benzyloxy-phenyl)-3-oxo-propionitrile. [M+H]⁺=535.3; t_{R} (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 4.38 min.

### B) 4-(7-Amino-3-{4-[4-(2-methoxy-ethyl)-piperazin-1-yl]-phenyl}-pyrazolo[1,5-a]pyrimidin-6-yl)-phenol

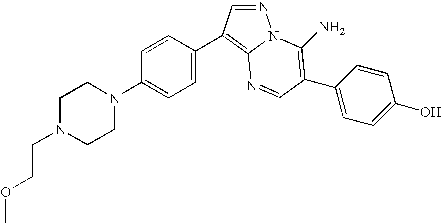

A mixture of 6-(4-benzyloxy-phenyl)-3-{4-[4-(2-methoxy-ethyl)-piperazin-1-yl]-phenyl}-pyrazolo[1,5-a]pyrimidin-7-ylamine (66.4 mg, 0.124 mmol) in CF₃COOH (3 ml) is stirred for 1 h at room temperature, evaporated in vacuo and the residue evaporated once with toluene. The product is purified by preparative HPLC (YMC-Pack Pro C18 column; 10-100% CH₃CN+0.1% CF₃COOH/H₂O+0.1% CF₃COOH within 20 min, flow 20 ml/min). The combined pure fractions are basified with solid K₂CO₃, concentrated in vacuo and the remaining aqueous phase extracted three times with CH₂Cl₂. The combined organic extracts are dried over Na₂SO₄ and evaporated in vacuo to afford the desired product as a yellow solid. [M−H]⁻=443.3; t_{R} (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 2.71 min.

### EXAMPLE 403

### 2-(4-{4-[7-amino-6-(4-benzyloxy-phenyl)-pyrazolo[1,5a]pyrimidin-3-yl]-phenyl}-piperazin-1-yl)-ethanol, hydrochloride

### A) 2-(4-Benzyloxy-phenyl)-3-oxo-propionitrile

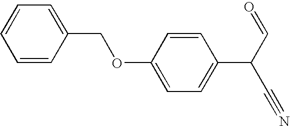

The compound is prepared in analogy to the procedure described in example 401A). Beige solid. [M+H]⁺=252.1; t_{R} (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 6.09 min

### B) 2-(4-{4-[7-amino-6-(4-benzyloxy-phenyl)-pyrazolo[1,5a]pyrimidin-3-yl]-phenyl}-piperazin-1-yl)-ethanol, hydrochloride

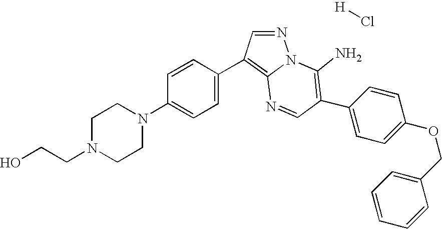

The compound is prepared in analogy to the procedure described in example 393D) but using 2-{4-[4-(5-amino-1H-pyrazol-4-yl)-phenyl]-piperazin-1-yl}-ethanol and 2-(4-benzyloxyphenyl)-3-oxo-propionitrile. [M+H]⁺=521.3; t_{R} (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 4.64 min

### EXAMPLE 404

### {7-amino-6-(4-hydroxy-phenyl)-3-[4-(4-methyl-piperazin-1-yl)-phenyl]-pyrazolo[1,5-a]pyrimidin-5-yl}-acetonitrile

### A) 4-{7-Amino-5-bromomethyl-3-[4-(4-methyl-piperazin-1-yl)-phenyl]pyrazolo[1,5-a]pyrimidin-6-yl}-phenol, hydrobromide

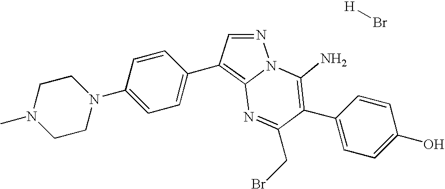

A mixture of 6-(4-benzyloxy-phenyl)-5-methoxymethyl-3-[4-(4-methyl-piperazin-1-yl)-phenyl]-pyrazolo[1,5-a]pyrimidin-7-ylamine (1.65 g, 3.1 mmol), hydrobromic acid (33%) (1.75 ml) in acetic acid (5 ml) is shaken for 16 h at 110° C. in a tightly closed flask. After cooling to 5° C. the precipitate is filtered off, washed with ether and dried in vacuo to yield the product as a beige solid. [M+H]⁺=493.1/495.1; t_{R} (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 3.65 min.

### B) {7-Amino-6-(4-hydroxy-phenyl)-3-[4-(4-methyl-piperazin-1-yl)-phenyl]-pyrazolo[1,5-a]pyrimidin-5-yl}-acetonitrile

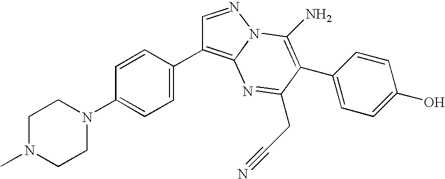

A mixture of 4-{7-amino-5-bromomethyl-3-[4-(4-methyl-piperazin-1-yl)-phenyl]pyrazolo[1,5-a]pyrimidin-6-yl}-phenol hydrobromide (1.31 g, 2.3 mmol) KCN (650 mg, 10 mmol) in DMA (10 ml) and H₂O (8 ml) is stirred for 5 h at 100° C. After evaporation in vacuo, the residue is treated with H₂O, the precipitate filtered off, washed with ethanol and ether and dried in vacuo. The crude product is purified by preparative HPLC (YMC-Pack Pro C18 column; 10-100% CH₃CN+0.1% CF₃COOH/H₂O+0.1% CF₃COOH within 20 min, flow 20 ml/min). The pure fraction is basified with 4N NaOH and extracted with ethyl acetate. The organic extract is dried over Na₂SO₄ and evaporated in vacuo to afford the desired product as a brownish solid. [M+H]⁺=440.2; t_{R} (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 3.76 min.

### EXAMPLE 405

### 4-{7-amino-5-[(2-dimethylamino-ethylamino)-methyl]-3-[4-(4-methyl-piperazin-1-yl)-phenyl]-pyrazolo[1,5-a]pyrimidin-6-yl}-phenol

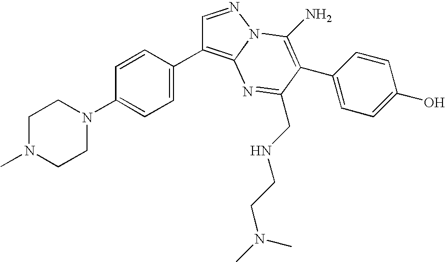

A mixture of 4-{7-amino-5-bromomethyl-3-[4-(4-methyl-piperazin-1-yl)-phenyl]pyrazolo[1,5-a]pyrimidin-6-yl}-phenol, hydrobromide (78 mg, 0.136 mmol), 2-dimethylamino-ethylamine (104 μl, 0.95 mmol), N-ethyl-diisopropylamine (62 μl, 0.36 mmol) in dimethylacetamide (1.3 ml) is heated for 15 min at 100° C. (microwave). The mixture is evaporated in vacuo and the residue purified by preparative HPLC (YMC-Pack Pro C18 column; 10-100% CH₃CN+0.1% CF₃COOH/H₂O+0.1% CF₃COOH within 20 min, flow 20 ml/min). The pure fractions are combined, basified with solid K₂CO₃, concentrated in vacuo and the aqueous layer extracted twice with CH₂Cl₂. The organic extracts are dried over Na₂SO₄ and evaporated in vacuo to afford the desired product as a brownish solid. [M+H]⁺=501.3; t_{R} (HPLC, CC 125/4 Nucleosil 100-5 C18 AB column; 5-100% CH₃CN+0.1% CF₃COOH/H₂O+0.1% CF₃COOH for 8 min, flow 1.5 ml/min): 2.91 min.

### EXAMPLE 406

### 6-(4-Amino-phenyl)-3-(4-piperazin-1-yl-phenyl)-pyrazolo[1,5-a]pyrimidin-7-ylamine

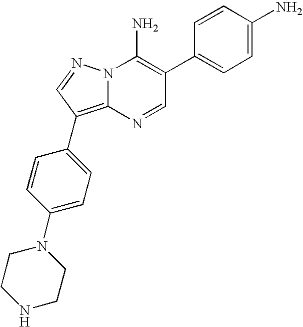

500 mg 4-{4-[7-Amino-6-(4-nitro-phenyl)-pyrazolo[1,5-a]pyrimidin-3-yl]-phenyl}-piperazine-1-carboxylic acid benzyl ester are hydrogenated under normal pressure at room temperature in N-methylpyrrolidone in the presence of 400 mg Palladium on charcoal. The mixture is filtered and evaporated in vacuo. The product is received by flash chromatography of the crude mixture (40 g silicagel 60, solvent system dichloromethane/methanol gradient. Slightly yellow solid, (M+H)⁺=386.4.

The starting material 4-{4-[7-Amino-6-(4-nitro-phenyl)-pyrazolo[1,5-a]pyrimidin-3-yl]-phenyl}-piperazine-1-carboxylic acid benzyl ester can be prepared as follows:

65 g 4-[4-(5-Amino-1H-pyrazol-4-yl)-phenyl]-piperazine-1-carboxylic acid benzyl ester and 42.5 g (Z)-3-Dimethylamino-2-(4-nitro-phenyl)-acrylonitrile are taken up in 344 ml Ethanol and 298 ml acetic acid. The mixture is heated to reflux for 5 hours and cooled to room temperature. The mixture is treated with 30% aqueous NaOH and saturated aqueous Na₂CO₃ in order to neutralize the media and then to achieve a slightly basic pH. The mixture is filtered, washed with water, ether, ethylacetate hereby removing a blue impurity. 100 mg of the crude product are separated by chromatography (12 g Redisept column, gradient methylenechloride-ethylacetate). In addition to the desired product the corresponding ethylcarbamate is formed (which can be separated by chromatography or cleaved by acidic hydrolysis in the next step). Desired product: (M+H)⁺=549.2. Ethylcarbamate: (M+H)⁺=488.

### EXAMPLE 407

### [4-(7-Amino-3-{4-[4-((S)-2-amino-3-methyl-butyryl)-piperazin-1-yl]-phenyl}-pyrazolo[1,5-a]pyrimidin-6-yl)-phenyl]-carbamic acid isobutyl ester

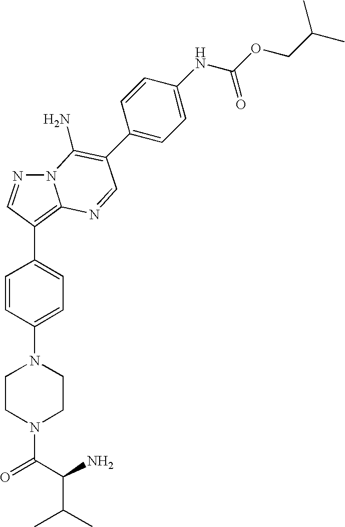

44 mg [4-(7-Amino-3-{4-[4-((S)-2-benzyloxycarbonylamino-3-methyl-butyryl)-piperazin-1-yl]-phenyl}-pyrazolo[1,5-a]pyrimidin-6-yl)-phenyl]-carbamic acid isobutyl ester in tetrahydrofurane are hydrogenated under normal pressure at room temperature in the presence of 6 mg 10% palladium on charcoal. The mixture is filtered and evacuated in vacuo. The residue is freeze-dried from tert. butanol. (M+H)⁺=585.5

The starting material can be prepared as follows:

### a) 4-(4-Cyanomethyl-phenyl)-piperazine-1-carboxylic acid benzyl ester

10.2 g 4-Bromo-phenylacetonitrile are dissolved in 107 ml dimethoxyethane and treated with 1-carbobenzyloxy-piperazine. Potassium phosphate (22.7 g), (2-Biphenyl)di-tert.-butylphosphine (4.6 g) and Palladium (II) acetate are added. The mixture is heated to reflux for 20 h under an atmosphere of Argon. After cooling the mixture is filtered and the brown filtrate is evaporated in vacuo. The crude product is separated by chromatography (400 g silicagel 60, eluent cyclohexane/ethylacetate gradient). Fractions containing the product are evaporated in vacuo to give a oil. (M+H)⁺=336.

### b) 4-[4-(1-Cyano-2-oxo-ethyl)-phenyl]-piperazine-1-carboxylic acid benzyl ester

11.7 g 4-(4-Cyanomethyl-phenyl)-piperazine-1-carboxylic acid benzyl ester are dissolved in 73 ml toluene. 4.2 ml ethylformate and 2.83 g NaOMe (powder) are added and the mixture is stirred at 38° C. for 4 h. After evaporation in vacuo the mixture is treated with methanol three times and evaporated to give a brown solid. The crude product is used in the next step without purification. (M+H)+=364.

### c) 4-[4-(5-Amino-1H-pyrazol-4-yl)-phenyl]-piperazine-1-carboxylic acid benzyl ester

To a suspension of 14.8 g 4-[4-(1-Cyano-2-oxo-ethyl)-phenyl]-piperazine-1-carboxylic acid benzyl ester in 86 ml toluene are added 7 ml acetic acid and 4.1 ml hydrazine monohydrate. The mixture is heated to reflux for 3 hours yielding a dark brown reaction mixture. After cooling 50 ml saturated aqueous solution of sodium carbonate and 50 ml of water are added. The mixture is cooled to 5° C., filtered. The solid beige residue is washed with water and dried at 50° C. in vacuo. Additional material is received after separation of the toluene phase from the biphasic filtrate, evaporation in vacuo and separation by flash chromatography (120 g silicagel, methylene chloride/methanol gradient) yielding a yellow solid. (M+H)⁺=378.

### d) 4-{4-[7-Amino-6-(4-isobutoxycarbonylamino-phenyl)-pyrazolo[1,5-a]pyrimidin-3-yl]-phenyl}-piperazine-1-carboxylic acid benzyl ester

2.51 g 4-[4-(5-Amino-1H-pyrazol-4-yl)-phenyl]-piperazine-1-carboxylic acid benzyl ester and 1.91 g [4-((Z)-1-Cyano-2-dimethylamino-vinyl)-phenyl]-carbamic acid isobutyl ester are dissolved in 19 ml acetic acid and 13.9 ml of a 1.25M solution of HCl in ethanol. The mixture is stirred at 90° C. for 4.5 hours. The mixture is poured into 180 ml of a saturated aqueous solution of sodium carbonate and extracted 3 times with methylene chloride. After drying with sodium sulfate the solution is evaporated in vacuo to give a beige solid. The crude product is purified by flash chromatography (120 g silicagel, eluent cyclohexane/ethylacetate gradient). (M+H)⁺=620.

### e) {4-[7-Amino-3-(4-piperazin-1-yl-phenyl)-pyrazolo[1,5-a]pyrimidin-6-yl]-phenyl}-carbamic acid isobutyl ester

2.7 g 4-{4-[7-Amino-6-(4-isobutoxycarbonylamino-phenyl)-pyrazolo[1,5-a]pyrimidin-3-yl]-phenyl}-piperazine-1-carboxylic acid benzyl ester are hydrogenated in methanol-tetrahydrofuran (1:1) under normal pressure at room temperature in the presence of 460 mg Palladium on charcoal. The mixture is filtered and evaporated in vacuo to give the crude product which is used in the next step without purification. (M+H)⁺=486.

### f) [4-(7-Amino-3-{4-[4-((S)-2-benzyloxycarbonylamino-3-methyl-butyryl)-piperazin-1-yl]-phenyl}-pyrazolo[1,5-a]pyrimidin-6-yl)-phenyl]carbamic acid isobutyl ester

A mixture of 41 mg {4-[7-Amino-3-(4-piperazin-1-yl-phenyl)-pyrazolo[1,5-a]pyrimidin-6-yl]-phenyl}-carbamic acid isobutyl ester, 27 mg Cbz-L-valine, 15 mg hydroxybezotriazol and 16 microlitre of triethylamine in 3 ml tetrahydrofurane are cooled to 0° C. and treated with 20 mg N-(Dimethylaminopropyl)-N ethyl-carbodiimide. After stirring at room temperature overnight the mixture is poured into 20 ml of saturated aqueous sodium carbonate and extracted with ethy acetate. The organic layer is dried, evaporated in vacuo and purified by flash chromatography (4 g silicagel, eluent methylenechloride/methanol gradient). (M+H)⁺=719.8, amorphous solid.

### Ortho-Methyl Derivatives

In analogy to example 1 the ortho-methylated compounds (R₃=Me) are made by starting from (Z)-3-Dimethylamino-2-(2-methyl-4-nitro-phenyl)-acrylonitrile which is prepared according to the following scheme:

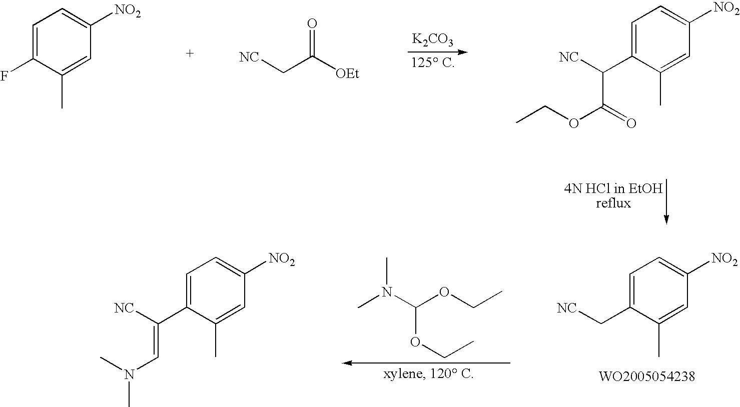

### EXAMPLES

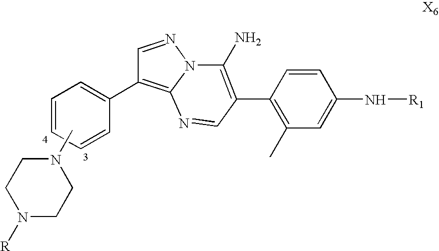

wherein R and R₁ have the significances as indicated in Table X₆ below.

TABLE X₆

| | Position on the | | | |
|---|---|---|---|---|
| Ex | phenyl | R | R₁ | M⁺H⁺ |
| 408 | 3 | Methyl | —COO-isobutyl | 514.4 |
| 409 | 3 | Methyl | —COO-tolyl | 548.4 |
| 410 | 3 | Methyl | —COO-4-Fluorophenyl | 552.3 |
| 411 | 4 | Methyl | —COO-isobutyl | 514.5 |
| 412 | 4 | Methyl | —COO-tolyl | 548.6 |
| 413 | 4 | Methyl | —COO-4-Fluorophenyl | 552.4 |

### EXAMPLE 414

### (4-{7-Amino-3-[3-(4-methyl-piperazin-1-yl)-phenyl]-pyrazolo[1,5-a]pyrimidin-6-yl}-3-methyl-phenyl)-carbamic acid isobutyl ester

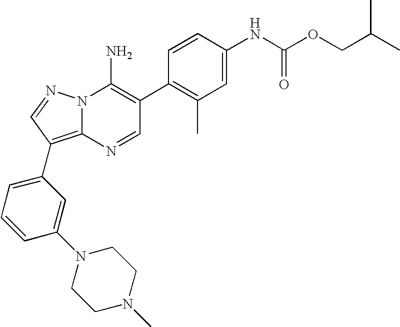

80 mg 6-(4-Amino-2-methyl-phenyl)-3-[3-(4-methyl-piperazin-1-yl)-phenyl]-pyrazolo[1,5-a]pyrimidin-7-ylamine in 5 ml N-methylpyrrolidone are treated with 50.6 microlitre chloroisobutylformiate. After stirring at room temperature for 1 hour ethylacetate is added and the mixture is extracted with aqueous sodium bicarbonate. The organic phase is dried over sodium sulfate and evaporated in vacuo to give a yellow oil. Purification by chromatography (12 g Redisep, eluent dichlormethane/methanol gradient) yields the desired product as white pulver. (M+H)+=514.4 The starting material can be prepared as follows:

### a) (Z)-3-Dimethylamino-2-(2-methyl-4-nitro-phenyl)-acrylonitrile

4.2 g (2-Methyl-4-nitro-phenyl)-acetonitrile are dissolved in 30 ml xylene and treated with 6.35 ml N,N-dimethylformamide-dimethylacetal. The mixture is heated to 120° C. for 3.5 hours, cooled, diluted with hexane and filtered. The solid material is washed with hexane and, after removing the solvent, purified by chromatography (120 g RediSep, eluent cyclohexane/dichloromethane) to give a yellow pulver. (M+H)⁺=232.2

### b) 6-(2-Methyl-4-nitro-phenyl)-3-[3-(4-methyl-piperazin-1-yl)-phenyl]-pyrazolo[1,5-a]pyrimidin-7-ylamine

1.4 g (Z)-3-Dimethylamino-2-(2-methyl-4-nitro-phenyl)-acrylonitrile and 1.56 g 4-[3-(4-Methyl-piperazin-1-yl)-phenyl]-2H-pyrazol-3-ylamine in 14 ml 1.25M HCl in ethanol and 14 ml acetic acid are heated to 130° C. overnight. After cooling methanol is added and the mixture is stirred for 20 minutes at room temperature, then filtered to give a yellow solid. The product is used in the next step without further purification. (M+H)⁺=444.6

### c) 6-(4-Amino-2-methyl-phenyl)-3-[3-(4-methyl-piperazin-1-yl)-phenyl]-pyrazolo[1,5-a]pyrimidin-7-ylamine

1.87 g 6-(2-Methyl-4-nitro-phenyl)-3-[3-(4-methyl-piperazin-1-yl)-phenyl]-pyrazolo[1,5-a]pyrimidin-7-ylamine in 200 ml methanol/tetrahydrofuran (1:1) are hydrogenated under normal pressure at room temperature in the presence of 400 mg 10% palladium on charcoal. The mixture is filtered, washed with methanol and dried in vacuo. The yellow pulver is used in the next step without purification. (M+H)+=414.6

The examples with the N-Methyl-piperazine moiety in the 4-position are prepared in an analogous way by using 4-[4-(4-Methyl-piperazin-1-yl)-phenyl]-2H-pyrazol-3-ylamine, hereby providing the respective nitro and amino intermediates:

6-(4-Amino-2-methyl-phenyl)-3-[4-(4-methyl-piperazin-1-yl)-phenyl]-pyrazolo[1,5-a]pyrimidin-7-ylamine, (M+H)+=414.5
which is received from
6-(2-Methyl-4-nitro-phenyl)-3-[4-(4-methyl-piperazin-1-yl)-phenyl]-pyrazolo[1,5-a]pyrimidin-7-ylamine, (M+H)+=444.1
Examples with two Methyl-piperazine groups:

TABLE X₇

| | |
|---|---|
| | X₇ |
| 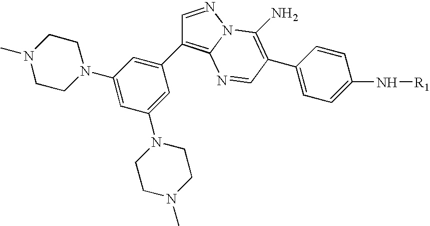 | |

| Ex | R₁ | (M + H)⁺ |
|---|---|---|
| 415 | H | 498.5 |
| 416 | 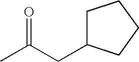 | 608.5 |
| 417 | 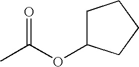 | 610.7 |

Examples 416 and 417 are prepared by acylation of example 415 in analogy to example 1. Example 415 can be prepared as follows:

### 6-(4-Amino-phenyl)-3-[3,5-bis-(4-methyl-piperazin-1-yl)-phenyl]-pyrazolo[1,5-a]pyrimidin-7-ylamine

315 mg 3-[3,5-Bis-(4-methyl-piperazin-1-yl)-phenyl]—(4-nitro-phenyl)-pyrazolo[1,5-a]pyrimidin-7-ylamine are dissolved in 50 ml methanol/dimethylformamide (1:1). After addition of 600 mg Pd on charcoal the mixture is hydrogenated at room temperature under normal pressure overnight and then the catalyst is removed by filtration. The solvent is removed in vacuo yielding the product as brown solid. (M+H)⁺=498.5

The starting material 3-[3,5-Bis-(4-methyl-piperazin-1-yl)-phenyl]-6-(4-nitro-phenyl)-pyrazolo[1,5-a]pyrimidin-7-ylamine can be prepared as follows:

### a) (3,5-Dichloro-phenyl)-acetonitrile

2.0 g 1,3-Dichloro-5-chloromethyl-benzene in 51 ml dichloromethane-water (2:1) are treated with 3.4 g tetrabutylammonium cyanide and 1.9 g sodium iodide. The mixture is stirred at room temperature overnight, the two layers are separated, the organic layer is washed with dichloromethane, dried and evaporated in vacuo. The crude product is purified by flash chromatography (80 g silicagel redisept column, cyclohexane-ethyl acetate gradient) yielding a yellow oil. ¹H-NMR (DMSO-d6): 4.1 ppm (s, benzylic protons) and others.

### b) [3,5-Bis-(4-methyl-piperazin-1-yl)-phenyl]-acetonitrile

1.0 g (3,5-Dichloro-phenyl)-acetonitrile in 27 ml dimethoxyethane are treated with 2.15 g N-methylpiperazine, 4.56 g potassium phosphate, 0.96 g (2-Biphenyl)di-tert.butylphosphine and 0.24 g palladium(II)acetate. The mixture is stirred at 84° C. for 18 hours. The cooled mixture is filtered and the dark brown filtrate is evaporated in vacuo. The crude product is purified by flash chromatography (80 g silicagel redisept column, dichloromethane (methanol gradient) yielding a brown viscous oil. (M+H)⁺=314.3

### c) 2-[3,5-Bis-(4-methyl-piperazin-1-yl)-phenyl]-3-oxo-propionitrile

1.65 g [3,5-Bis-(4-methyl-piperazin-1-yl)-phenyl]-acetonitrile in 18 ml toluene are treated with 0.64 g ethylformate and 0.43 g sodium methylate (powder). The mixture is stirred at 38° C. for 3 hours and evaporated to dryness. The product is used in the next step without purification. (M+H)⁺=342.4

### d) 4-[3,5-Bis-(4-methyl-piperazin-1-yl)-phenyl]-2H-pyrazol-3-ylamine

1.96 g 2-[3,5-Bis-(4-methyl-piperazin-1-yl)-phenyl]-3-oxo-propionitrile in 57 ml toluene are treated with 1.88 ml acetic acid and then with 1.159 hydrazine monohydrate. The mixture is heated to reflux for 3 hours yielding a yellow solution. After cooling the brown residue is treated with 100 ml 1M sodium hydroxide solution and 100 ml dichloromethane. The aqueous phase is separated, re-extracted with dichloromethane, the combined organic extracts are dried and evaporated in vacuo. Purification of the crude mixture is done by flash chromatography (120 g silicagel redisept column, dichlormethane/methanol gradient containing 1% conc. aq. ammonia. The product is received as beige amorphous solid. (M+H)⁺=356.5

### e) 3-[3,5-Bis-(4-methyl-piperazin-1-yl)-phenyl]-6-(4-nitro-phenyl)-pyrazolo[1,5-a]pyrimidin-7-ylamine

0.5 g 4-[3,5-Bis-(4-methyl-piperazin-1-yl)-phenyl]-2H-pyrazol-3-ylamine, 0.34 g (Z)-3-Dimethylamino-2-(4-nitro-phenyl)-acrylonitrile in 2.81 ml 1.25M hydrochloric acid in ethanol and 2.5 ml acetic acid are heated to reflux for 20 hours. After cooling the mixture is treated with excess 1M sodium hydroxide solution, extracted to dichloromethane/methanol (9:1), the organic layer is dried and evaporated in vacuo. The crude product is purified by flash chromatography (30 g silicagel, dichlormethane/methanol gradient containing 1% conc. aq. ammonia). (M+H)⁺=528.5

### EXAMPLE 418

### {4-[7-Amino-3-(3-piperazin-1-yl-phenyl)-pyrazolo[1,5-a]pyrimidin-6-yl]-phenyl}-carbamic acid butyl ester

### a) 4-(3-Cyanomethyl-phenyl)-piperazine-1-carboxylic acid benzyl ester

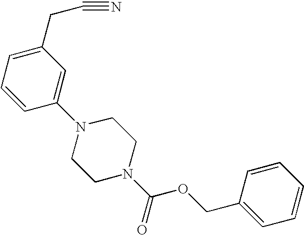

13.0 g 3-Bromo-phenylacetonitrile, 28.9 g 1-carbobenzyloxy-piperazine, 27.6 g potassium phosphate 5.8 g (2-Biphenyl)di-tert.butylphosphine and 1.5 g palladium (II) acetate are heated to reflux in 144 ml dimethoxyethane for 20 hours under an atmosphere of argon. The mixture is cooled to room temperature, filtered and the dark brown filtrate is evaporated in vacuo. The crude product is purified by flash chromatography (1000 g silicagel, cyclohexane/ethylacetate). (M+H)⁺=336.4

### b) 4-[3-(1-Cyano-2-oxo-ethyl)-phenyl]-piperazine-1-carboxylic acid benzyl ester

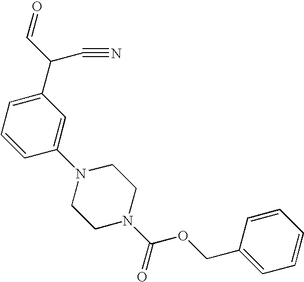

800 mg 4-(3-Cyanomethyl-phenyl)-piperazine-1-carboxylic acid benzyl ester in 8 ml toluene are treated with 288 mg ethyl formate and 193 mg sodium methylate (powder). The mixture is stirred at 38° C. for 3 hours. The thick, brown suspension is diluted with toluene in order to enable continued stirring. After an additional hour the mixture is evaporated in vacuo. The crude product is used in the next step without purification. (M+H)⁺=364

### c) 4-[3-(5-Amino-1H-pyrazol-4-yl)-phenyl]-piperazine-1-carboxylic acid benzyl ester

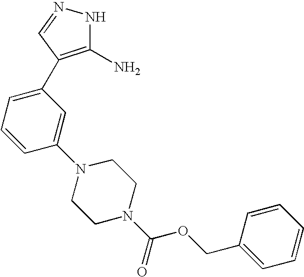

18.8 g 4-[3-(1-Cyano-2-oxo-ethyl)-phenyl]-piperazine-1-carboxylic acid benzyl ester are taken up in 83 ml toluene and 8.5 ml acetic acid. After addition of 5.18 g hydrazine monohydrate the mixture is heated to reflux for 3 hours. The yellow reaction solution is cooled, treated with saturated aq. sodium carbonate, water and ethyl acetate. The organic layer is separated and washed with aq. sodium bicarbonate, dried and evaporated in vacuo. The crude product is purified by flash chromatography (450 g silicagel, dichloromethane/methanol 95:5) yielding a yellow amorphous solid. (M+H)⁺=378.6

### d) 4-{3-[7-Amino-6-(4-nitro-phenyl)-pyrazolo[1,5-a]pyrimidin-3-yl]-phenyl}-piperazine-1-carboxylic acid benzyl ester

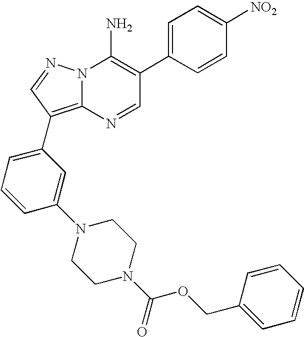

1.0 g 4-[3-(5-Amino-1H-pyrazol-4-yl)-phenyl]-piperazine-1-carboxylic acid benzyl ester are dissolved in 4.6 ml acetic acid, then treated with 576 mg (Z)-3-Dimethylamino-2-(4-nitrophenyl)-acrylonitrile and 5.3 ml of a 1.25M HCl solution in ethanol. The mixture is heated to reflux for 5.5 hours. The reaction solution is cooled to room temperature and poured into 50 ml saturated aq. sodium carbonate. After extraction with ethyl acetate the organic layer is dried, filtered (wash residue with ethyl acetate) and evaporate in vacuo. The crude product is used in the next step without purification. (M+H)⁺=551.0

### e) 4-{3-[7-Amino-6-(4-amino-phenyl)-pyrazolo[1,5-a]pyrimidin-3-yl]-phenyl}-piperazine-1-carboxylic acid benzyl ester

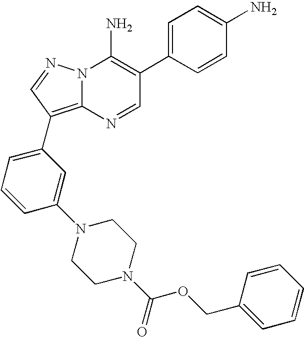

74.3 g 4-{3-[7-Amino-6-(4-nitro-phenyl)-pyrazolo[1,5-a]pyrimidin-3-yl]-phenyl}-piperazine-1-carboxylic acid benzyl ester are suspended in 800 ml tetrahydrofurane and treated with 160.2 g tin (II) chloride hydrate. The mixture is heated to reflux for 1 hour, cooled, concentrated in vacuo, diluted with ethyl acetate and treated with 4N aq. sodium hydroxide solution until a basic pH (ca. 9) is reached. The mixture is vigorously stirred and treated with ethyl acetate. The two phases are separated, the organic phase is washed with water, the combined organic phases are dried with sodium sulfate, filtered and evaporated in vacuo, hereby yielding a yellow foam. (M+H)⁺=520.4

### f) 4-{3-[7-Amino-6-(4-butoxycarbonylamino-phenyl)-pyrazolo[1,5-a]pyrimidin-3-yl]-phenyl}-piperazine-1-carboxylic acid benzyl ester

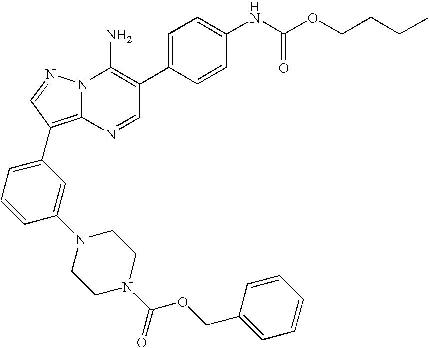

4-{3-[7-Amino-6-(4-amino-phenyl)-pyrazolo[1,5-a]pyrimidin-3-yl]-phenyl}-piperazine-1-carboxylic acid benzyl ester (1.00 g, 1.93 mM) in N-methyl-pyrrolidinone (14 ml) is cooled to 5° C. and butyl chloroformate (315 mg, 2.31 mM) is added. The reaction mixture is stirred at 5° C. for 22 h. After warming to room temperature, ethyl acetate and sat. NaHCO₃ solution are added and the layers are separated. The aqueous phase is extracted several times with ethyl acetate. The combined organic layers are dried over Na₂SO₄, and the solvent is removed in vacuo. The crude product is used in the next step without further purification. MH⁺=621.

### g) {4-[7-Amino-3-(3-piperazin-1-yl-phenyl)-pyrazolo[1,5-a]pyrimidin-6-yl]-phenyl}-carb-amic acid butyl ester

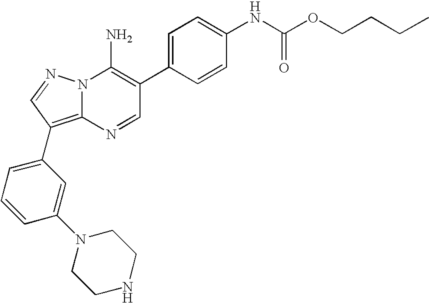

4-{3-[7-Amino-6-(4-butoxycarbonylamino-phenyl)-pyrazolo[1,5-a]pyrimidin-3-yl]-phenyl}-piperazine-1-carboxylic acid benzyl ester (905 mg, 1.46 mM) is dissolved in DMF (233 ml), palladium on carbon 10% (255 mg, 10%) is added and the reaction mixture hydrogenated at room temperature for 23 h. The reaction mixture is filtrated over celite and the solvent is removed from the filtrate in vacuo. The residue is purified by chromatography (ethyl acetate/ethanol/ammonia=90:9:1) to give the desired product as colorless crystals, MH⁺=487.

By following the procedure of above Example but using the appropriate starting materials, the compounds of formula X₉ may be prepared

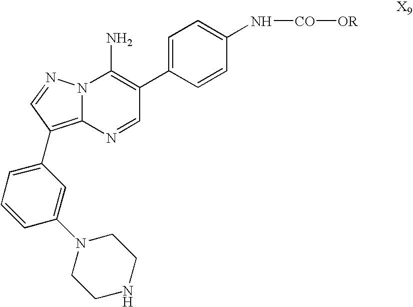

wherein R has the significance as indicated in Table X₉ below.

TABLE X₉

| Ex. | R | MH₊ |
|---|---|---|
| 419 | isobutyl | 487 |
| 420 | pentyl | 501 |
| 421 | cyclohexyl | 513 |

### EXAMPLE 422

### (4-{7-Amino-3-[3-(4-ethyl-piperazin-1-yl)-phenyl]-pyrazolo[1,5-a]pyrimidin-6-yl}-phenyl)-carbamic acid butyl ester

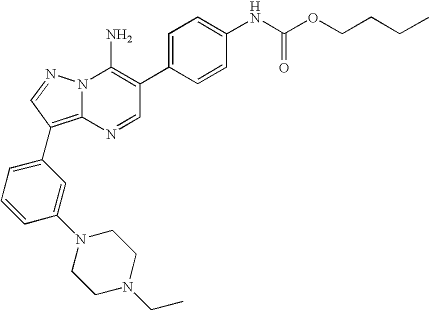

{4-[7-Amino-3-(3-piperazin-1-yl-phenyl)-pyrazolo[1,5-a]pyrimidin-6-yl]-phenyl}-carbamic acid butyl ester (100 mg, 0.21 mM) and ethyl bromide (27 mg, 0.25 mM) are dissolved in DMF (2 ml) and 3 drops of triethyl amine is added. The reaction mixture is stirred at 30° C. for 20 h. 5 Drops of water are added and the reaction mixture purified by preparative HPLC (H₂O/CH₃CN with 0.1% TFA, 9.5:0.5, 2.5 min; to H₂O/CH₃CN with 0.1% TFA, 3:7, during 45 min) to give the desired product as beige crystals, MH⁺=515.

By following the procedure of above Example but using the appropriate starting materials, the compounds of formula X₁₀ may be prepared

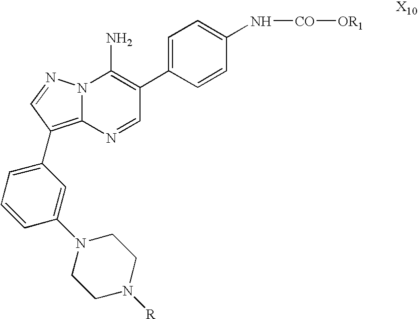

wherein R and R₁ have the significances as indicated in Table X₁₀ below.

TABLE X₁₀

| Example | R | R₁ | MH⁺ |
|---|---|---|---|
| 423 | 3-(4-ethyl-piperazin-1-yl) | pentyl | 529 |
| 424 | 3-[4-(3-piperidin-1-yl-propyl)- | pentyl | 626 |
| | piperazin-1-yl] | | |
| 425 | 3-(4-isopropyl-piperazin-1-yl) | pentyl | 543 |
| 426 | 3-[4-(2-pyrrolidin-1-yl-propyl)- | butyl | 584 |
| | piperazin-1-yl] | | |
| 427 | 3-[4-(3-piperidin-1-yl-propyl)- | butyl | 612 |
| | piperazin-1-yl] | | |
| 428 | 3-[4-(2-pyrrolidin-1-yl-propyl)- | pentyl | 594 |
| | piperazin-1-yl] | | |

### Biology/Pharmacology

The compounds of formula I and their pharmaceutically acceptable salts, exhibit valuable pharmacological properties when tested in in vitro assays, and are therefore useful as pharmaceuticals.

In particular the compounds of the invention exhibit Lck (Lymphocyte Specific Protein Tyrosi-ne Kinase) inhibiting activity, e.g. as demonstrated in accordance with the following test methods.

### 1. Biochemical Lck Kinase Assay

Enzymatic assays for the Lck, c-Src and Hck kinases of the Src family are used. The homogeneous kinase assays are based on the time-resolved fluorescence resonance energy transfer (TR-FRET) technology, more specifically it uses the LANCE technology. His-tagged wild type constructs of the kinases are used. A biotinylated, tyrosine containing peptide serves as substrate. Phosphorylation of this peptide by the kinases is quantified with an europium-labeled antiphosphotyrosine antibody (Eu-PT66) as energy donor and a streptavidin-allophycocyanine conjugate (SA-APC) as energy acceptor. The assay is established as 384-well format.

More specifically, the compounds to be tested are dissolved in pure DMSO to give a final concentration of 10 mM. For the generation of concentration-dependent response curves the compounds are diluted in 90% DMSO/10% H₂O using a PlateMate 2×2 (MATRIX) into 384-well polypropylene plates such that the highest concentration is 40 μM. These dilutions are stored at 4° C. (sealed) and may be used for up to one week. The final 1:5 dilution into dilution buffer is prepared immediately before the start of the assay. At least 8 different concentrations of test compound spanning 3 to 4 log units are used for determination of IC₅₀ values. 5 μL of these pre-dilutions are transferred into a 384-well black Optiplate used for the kinase assay which is performed in a total volume of 20 μL. This leads to a final concentration of 4.5% DMSO in the assay. The following reagents are added sequentially into each well of a 384-well black Optiplate (PerkinElmer): 5 μL compound in dilution buffer (18% DMSO) are placed into the wells using Platemate. Then 2×210 μL 2× reaction mix (as specified for Lck, c-Src and Hck, respectively) using a Multidrop384 mix on shaker. Then 5 μL enzyme in enzyme dilution buffer (80 ng/mL for either Lck, c-Src or Hck) using a Multi-channel pipette mix on shaker. Incubation is at room temperature for 120 min before the reaction is stopped by the addition of 10 μL stop buffer using a Multidrop 384 mix on shaker. The assay is developed by the addition of 45 μL detection mix using Multidrop 384 and incubated for at least 60 min at room temperature in the dark. Plate are measured using an EnVision 2102 Multilabel Reader or as backup a Wallac Victor2 1420 Multilabel Counter (excitation at 320 nm, emission at 615 nm and 665 nm). The primary data generated in a TR-FRET assay are i) fluorescence intensities at 665 nm (APC) corresponding to the FRET signal and ii) fluorescence intensities at 615 nm corresponding to the Eu³⁺ signal. If quenching of the Eu3+ fluorescence occurs then a decrease of the 615 nm (Eu³⁺) signal and of the 665 nm (APC) signal will be observed. If required, this quenching may be corrected by the following calculation of the QCV (quench corrected value): QCV=RFU(665 nm)×1000/[RFU(665 nm)+RFU(615 nm)]. Data are analyzed using Excel fit 4.0® software or Graphpad Prism 3.03®.

For all three kinases the Km values for ATP (adenosine triphosphate) have been determined: 4.6±2.2 μM for Lck, 2.3±0.9 μM for c-Src and 0.9±0.2 μM for Hck. The linearity of the reaction over the relevant time and with respect to relevant enzyme concentrations is demonstrated. The concentration of test compounds resulting in 50% inhibition of the kinase reaction (IC₅₀ value) is determined from a complete concentration-response curve with at least 8 different compound concentrations. In this assay the compounds of formula I have IC₅₀ values ranging from 0.01 nM to 1 □M. Compounds of Examples 10, 28, 65, 77, 126, 127 and 172 show IC₅₀ values of 10, 16, 25, 25, 15, 18 and 34 nM, respectively in the Lck assay.

### 2. Cellular Lck Assay

The effect of compounds to be tested on Lck-dependent phosphorylation of the T-cell signaling protein ZAP70 is assessed in Jurkat E6-1 T-cells. H₂O₂ is used to stimulate phosphorylation of signaling proteins in Jurkat T-cells. To determine the degree of Lck dependency of H₂O₂ stimulation, the effect of H₂O₂ on ZAP70 and LAT phosphorylation is evaluated in the Jurkat E6-1 and the mutant J.CAM1.6 which does not express functional Lck kinase. J.CAM1.6 cells display no detectable phosphorylation of ZAP70 Y493 nor the ZAP70 substrate LAT upon activation with 0.035% H₂O₂ as assessed by Western blotting. Stimulation of Jurkat E6-1 T-cells with 0.035% H₂O₂ results in significant intracellular phosphorylation of ZAP70 Y493 which is quantitated by flow cytometry using anti-ZAP70 pY493 antibody.

More specifically, Jurkat E6-1 are grown in RPMI 1640 containing 10% FBS and 10 ml/l of NAA-, Pen/Strep and Hepes-solutions. When a cell number of ca 1×10⁶ cells/ml is reached (cell count determined by CASI), 200 ml of cells are sedimented by centrifugation (1300 rpm, 5 min) and resuspended in 200 ml RPMI 1640 containing 0.2% FBS and 0.035% Hepes (37° C.) and incubated over night (16-19 hrs). Cells are centrifuged (1300 rpm, 5 min) and the pellet is resuspended in RPMI 1640/0.2% FBS (RT) to adjust to 4×10⁶ cells/ml (CASI count). 100 [symbol] per well of this cell suspension is added to a 96-deep well PP plate. Compounds are dissolved in DMSO or received at 10 mM DMSO solution. Serial pre-dilutions in DMSO (1:4) are made in a polypropylene microtiter plate. 5 [symbol] of the compound DMSO solution or DMSO as solvent control are added to 1000 [symbol] RPMI 1640 containing 10% FBS and 10 mM Hepes. 10% FBS is chosen to enforce potential protein binding of experimental compounds. An aliquot of 25 [symbol] of the compound/RPMI 1640 solution is added to each cell containing well. Cells are incubated with compounds at 37° C. for 1 h in a humidified incubator. Seven different concentrations are used to determine IC₅₀ values. H₂O₂ (210 [symbol] ) from a 30% stock solution is added to 30 ml RPMI 1640 containing 0.2% FBS and 10 mM Hepes. This activation solution is made briefly before the activation of the cells. 25 [symbol] of this solution is added (final concentration 0.035% (11.4 mM) per well to activate Jurkat cells. Plates are immediately vortexed and incubated in a water bath at 37° C. for 5 min. Warm 10% w/v para-formaldehyde (PF, 37° C., 37 [symbol] /well) is added to stop cellular activation (2% final concentration of PF). Cells are fixed at 37° C. for 10 min and centrifuged (1800 rpm, 5 min). Supernatant is removed by aspiration. Plates are cooled on ice for 1-2 min after which cells are permeabilized using 1 ml/well ice-cold 90% methanol (diluted with H₂O dest.). Samples are stored at −20° C. for 16 hrs On the following day 500 [symbol] PBS/2% FBS is added per well. The plate is then centrifuged (1800 rpm, 5 min). Samples are washed 2× with 1.5 ml PBS/1% FBS to re-hydrate cells. Permeabilized cells are then stained with 0.2 [symbol] rabbit anti-phospho ZAP70 Y493 specific antibody in 50 [symbol] PBS/2% FBS for 40 min at RT followed by one washing step with 1500 [symbol] PBS/1% FBS (1900 rpm, 5 min). Bound anti-ZAP70 pY493 antibody is detected using 1 [symbol] per sample of the secondary anti □rabbit IgG FITC (BD) antibody in 50 [symbol] PBS/2% FBS. Plates are incubated for 30-35 min at RT followed by a washing step with 1.6 ml PBS 2% FBS (1800 rpm, 5 min). Cell pellets are resuspended in 150 [symbol] PBS/1% FBS and transferred to a 350 [symbol] 196 well plate for flow cytometric analysis. Samples are analyzed using a FACS Calibur equipped with an auto-sampler (HTS) device. In general 10000 gated Jurkat cells are measured per sample. Light scatter signals (FSC/SSC) as well as the FITC fluorescence are acquired.

The concentration of test compounds resulting in 50% inhibition of the intracellular Lck kinase reaction (IC₅₀ value) is determined from a complete concentration-response curve with at least 7 different compound concentrations covering 3 to 4 log units. In this assay the compounds of the invention have IC₅₀ values ranging from 0.1 nM to 1 □M. Compounds of Examples 11, 19 and 173 show IC₅₀ values of 8, 59 and 27 nM, respectively.

### 2. Allogeneic Mixed Lymphocyte Reaction (MLR)

Compounds of the invention exhibit T cell inhibiting activity. More particular the compounds of the invention prevent T cell activation and/or proliferation in e.g. aqueous solution, e.g. as demonstrated in accordance with the following test method. The two-way MLR is performed according to standard procedures (J. Immunol. Methods, 1973, 2, 279 and Meo T. et al., Immunological Methods, New York, Academic Press, 1979, 227-39). Briefly, spleen cells from CBA and BALB/c mice (1.6×10⁵ cells from each strain per well in flat bottom tissue culture microtiter plates, 3.2×10⁵ in total) are incubated in RPMI medium containing 10% FCS, 100 U/ml penicillin, 100 μg/ml streptomycin (Gibco BRL, Basel, Switzerland), 50 μM 2-mercapto-ethanol (Fluka, Buchs, Switzerland) and serially diluted compounds. Seven three-fold dilution steps in duplicates per test compound are performed. After four days of incubation 1 □Ci ³H-thymidine is added. Cells are harvested after an additional five-hour incubation period, and incorporated ³H-thymidine is determined according to standard procedures. Background values (low control) of the MLR are the proliferation of BALB/c cells alone. Low controls are subtracted from all values. High controls without any sample are taken as 100% proliferation. Percent inhibition by the samples is calculated, and the concentrations required for 50% inhibition (IC₅₀ values) are determined. In this assay, the compounds of the invention have IC₅₀ values in the range of 0.01 nM to 1 □M. Compound of Examples 30 and 44 show an IC₅₀ value of 0.3 and 0.19 mM, respectively.

### 3. In Vivo Model: Mouse SEB/IL-2

The compound to be tested is administered to BALB/c mice followed e.g. 1 h later, by an intravenous administration of 3 □g per mouse of SEB to induce a rise in blood IL-2 levels. Two hours after the administration of SEB, mice are bled, and levels of IL-2 are measured in the serum using standard methods. Under control conditions (vehicle only) IL-2 concentrations measured are mostly in the range of 2000 to 8000 pg/ml. In this assay, the compounds of formula I inhibit IL-2 secretion when administered orally e.g. at a dose of from 50 to 120 mg/kg; for example, Compound of Example 10 inhibits the secretion of IL-2 by 59% at e.g. 100 mg/kg po.

The compounds of formula I are therefore useful in the prevention or treatment of disorders or diseases where Lck plays a role, e.g. diseases or disorders mediated by immune cells including e.g. T lymphocytes, NK cells, B lymphocytes, e.g. acute or chronic rejection of organ or tissue allo- or xenografts, atherosclerosis, vascular occlusion due to vascular injury such as angioplasty, restenosis, fibrosis (especially pulmonary, but also other types of fibrosis, such as renal fibrosis), angiogenesis, hypertension, heart failure, chronic obstructive pulmonary disease, CNS disease such as Alzheimer disease or amyotrophic lateral sclerosis, cancer, infectious disease such as AIDS, septic shock or adult respiratory distress syndrome, ischemia/reperfusion injury e.g. myocardial infarction, stroke, gut ischemia, renal failure or hemorrhage shock, or traumatic shock.

The compounds of formula I are also useful in the treatment and/or prevention of acute or chronic inflammatory diseases or disorders or auto immune diseases e.g. sarcoidosis, fibroid iung, idiopathic interstitial pneu-monia, obstructive airways disease, including conditions such as asthma, intrinsic asthma, extrinsic asthma, dust asthma, particularly chronic or inveterate asthma (for example late asthma and airway hyper-responsiveness), bronchitis, including bronchial asthma, infantile asthma, rheumatoid arthritis, osteoarthritis, systemic lupus erythematosus, nephrotic syn-drome lupus, Hashimoto's thyroiditis, multiple sclerosis, myasthenia gravis, type I diabetes mellitus and complications associated therewith, type II adult onset diabetes mellitus, uveitis, nephrotic syndrome, steroid dependent and steroid-resistant nephrosis, palmoplantar pus-tulosis, allergic encephalomyelitis, glomerulonephritis, psoriasis, psoriatic arthritis, atopic eczema (atopic dermatitis), allergic contact dermatitis, irritant contact dermatitis and further eczematous dermatitises, seborrheic dermatitis, lichen planus, pemphigus, bullous pemphigoid, epidermolysis bullosa, urticaria, angioedemas, vasculitides, erythemas, cutaneous eosi-nophilias, acne, alopecia areata, eosinophilic fasciitis, atherosclerosis, conjunctivitis, kerato-conjunctivitis, keratitis, vernal conjunctivitis, uveitis associated with Behcet's disease, herpe-tic keratitis, conical cornea, Sjoegren [symbol] syndrome, dystorphia epithelialis corneae, keratoleukoma, ocular pemphigus, Mooren's ulcer, scleritis, Graves' ophthalmopathy, severe intraocular inflammation, inflammation of mucosa or blood vessels such as leukotriene B4-mediated diseases, gastric ulcers, vascular damage caused by ischemic diseases and thrombosis, ischemic bowel disease, inflammatory bowel disease (e.g. Crohn's disease or ulcerative colitis), necrotizing enterocolitis, renal diseases including interstitial nephritis, Goodpasture's syndrome hemolytic uremic syndrome and diabetic nephropathy, nervous diseases selected from multiple myositis, Guillain-Barre syndrome, Meniere's disease and radiculopathy, collagen disease including scleroderma, Wegener's granuloma and Sjogren' syndrome, chronic autoimmune liver diseases including autoimmune hepatitis, primary biliary cirrhosis and sclerosing cholangitis), partial liver resection, acute liver necrosis (e.g. necrosis caused by toxins, viral hepatitis, shock or anoxia), cirrhosis, fulminant hepatitis, pustular psoriasis, Behcet's disease, active chronic hepatitis, Evans syndrome, pollinosis, idiopathic hypoparathyroidism, Addison disease, autoimmune atrophic gastritis, lupoid hepatitis, tubulointerstitial nephritis, membranous nephritis, or rheumatic fever. The compounds of formula I are useful for treating tumors, e.g. where Src kinases, in particular Lck, play a role in cell proliferation/differentiation such as T-lymphoblastic leukemia, mammary cancer, genitourinary cancer, lung cancer, gastrointestinal cancer, epidermoid cancer, melanoma, ovarian cancer, pancreas cancer, neuroblastoma, head and/or neck cancer or bladder cancer, or in a broader sense renal, brain or gastric cancer; in particular (i) a breast tumor; an epidermoid tumor, such as an epidermoid head and/or neck tumor or a mouth tumor; a lung tumor, for example a small cell or non-small cell lung tumor; a gastrointestinal tumor, for example, a colorectal tumor; or a genitourinary tumor, for example, a prostate tumor (especially a hormone-refractory prostate tumor); or (ii) a proliferative disease that is refractory to the treatment with other chemotherapeutics; or (iii) a tumor that is refractory to treatment with other chemotherapeutics due to multidrug resistance. They are also useful for treating tumors of blood and lymphatic system (e.g. Hodgkin [symbol] disease, Non-Hodgkin [symbol] lym-phoma, Burkitt [symbol] lymphoma, AIDS-related lymphomas, malignant immunoproliferative disea-ses, multiple myeloma and malignant plasma cell neoplasms, lymphoid leukemia, acute or chronic myeloid leukemia, acute or chronic lymphocytic leukemia, monocytic leukemia, other leukemias of specified cell type, leukemia of unspecified cell type, other and unspecified malignant neoplasms of lymphoid, haematopoietic and related tissues, for example diffuse large cell lymphoma, T-cell lymphoma or cutaneous T-cell lymphoma). Myeloid cancer includes e.g. acute or chronic myeloid leukaemia.

Where a tumor, a tumor disease, a carcinoma or a cancer are mentioned, also metastasis in the original organ or tissue and/or in any other location are implied alternatively or in addition, whatever the location of the tumor and/or metastasis.

For the above uses the required dosage will of course vary depending on the mode of administration, the particular condition to be treated and the effect desired. In general, satisfactory results are indicated to be obtained systemically at daily dosages of from about 0.2 to 2.5 mg/kg per body weight. An indicated daily dosage in the larger mammal, e.g. humans, is in the range from about 2 mg to about 2 g, conveniently administered, for example, in divided doses up to four times a day or in retard form. Suitable unit dosage forms for oral administration comprise from ca. 0.5 mg to 1 g active ingredient.

The compounds of the invention may be administered by any conventional route, in particular parenterally, for example in the form of injectable solutions or suspensions, enterally, e.g. orally, for example in the form of tablets or capsules, topically, e.g. in the form of lotions, gels, ointments or creams, or in a nasal or a suppository form. Topical administration is e.g. to the skin. A further form of topical administration is to the eye. Pharmaceutical compositions comprising a compound of the invention in association with at least one pharmaceutical acceptable carrier or diluent may be manufactured in conventional manner by mixing with a pharmaceutically acceptable carrier or diluent.

The compounds of formula I may be administered in free form or in pharmaceutically acceptable salt form, e.g. as indicated above. Such salts may be prepared in conventional manner and exhibit the same order of activity as the free compounds.

In accordance with the foregoing, the present invention also provides:

(1) A compound of formula I or a pharmaceutically acceptable salt thereof, for use as a pharmaceutical;
(2) A compound of formula I or a pharmaceutically acceptable salt thereof, for use as a Lck inhibitor, for example for use in any of the particular indications hereinbefore set forth;
(3) A pharmaceutical composition, e.g. for use in any of the indications herein before set forth, comprising a compound of formula I or a pharmaceutically acceptable salt thereof, together with one or more pharmaceutically acceptable diluents or carriers therefor.
(4) A method for the treatment of any of particular indication hereinbefore set forth in a subject in need thereof which comprises administering to the subject an effective amount of a compound of formula I or a pharmaceutically acceptable salt thereof;
(5) The use of a compound of formula I or a pharmaceutically acceptable salt thereof, for the manufacture of a medicament for the treatment or prevention of a disease or condition in which Lck activation plays a role or is implicated; e.g. as discussed above.

The compounds of formula I may be administered as the sole active ingredient or in conjunction with, e.g. as an adjuvant to, other drugs e.g. in immunosuppressive or immunomodulating regimens or other anti-inflammatory agents, e.g. for the treatment or prevention of allo- or xenograft acute or chronic rejection or inflammatory or autoimmune disorders, a chemotherapeutic agent or an anti-infective agent, e.g. an anti-viral agent such as e.g. an anti-retroviral agent or an antibiotic. For example, the compounds of formula I may be used in combination with a calcineurin inhibitor, e.g. cyclosporin A, ISA 247 or FK 506; an mTOR inhibitor, e.g. rapamycin, 40-O-(2-hydroxyethyl)-rapamycin, CC1779, ABT578, biolimus-7, biolimus-9, TAFA-93, AP23573, AP23464, or AP23841; an ascomycin having immuno-suppressive properties, e.g. ABT-281, ASM981, etc.; corticosteroids; cathepsin S inhibitors; cyclophosphamide; azathioprine; methotrexate; leflunomide; mizoribine; mycophenolic acid; mycophenolate mofetil; 15-deoxyspergualine or an immunosuppressive homologue, analogue or derivative thereof; a PKC inhibitor, e.g. as disclosed in WO O₂/38561 or WO 03/82859, e.g. the compound of Example 56 or 70; a JAK3 kinase inhibitor, e.g. N-benzyl-3,4-dihydroxy-benzylidene-cyanoacetamide α-cyano-(3,4-dihydroxy)-]N-benzylcinnamamide (Tyrphostin AG 490), prodigiosin 25-C (PNU156804), [4-(4′-hydroxyphenyl)-amino-6,7-dimethoxyquinazoline] (WHI-P131), [4-(3′-bromo-4′-hydroxy-phenyl)-amino-6,7-dimethoxyquinazoline] (WHI-P154), [4-(3′,5′-dibromo-4′-hydroxyphenyl)-amino-6,7-dimethoxyquinazoline] WHI-P97, KRX-211, 3-{(3R,4R)-4-methyl-3-[methyl-(7H-pyrrolo[2,3-d]pyrimidin-4-yl)-amino]-piperidin-1-y}-3-oxo-propionitrile, in free form or in a pharmaceutically acceptable salt form, e.g. mono-citrate (also called CP-690,550), or a compound as disclosed in WO 04/052359 or WO 05/066156; a S1P receptor agonist or modulator, e.g. FTY720 optionally phosphorylated or an analog thereof, e.g. 2-amino-2-[4-(3-benzyloxyphenylthio)-2-chlorophenyl]ethyl-1,3-propanediol optionally phosphorylated or 1-{4-[1-(4-cyclohexyl-3-trifluoromethyl-benzyloxyimino)-ethyl]-2-ethyl-benzyl}-azetidine-3-carboxylic acid or its pharmaceutically acceptable salts; monoclonal antibodies to leukocyte receptors, e.g., MHC, CD2, CD3, CD4, CD7, CD8, CD11a/CD18, CD25, CD27, CD28, CD40. CD45, CD58, CD80, CD86, CD137, ICOS, CD150 (SLAM), OX40, 4-1 BB or to their ligands, e.g. CD154, or antagonists thereof; other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4Ig (for ex. designated ATCC 68629) or a mutant thereof, e.g. LEA29Y; adhesion molecule inhibitors, e.g. LFA-1 antagonists, ICAM-1 or -3 antagonists, VCAM-4 antagonists or VLA-4 antagonists, e.g. natalizumab (ANTEGREN®); or antichemokine antibodies or antichemokine receptor antibodies or low molecular weight chemokine receptor antagonists, e.g. anti MCP-1 antibodies.

A compound of formula I may also be used in combination with other antiproliferative agents. Such antiproliferative agents include, but are not limited to:

(i) aromatase inhibitors, e.g. steroids, especially exemestane and formestane and, in particular, non-steroids, especially aminoglutethimide, vorozole, fadrozole, anastrozole and, very especially, letrozole;
(ii) antiestrogens, e.g. tamoxifen, fulvestrant, raloxifene and raloxifene hydrochloride;
(iii) topoisomerase I inhibitors, e.g. topotecan, irinotecan, 9-nitrocamptothecin and the macromolecular camptothecin conjugate PNU-166148 (compound A1 in WO99/17804);
(iv) topoisomerase II inhibitors, e.g. the antracyclines doxorubicin (including liposomal formulation, e.g. CAELYX™), epirubicin, idarubicin and nemorubicin, the anthraquinones mitoxantrone and losoxantrone, and the podophillotoxines etoposide and teniposide;
(v) microtubule active agents, e.g. the taxanes paclitaxel and docetaxel, the vinca alkaloids, e.g., vinblastine, especially vinblastine sulfate, vincristine especially vincristine sulfate, and vinorelbine, discodermolide and epothilones, such as epothilone B and D;
(vi) alkylating agents, e.g. cyclophosphamide, ifosfamide and melphalan;
(vii) histone deacetylase inhibitors;
(viii) famesyl transferase inhibitors;
(ix) COX-2 inhibitors, e.g. celecoxib (Celebrex®), rofecoxib (Vioxx®) and lumiracoxib (COX189);
(x) MMP inhibitors;
(xi) mTOR inhibitors;
(xii) antineoplastic antimetabolites, e.g. 5-fluorouracil, tegafur, capecitabine, cladribine, cytarabine, fludarabine phosphate, fluorouridine, gemcitabine, 6-mercaptopurine, hydroxyurea, methotrexate, edatrexate and salts of such compounds, and furthermore ZD 1694 (RALTITREXED™), LY231514 (ALIMTA™), LY264618 (LOMOTREXOL™) and OGT719;
(xiii) platin compounds, e.g. carboplatin, cis-platin and oxaliplatin;
(xiv) compounds decreasing the protein kinase activity and further anti-angiogenic compounds, e.g. (i) compounds which decrease the activity of the Vascular Endothelial Growth Factor (VEGF) (b) the Epidermal Growth Factor (EGF), c-Src, protein kinase C, Platelet-derived Growth Factor (PDGF), Bcr-Abl tyrosine kinase, c-kit, Flt-3 and Insulin-like Growth Factor I Receptor (IGF-IR) and Cyclin-dependent kinases (CDKs); (ii) Imatinib, midostaurin, Iressa™ (ZD1839), CGP 75166, vatalanib, ZD6474, GW2016, CHIR-200131, CEP-7055/CEP-5214, CP-547632 and KRN-633; (iii) thalidomide (THALOMID), celecoxib (Celebrex), SU5416 and ZD6126;
(xv) gonadorelin agonists, e.g. abarelix, goserelin and goserelin acetate;
(xvi) anti-androgens, e.g. bicalutamide (CASODEX™);
(xvii) bengamides;
(xviii) bisphosphonates, e.g. etridonic acid, clodronic acid, tiludronic acid, pamidronic acid, alendronic acid, ibandronic acid, risedronic acid and zoledronic acid;
(xix) antiproliferative antibodies, e.g. trastuzumab (Herceptin™), Trastuzumab-DM1, erlotinib (Tarceva™), bevacizumab (Avastin™), rituximab (Rituxan®), PRO64553 (anti-CD40) and 2C4 Antibody;
(xx) temozolomide (TEMODAL®).

The structure of the active agents identified by code nos., generic or trade names may be taken from the actual edition of the standard compendium □The Merck Index□ or from databases, e.g. Patents International (e.g. IMS World Publications).

In accordance with the foregoing the present invention provides in a yet further aspect:

(6) A method as defined above comprising co-administration, e.g. concomitantly or in sequence, of a therapeutically effective amount of a) a compound of formula I or a pharmaceutically acceptable salt thereof, and b) a second drug substance, said second drug substance being for example for use in any of the particular indications hereinbefore set forth.
(7) A combination comprising a therapeutically effective amount of a Lck inhibitor, e.g. a compound of formula I or a pharmaceutically acceptable salt thereof, and a second drug substance, said second drug substance being for example as disclosed above.

Where a Lck inhibitor, e.g. a compound of formula I, is administered in conjunction with other immunosuppressive/immunomodulatory, anti-inflammatory or antineoplastic agent, e.g. as disclosed above, dosages of the co-administered drug or agent will of course vary depending on the type of co-drug or □agent employed, or the specific drug or agent used, or the condition being treated and so forth.

1. A compound of formula I 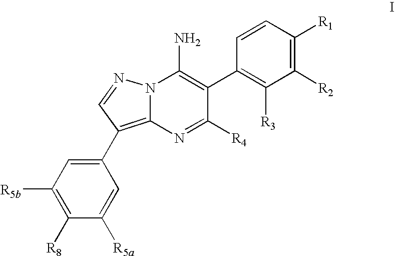
wherein
R₁ and R₂ are independently selected from the group consisting of H; OH; NH₂; NO₂; C₁₋₄alkyl; C₁₋₄alkoxy; aryl-C₁₋₄alkoxy; NR₁₁SO₂R₁₂; NR₁₃COR₁₄; NR₁₅COOR₁₆ and NR₁₇CONR₁₈R₁₉; provided that at least one of R₁ and R₂ is other than H;
R₃ is H; halogen; C₁₋₄alkyl; or C₁₋₄alkoxy;
R₄ is H; optionally substituted C₁₋₄alkyl; or C₁₋₄alkoxy optionally substituted with NH(C₁₋₄alkyl) or N(C₁₋₄alkyl)₂;
R₅ₐ, R_{5b} and R₆, are independently selected from the group consisting of H; OH; OR_{c} wherein R_{c} is C₁₋₄alkyl; and a residue of formula (a) 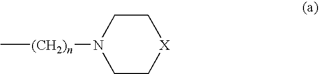
provided that at least one of R₅ₐ, R_{5b} and R₆ is other than H;
R₁₁ is H; or optionally substituted C₁₋₄alkyl;
R₁₂ is C₁₋₈alkyl; C₃₋₈cycloalkyl; optionally substituted-aryl or aryl-C₁₋₄alkyl; heterocycyl; optionally substituted heteroaryl or heteroaryl-C₁₋₄alkyl;
R₁₃ is H; or optionally substituted C₁₋₄alkyl;
R₁₄ is optionally substituted C₁₋₈alkyl; optionally substituted C₃₋₈ cycloalkyl; optionally substituted aryl or aryl-C₁₋₄alkyl; or optionally substituted heteroaryl or heteroaryl-C₁₋₄alkyl;
R₁₅ is H; or C₁₋₄alkyl;
R₁₆ is optionally substituted C₁₋₈alkyl; C₃₋₆alkenyl; C₃₋₆alkynyl; optionally substituted C₃₋₈cycloalkyl; optionally substituted aryl or aryl-C₁₋₄alkyl; or optionally substituted heteroaryl-C₁₋₄ alkyl;
R₁₇ and R₁₈, are independently selected from the group consisting of H; and C₁₋₄alkyl,
R₁₉ is C₁₋₈alkyl optionally substituted with halogen or cyano; C₃₋₈cycloalkyl; aryl or aryl-C₁₋₄ alkyl, in which each aryl ring is optionally substituted with halogen, halo-C₁₋₄alkyl, halo-C₁₋₄alkoxy or heterocyclyl; or R₁₉ is optionally substituted heteroaryl or heterocyclyl;
or R₁₈ and R₁₉ form together with the nitrogen atom to which they are bound an optionally substituted heterocyclyl;
n is 0 or 1;
X is CR₂₀R₂₁, wherein R₂₀ and R₂₁ are independently selected from the group consisting of H, C₁₋₄alkyl; O; and N—R₂₂
wherein R₂₂ is H; optionally substituted C alkyl; optionally substituted aryl-C₄alkyl; optionally substituted heteroaryl-C₁₋₄alkyl; optionally substituted heterocyclyl; SO₂—C₁₋₄alkyl; CO—R₂₃— wherein R₂₃ is C₁₋₄alkyl optionally substituted with halogen, heterocyclyl, heteroaryl, amino and/or COOH, or R₂₃ is optionally substituted aryl, heteroaryl or heterocyclyl; or R₂₂ is CO—CHR₂₄—NR₂₅R₂₆ wherein R₂₄ is H, C₁₋₈alkyl optionally substituted with OH, NH₂, NH(C₁₋₄alkyl), N(C₁₋₄alkyl)₂, COOH, carbamoyl, CONH(C₁₋₄alkyl), CON(C₁₋₄alkyl)₂ or optionally substituted aryl or heteroaryl, and wherein R₂₅ is H or C₁₋₄alkyl, and R₂₆ is H, C₁₋₄alkyl, C₁₋₄alkoxy-carbonyl or aryl-C₁₋₄alkoxycarbonyl wherein aryl may be optionally substituted,
provided that
i. when either R₅ₐ, R_{5b} or R₆ is a residue of formula (a) wherein X is NCH₃ and n is 0, then either R₂ is other than NH—SO₂—CH₃ or NH—SO₂-4-fluoro-phenyl or R₁ is other than NH—SO₂2,3-dichloro-phenyl or R₃ or R₄ is other than H;
ii. when either R₅ₐ, R_{5b} or R₆ is a residue of formula (a) wherein X is NCH₃ and n is 0, then either R₂ is other than NH—CO—CH₃ or R₃ or R₄ is other than H;
iii. when either R₅ₐ, R_{5b} or R₆ is a residue of formula (a) wherein X is NH or NCH₃ and n is 0, then either R₂, is other than NH—COOC₁₋₂alkyl or R₃ or R₄ is other than H;
iv. when either R₅ₐ, R_{5b} or R₆ is a residue of formula (a) wherein X is NH or NCH₃ and n is 0, then either R₁ is other than —NH—CO—NH-(3-CF₃-4-morpholino-phenyl) or R₂ is other than NH—CO—NH-(3CF₃-phenyl) or R₃ or R₄ is other than H;
v. when one of R₁ and R₂ is OH, the other is H, R₄ is H and only one of R₅ₐ, R_{5b} or R₆ is a residue of formula (a) and the remaining being each H, then the residue of formula (a) is other than 4-methyl-piperazinyl;
vi. when one of R₁ and R₂ is OH, the other is H and only one of R₅ₐ, R_{5b} or R₆ is a 4-methyl-piperazinyl, the remaining being each H, then R₄ is optionally substituted C₁₋₄alkyl; and
vii. when either R₅ₐ, R_{5b} or R₆ is a residue of formula (a) wherein X is NH or NCH₃ and n is 0, and R₁ is H, then R₂ is other than NH₂ or R₃ or R₄ is other than H; or a salt thereof.

2. The compound according to claim 1, wherein R₅ₐ, R_{5b} and R₆ are independently selected from the group consisting of H; OH; and a residue of formula (a), provided that at least one of R₅ₐ, R_{5b} and R₆ is other than H.

3. The compound of claim 1, wherein R₅ₐ, R_{5b} and R₆, are independently selected from the groups consisting of H; and a residue of formula (a), provided that at least one of R₅ₐ, R_{5b} and R₆ is other than H.

4. The compound of claim 1, wherein R₁ is NR₁₁SO₂R₁₂; NR₁₃COR₁₄; NR₁₅COOR₁₆; or NR₁₇CONR₁₈R₁₉.

5. The compound of claim 1, wherein R₁ is NR₁₁SO₂R₁₂; NR₁₃COR₁₄; NR₁₅COOR₁₆; or NR₁₇CONR₁₈R₁₉, and wherein R₅ₐ, R_{5b} and R₆ are independently selected from the group consisting of H; and a residue of formula (a), provided that at least one of R₅ₐ, R_{5b} and R₆ other than H.

6. The compound according to claim 1 wherein R₂ is H, OH, C₁₋₄alkyl, or C₁₋₄alkoxy.

7. (canceled)

8. (canceled)

9. A pharmaceutical composition, comprising: the compound of formula I according to claim 1 or a pharmaceutically acceptable salt thereof, and one or more pharmaceutically acceptable diluents or carriers therefor.

10. (canceled)

11. A combination comprising a therapeutically effective-amount of the compound of formula I according to claim 1 or a pharmaceutically acceptable salt thereof, and a second drug substance.

12. (canceled)

13. A method of treating a disease or condition wherein inhibition of Lck kinase in needed, comprising: administering to a patient in need thereof, an effective amount of the compound of formula (I) according to claim 1, or a pharmaceutically acceptable salt thereof.

14. The method of claim 13 in which the disease or condition is an autoimmune disease, an acute or chronic inflammatory disease, or an acute or chronic rejection of organ or tissue allo- or xenografts.
